(19) United States
(12) Patent Application Publication
(10) Pub. No.: US 2026/0306471 A1
(43) Pub. Date: Oct. 8, 2026

(54) PROLYL HYDROXYLASE DOMAIN-CONTAINING PROTEIN (PHD) INHIBITORS, COMBINATIONS AND USES THEREOF

(71) Applicant: INSILICO MEDICINE IP LIMITED, Hong Kong (CN)

(72) Inventors: Yanyun FU, Shanghai (CN); Man ZHANG, Shanghai (CN); Qingyuan MENG, Shanghai (CN); Xiao DING, Shanghai (CN); Liena QIN, Shanghai (CN); Feng REN, Shanghai (CN); Jianyu XU, Shanghai (CN)

(21) Appl. No.: 19/479,259

(22) PCT Filed: Apr. 26, 2024
(86) PCT No.: PCT/CN2024/090111

§ 371 Date: Oct. 27, 2025

(30) Foreign Application Priority Data
Apr. 28, 2023 (WO) PCT/CN2023/091788

(51) Int. Cl. A61K 31/444; A61K 31/194; A61K 31/196; A61K 31/28; A61K 31/365; A61K 31/42; A61K 31/573; A61K 31/635; A61K 38/00; A61K 45/06; A61P 1/00; C07K 16/24

(52) U.S. Cl. CPC A61K 31/444; A61K 31/194; A61K 31/196; A61K 31/28; A61K 31/365; A61K 31/42; A61K 31/573; A61K 31/635; A61K 38/00; A61K 45/06; A61P 1/00; C07K 16/241

(57) ABSTRACT

Described herein are methods of inflamatory bowel disease using a Prolyl Hydroxylase Domain-Containing Protein (PHD) inhibitor and an additional agent.

### CROSS-REFERENCE

This patent application claims the benefit of International Application No. PCT/CN2023/091788, filed Apr. 28, 2023, which is incorporated herein by reference in its entirety.

### BACKGROUND

Hypoxia-inducible factor (HIF) mediates gene expression in response to changes in cellular oxygen concentration. HIF is a heterodimer having an oxygen-regulated subunit (HIF-α) and a constitutively expressed subunit (HIF-β). HIF prolyl hydroxylase, which is also known as prolyl hydroxylase domain-containing protein (PHD), exists as three isoforms in humans (PHD1, PHD2, and PHD3). PHDs act as oxygen sensors modulating the hypoxia-inducible factor (“HIF”) degradation pathway. Briefly, PHDs are responsible for hydroxylation of HIFα, a subunit of HIF, which initiates the pathway that eventually results in the degradation of HIFα by the proteasome. There are three subtypes of PHDs, including PHD1, PHD2, and PHD3. Inhibition of PHDs has been indicated as a promising therapy for the HIFα related disease, such as inflammatory epithelial disease, e.g., inflammatory bowel disease (IBD).

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 shows the disease activity index (DAI) score of Compound 29 and Mesalamine alone and in combination in IBD mouse model.

FIG. 2 shows colon Index of Compound 29 and Mesalamine alone and in combination in IBD mouse model.

FIG. 3 shows DAI score of Compound 29 and Cyclosporine A (CsA) alone and in combination with each other in IBD mouse model.

FIG. 4 shows colon Index of Compound 29 and Cyclosporine A (CsA) alone and in combination with each other in IBD mouse model.

FIG. 5 shows DAI score of Compound 29 and anti-TNFα antibody alone and in combination with each other in IBD mouse model.

FIG. 6 shows colon Index of Compound 29 and anti-TNFα antibody alone and in combination with each other in IBD mouse model.

### SUMMARY

Disclosed herein are methods of treating an inflammatory epithelial disease in a subject in need thereof, the method comprising the administering to the subject: a compound of Formula (I), or a pharmaceutically acceptable salt, or stereoisomer thereof:

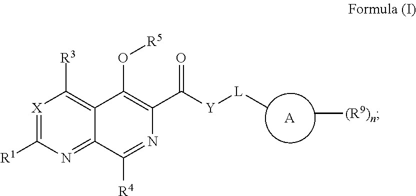

(b) an additional agent or a pharmaceutically acceptable salt thereof; wherein the combined amount of (a) and (b) is therapeutically effective for the treating of the inflammatory epithelial disease.

In some embodiments, the compound of Formula (I) is a compound of Table 1, or a pharmaceutically acceptable salt, or stereoisomer thereof. In some embodiments, the compound of Formula (I) is

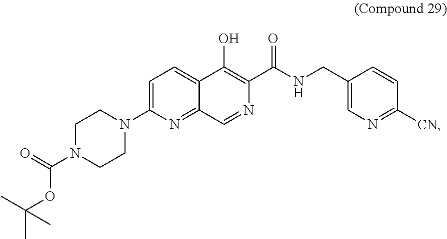 [SMILES: CC(C)(C)OC(=O)N1CCN(c2ccc3c(O)c(C(=O)NCc4ccc(C#N)nc4)ncc3n2)CC1]

or a pharmaceutically acceptable salt thereof, or stereoisomer thereof.

In some embodiments, the inflammatory epithelial disease is a disease affecting the respiratory tract, mucosa, skin, gastrointestinal tract, lining of major organs and endocrine glands, vascular tissue, or any combination thereof. In some embodiments, the inflammatory epithelial disease is inflammatory bowel disease. In some embodiments, the inflammatory epithelial disease is ulcerative colitis, Crohn's disease, collagenous colitis, lymphocytic colitis, ischemic colitis, diversion colitis, Behcet's syndrome, or indeterminate colitis.

In some embodiments, the additional agent is a 5-aminosalicylates (5-ASAs), anti-inflammatory agent, an ASK1 inhibitor, an alpha-fetoprotein modulator, an adenosine A3 receptor antagonist, an adrenomedullin ligand, an AKT1 gene inhibitor, an anti-CD28 inhibitor, an antibiotic, an antifungal, an ATPase inhibitor, a beta adrenoceptor antagonist, a BTK inhibitor, a beta-glucuronidase inhibitor, a bradykinin receptor modulator, a calcineurin inhibitor, a chaperonin binding immunoglobulin protein, a calcium channel inhibitor, a cathepsin S inhibitor, a CCR3 chemokine antagonist, a CD40 ligand receptor antagonist, a chemokine CXC ligand inhibitor, a CHST15 gene inhibitor, a collagen modulator, a CSF-1 antagonist, a cyclooxygenase inhibitor, a cytochrome P450 3A4 inhibitor, a carbohydrate metabolism modulator, a CCR9 chemokine antagonist, a CD233 modulator, a CD29 modulator, a CD3 antagonist, a CD4 antagonist, a CD40 ligand inhibitor, a CD40 gene inhibitor, a CX3CR1 chemokine modulator, a COT protein kinase inhibitor, an eotaxin ligand inhibitor, an EP4 prostanoid receptor agonist, an erythropoietin receptor agonist, an ecobiotic, an F1F0 ATP synthase modulator, a farnesoid X receptor (FXR and NR1H4) agonist or modulator, a fecal microbiota transplantation (FMT), a fractalkine ligand inhibitor, a free fatty acid receptor 2 antagonist, a GATA 3 transcription factor inhibitor, a glucagon-like peptide 2 agonist, a glucocorticoid receptor modulator, glucocorticoid receptor agonist, a guanylate cyclase receptor agonist, a histone deacetylase inhibitor, a histone deacetylase-6 inhibitor, an HLA class II antigen modulator, an immunosuppressant, an IL-12 antagonist, an IL-13 antagonist, an IL-23 antagonist, an IL-6 antagonist, an IL-6 receptor modulator, an IL-7 receptor modulator, an IL-7 antagonist, an IL-8 antagonist, an integrin alpha-4/beta-1 antagonist, an integrin alpha-4/beta-7 antagonist, an integrin alpha-E antagonist, an integrin antagonist, an integrin beta-7 antagonist, an interleukin ligand inhibitor, an interleukin-2 ligand, an interleukin receptor 17A antagonist, an interleukin-1 beta ligand, an interleukin-1 beta ligand modulator, an IRAK4 inhibitor, an ICAM1 gene inhibitor, an IL-1 beta ligand modulator, an IL-18 antagonist, an IL-22 agonist, an IL-23A inhibitor, an interleukin 1 like receptor 2 inhibitor, a JAK inhibitor, a JAK tyrosine kinase inhibitor, a Jak1 tyrosine kinase inhibitor, a Jak3 tyrosine kinase inhibitor, an LanC like protein 2 modulator, a lipoxygenase modulator, a lactoferrin stimulator, a leukocyte elastate inhibitor, a leukocyte proteinase-3 inhibitor, a MAdCAM inhibitor, a matrix metalloprotease inhibitor, a melanocortin agonist, a melanocortin MC1 agonist, a metalloprotease-9 inhibitor, a melanin concentrating hormone (MCH-1) antagonist, a microbiome modulator, a natriuretic peptide receptor C agonist, a neuregulin-4 ligand, an NKG2 D activating NK receptor antagonist, an NLRP3 inhibitor, a neuregulin-4 ligand, a nuclear factor kappa B inhibitor, an opioid receptor antagonist, an opioid receptor delta antagonist, an oxidoreductase inhibitor, an OX40 ligand inhibitor, a Pellino homolog 1 inhibitor, a P2X7 purinoceptor agonist, a PDE 4 inhibitor, a phagocytosis stimulating peptide modulator, a potassium channel inhibitor, a PPAR alpha agonist, a PPAR delta agonist, a PPAR gamma agonist, a protein fimH inhibitor, a P-selectin glycoprotein ligand-1 inhibitor, a Ret tyrosine kinase receptor inhibitor, an RNA polymerase inhibitor, a RIP-1 kinase inhibitor, a RIP-2 kinase inhibitor, a tissue transglutaminase inhibitor, a sphingosine-1-phosphate phosphatase-1 stimulator, a sphingosine-1-phosphate phosphatase modulator, a sphingosine-1-phosphate receptor-1 agonist, a sphingosine-1-phosphate receptor-1 antagonist, a sphingosine-1-phosphate receptor-1 modulator, a sphingosine-1-phosphate receptor-5 modulator, a STAT3 gene inhibitor, a stem cell antigen-1 inhibitor, a superoxide dismutase modulator, a superoxide dismutase stimulator, an SYK inhibitor, a TGF beta 1 ligand inhibitor, a thymulin agonist, a TLR antagonist, a TLR agonist, a TNF alpha ligand inhibitor, a TNF antagonist, a tumor necrosis factor superfamily member 14 modulator, a type II TNF receptor modulator, a Tpl 2 inhibitor, a TLR-3 antagonist, a TLR-4 antagonist, a TLR-8 inhibitor, a TLR-9 agonist, a TNF ligand inhibitor, a TNF alpha ligand modulator, a TPL-2 inhibitor, a tumor necrosis factor superfamily member 15 inhibitor, a Tyk2 tyrosine kinase inhibitor, a type I IL-1 receptor antagonist, a TrkA receptor antagonist, a vanilloid VR1 agonist, a zonulin inhibitor, or a pharmaceutically acceptable salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:
“oxo” refers to ═O.
“Carboxyl” refers to —COOH.
“Cyano” refers to —CN.
“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl[—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl(phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₅ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 Daltons, and more typically, up to about 500 Daltons.

The term “one or more” when referring to an optional substituent means that the subject group is optionally substituted with one, two, three, four, or more substituents. In some embodiments, the subject group is optionally substituted with one, two, three or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g., a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

### Compounds

Described herein are compounds of Formula (I), or a pharmaceutically acceptable salt, or stereoisomer thereof useful in the treatment of inflammatory epithelial disease such as inflammatory bowel disease (IBD).

Disclosed herein is a compound of Formula (I), or a pharmaceutically acceptable salt, or stereoisomer thereof:

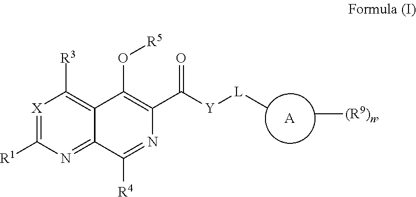

wherein:
R¹ is monocyclic heterocycloalkyl which is optionally and independently substituted;
X is N or CR²;
R² is hydrogen, fluoro, chloro, bromo, —CN, —NO₂, —OH, —OR—, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R³ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁴ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁵ is hydrogen, C₁-C₆alkyl, C₁-C₆haloalkyl, C₁-C₆hydroxyalkyl, C₁-C₆aminoalkyl, or C₁-C₆heteroalkyl; Y is —O—, —S—, or —NR⁶—;
R⁶ is hydrogen, C₁-C₆alkyl, C₁-C₆haloalkyl, C₁-C₆hydroxyalkyl, C₁-C₆aminoalkyl, or C₁-C₆heteroalkyl;
L is —(CR⁷R⁸)ₚ—;
each R⁷ and R⁸ are independently hydrogen, C₁-C₆alkyl, C₁-C₆haloalkyl, C₁-C₆hydroxyalkyl, C₁-C₆aminoalkyl, or C₁-C₆heteroalkyl;
or R⁷ and R⁸ on the same carbon are taken together to form a cycloalkyl or heterocycloalkyl; each optionally substituted with one or more R^{7a};
each R^{7a} is independently halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
p is 0-4;
Ring A is cycloalkyl, heterocycloalkyl, aryl, or heteroaryl;
each R⁹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR¹S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted;
n is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted;
or R^{c} and R^{d} are taken together with the atom to which they are attached to form an optionally substituted heterocycloalkyl.

Disclosed herein is a compound of Formula (I), or a pharmaceutically acceptable salt, or stereoisomer thereof:

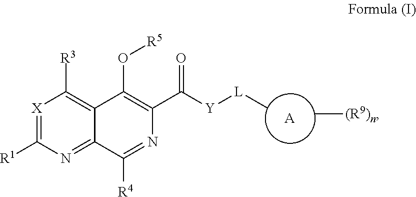

wherein: R¹ is monocyclic heterocycloalkyl optionally and independently substituted with one or more R^{1a}; each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{1a} on the same atom are taken together to form an oxo;
X is N or CR²;
R² is hydrogen, fluoro, chloro, bromo, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R³ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁴ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁵ is hydrogen, C₁-C₆alkyl, C₁-C₆haloalkyl, C₁-C₆hydroxyalkyl, C₁-C₅aminoalkyl, or C₁-C₆heteroalkyl;
Y is —O—, —S—, or —NR⁶—;
R⁶ is hydrogen, C₁-C₆alkyl, C₁-C₆haloalkyl, C₁-C₆hydroxyalkyl, C₁-C₆aminoalkyl, or C₁-C₆heteroalkyl;
L is —(CR⁷R⁸)ₚ—;
each R⁷ and R⁸ are independently hydrogen, C₁-C₆alkyl, C₁-C₆haloalkyl, C₁-C₆hydroxyalkyl, C₁-C₆aminoalkyl, or C₁-C₆heteroalkyl;
or R⁷ and R¹ on the same carbon are taken together to form a cycloalkyl or heterocycloalkyl; each optionally substituted with one or more R^{7a};
each R^{7a} is independently halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
p is 0-4;
Ring A is cycloalkyl, heterocycloalkyl, aryl, or heteroaryl;
each R⁹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{9a} on the same atom are taken together to form an oxo;
n is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; or
two R on the same atom are taken together to form an oxo.

In some embodiments of a compound of Formula (I), or a pharmaceutically acceptable salt, or stereoisomer thereof, X is N. In some embodiments of a compound of Formula (I), or a pharmaceutically acceptable salt, or stereoisomer thereof, X is CR².

In some embodiments of a compound of Formula (I), or a pharmaceutically acceptable salt, or stereoisomer thereof, R² is hydrogen, fluoro, or C₁-C₆alkyl. In some embodiments of a compound of Formula (I), or a pharmaceutically acceptable salt, or stereoisomer thereof, R² is hydrogen or C₁-C₆alkyl. In some embodiments of a compound of Formula (I), or a pharmaceutically acceptable salt, or stereoisomer thereof, R² is hydrogen.

In some embodiments of a compound of Formula (I), or a pharmaceutically acceptable salt, or stereoisomer thereof, R³ is hydrogen, halogen, C₁-C₆alkyl, or C₁-C₆haloalkyl. In some embodiments of a compound of Formula (I), or a pharmaceutically acceptable salt, or stereoisomer thereof, R³ is hydrogen, halogen, or C₁-C₆alkyl. In some embodiments of a compound of Formula (I), or a pharmaceutically acceptable salt, or stereoisomer thereof, R³ is hydrogen or C₁-C₆alkyl. In some embodiments of a compound of Formula (I), or a pharmaceutically acceptable salt, or stereoisomer thereof, R³ is hydrogen.

In some embodiments of a compound of Formula (I), or a pharmaceutically acceptable salt, or stereoisomer thereof, R⁴ is hydrogen, halogen, C₁-C₆alkyl, or C₁-C₆haloalkyl. In some embodiments of a compound of Formula (1), or a pharmaceutically acceptable salt, or stereoisomer thereof, R⁴ is hydrogen, halogen, or C₁-C₆alkyl. In some embodiments of a compound of Formula (I), or a pharmaceutically acceptable salt, or stereoisomer thereof, R⁴ is hydrogen or C₁-C₆alkyl. In some embodiments of a compound of Formula (I), or a pharmaceutically acceptable salt, or stereoisomer thereof, R⁴ is hydrogen.

In some embodiments of a compound of Formula (I), or a pharmaceutically acceptable salt, or stereoisomer thereof, R⁵ is hydrogen or C₁-C₆alkyl. In some embodiments of a compound of Formula (I), or a pharmaceutically acceptable salt, or stereoisomer thereof, R⁵ is C₁-C₆alkyl. In some embodiments of a compound of Formula (I), or a pharmaceutically acceptable salt, or stereoisomer thereof, R⁵ is hydrogen.

In some embodiments of a compound of Formula (I), or a pharmaceutically acceptable salt, or stereoisomer thereof,

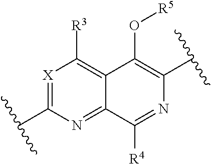

is

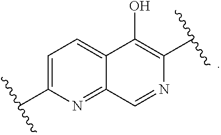 [SMILES: *c1ccc2c(O)c(*)ncc2n1]

In some embodiments of a compound of Formula (I), or a pharmaceutically acceptable salt, or stereoisomer thereof,

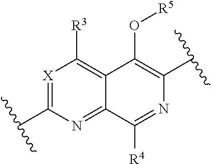

is

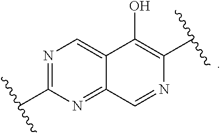 [SMILES: *c1ncc2c(O)c(*)ncc2n1]

In some embodiments of a compound of Formula (I), or a pharmaceutically acceptable salt, or stereoisomer thereof, Y is —O— or —NR⁶—. In some embodiments of a compound of Formula (I), or a pharmaceutically acceptable salt, or stereoisomer thereof, Y is —NR⁶—. In some embodiments of a compound of Formula (I), or a pharmaceutically acceptable salt, or stereoisomer thereof, Y is —O—. In some embodiments of a compound of Formula (I), or a pharmaceutically acceptable salt, or stereoisomer thereof, Y is —S—.

In some embodiments of a compound of Formula (I), or a pharmaceutically acceptable salt, or stereoisomer thereof, R⁶ is hydrogen or C₁-C₆alkyl. In some embodiments of a compound of Formula (I), or a pharmaceutically acceptable salt, or stereoisomer thereof, R⁶ is C₁-C₆alkyl. In some embodiments of a compound of Formula (I), or a pharmaceutically acceptable salt, or stereoisomer thereof, R⁶ is hydrogen.

In some embodiments of a compound of Formula (I), or a pharmaceutically acceptable salt, or stereoisomer thereof, p is 1-4. In some embodiments of a compound of Formula (I), or a pharmaceutically acceptable salt, or stereoisomer thereof, p is 1-3. In some embodiments of a compound of Formula (I), or a pharmaceutically acceptable salt, or stereoisomer thereof, p is 1 or 2. In some embodiments of a compound of Formula (I), or a pharmaceutically acceptable salt, or stereoisomer thereof, p is 1. In some embodiments of a compound of Formula (I), or a pharmaceutically acceptable salt, or stereoisomer thereof, p is 2. In some embodiments of a compound of Formula (I), or a pharmaceutically acceptable salt, or stereoisomer thereof, p is 3.

In some embodiments of a compound of Formula (I), or a pharmaceutically acceptable salt, or stereoisomer thereof, each R⁷ and R¹ are independently hydrogen, C₁-C₆alkyl, C₁-C₆haloalkyl, or C₁-C₆hydroxyalkyl; or R⁷ and R¹ on the same carbon are taken together to form a cycloalkyl or heterocycloalkyl. In some embodiments of a compound of Formula (I), or a pharmaceutically acceptable salt, or stereoisomer thereof, each R⁷ and R¹ are independently hydrogen, C₁-C₆alkyl, C₁-C₆haloalkyl, or C₁-C₆hydroxyalkyl. In some embodiments of a compound of Formula (I), or a pharmaceutically acceptable salt, or stereoisomer thereof, R⁷ and R⁸ on the same carbon are taken together to form a cycloalkyl or heterocycloalkyl. In some embodiments of a compound of Formula (I), or a pharmaceutically acceptable salt, or stereoisomer thereof, each R⁷ and R¹ are independently hydrogen or C₁-C₆alkyl. In some embodiments of a compound of Formula (I), or a pharmaceutically acceptable salt, or stereoisomer thereof, each R⁷ and R⁸ are hydrogen.

In some embodiments of a compound of Formula (I), or a pharmaceutically acceptable salt, or stereoisomer thereof, each R^{7a} is independently halogen, —CN, —OH, —OR^{a}, —NR^{c}R^{d}, C₁-C₆alkyl, or C₁-C₆haloalkyl. In some embodiments of a compound of Formula (I), or a pharmaceutically acceptable salt, or stereoisomer thereof, each R^{7a} is independently halogen, —OH, —OR^{a}, C₁-C₆alkyl.

In some embodiments of a compound of Formula (I), or a pharmaceutically acceptable salt, or stereoisomer thereof, Ring A is aryl or heteroaryl. In some embodiments of a compound of Formula (I), or a pharmaceutically acceptable salt, or stereoisomer thereof, Ring A is phenyl. In some embodiments of a compound of Formula (I), or a pharmaceutically acceptable salt, or stereoisomer thereof, Ring A is 5- or 6-membered heteroaryl. In some embodiments of a compound of Formula (I), or a pharmaceutically acceptable salt, or stereoisomer thereof, Ring A is 6-membered heteroaryl. In some embodiments of a compound of Formula (I), or a pharmaceutically acceptable salt, or stereoisomer thereof, Ring A is 6-membered pyridyl.

In some embodiments of a compound of Formula (I), or a pharmaceutically acceptable salt, or stereoisomer thereof, n is 1-3. In some embodiments of a compound of Formula (I), or a pharmaceutically acceptable salt, or stereoisomer thereof, n is 2-4. In some embodiments of a compound of Formula (I), or a pharmaceutically acceptable salt, or stereoisomer thereof, n is 2 or 3. In some embodiments of a compound of Formula (I), or a pharmaceutically acceptable salt, or stereoisomer thereof, n is 1 or 2. In some embodiments of a compound of Formula (I), or a pharmaceutically acceptable salt, or stereoisomer thereof, n is 0. In some embodiments of a compound of Formula (I), or a pharmaceutically acceptable salt, or stereoisomer thereof, n is 1. In some embodiments of a compound of Formula (I), or a pharmaceutically acceptable salt, or stereoisomer thereof, n is 2. In some embodiments of a compound of Formula (I), or a pharmaceutically acceptable salt, or stereoisomer thereof, n is 3.

In some embodiments of a compound of Formula (I), or a pharmaceutically acceptable salt, or stereoisomer thereof, each R⁹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (I), or a pharmaceutically acceptable salt, or stereoisomer thereof, each R⁹ is independently halogen, —CN, —OH, —OR^{a}, —NR^{c}R^{d}, —C(═O)OR^{b}, C₁-C₆alkyl, or C₁-C₆haloalkyl.

In some embodiments of a compound of Formula (I), or a pharmaceutically acceptable salt, or stereoisomer thereof, each R⁹ is independently halogen or —CN.

In some embodiments of a compound of Formula (I), or a pharmaceutically acceptable salt, or stereoisomer thereof, each R⁹ is —CN.

In some embodiments of a compound of Formula (I), or a pharmaceutically acceptable salt, or stereoisomer thereof, R¹ is monocyclic heterocycloalkyl independently substituted with one or more R^{1a}. In some embodiments, R¹ is independently substituted with 1, 2, 3, or 4 R^{1a}. In some embodiments, R¹ is independently substituted with 1 or 2 R^{1a}. In some embodiments, R¹ is substituted monocyclic heterocycloalkyl.

In some embodiments, R¹ is a 4 membered, optionally substituted monocyclic heterocycloalkyl. In some embodiments, R¹ is a 5 membered, optionally substituted monocyclic heterocycloalkyl. In some embodiments, R¹ is a 6 membered, optionally substituted monocyclic heterocycloalkyl. In some embodiments of a compound of Formula (I), or a pharmaceutically acceptable salt, or stereoisomer thereof, R¹ is attached to the rest of the fragment of formula (I) via a nitrogen atom of R¹. In some embodiments of a compound of Formula (I), or a pharmaceutically acceptable salt, or stereoisomer thereof, R¹ is

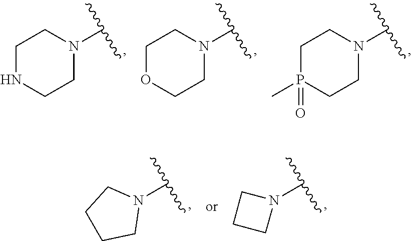

each of which is optionally substituted with one or more R^{1a}. In some embodiments, R¹ is

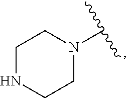 [SMILES: *N1CCNCC1]

which is optionally substituted with 1 or 2 R^{1a}. In some embodiments, R¹ is

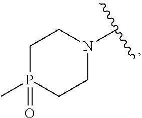 [SMILES: *N1CCP(C)(=O)CC1]

which is optionally substituted with 1 or 2 R^{1a}. In some embodiments, R¹ is

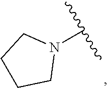 [SMILES: *N1CCCC1]

which is optionally substituted with 1 or 2 R^{1a}. In some embodiments, R¹ is

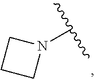 [SMILES: *N1CCC1]

which is optionally substituted with 1 or 2 R^{1a}

In some embodiments of a compound of Formula (I), or a pharmaceutically acceptable salt, or stereoisomer thereof, R¹ is azetidinyl, pyrrolidinyl, piperidinyl, piperazinyl, or morpholinyl, each optionally and independently substituted with one or more R^{1a}.

In some embodiments of a compound of Formula (I), or a pharmaceutically acceptable salt, or stereoisomer thereof, R¹ is piperidinyl optionally substituted with one or more R^{1a}.

In some embodiments, R¹ is monocyclic heterocycloalkyl optionally and independently substituted with 1 or 2 R^{1a}. In some embodiments, R¹ is 5-7 membered (e.g., 6 membered) monocyclic heterocycloalkyl optionally and independently substituted with 1 or 2 R^{1a}, and wherein the monocyclic heterocycloalkyl contains 1-3 ring nitrogen atoms.

In some embodiments of a compound of Formula (I), or a pharmaceutically acceptable salt, or stereoisomer thereof, R¹ is unsubstituted.

In some embodiments of a compound of Formula (I), or a pharmaceutically acceptable salt, or stereoisomer thereof, each R^{1a} is independently halogen, —CN, —OH, —OR^{a}, —NR^{c}R^{d}, —NR^{b}C(═O)NR^{c}R^{d}, —NR^{b}C(═O)R^{a}, —NR^{b}C(═O)OR^{b}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; or two R^{1a} on the same atom are taken together to form an oxo.

In some embodiments of a compound of Formula (I), or a pharmaceutically acceptable salt, or stereoisomer thereof, each R^{1a} is independently halogen, —CN, —OH, —OR^{a}, —NR^{c}R^{d}, —NR^{b}C(═O)R^{a}, —NR^{b}C(═O)OR^{b}, —C(═O)R^{a}, —C(—O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; or two R^{1a} on the same atom are taken together to form an oxo.

In some embodiments of a compound of Formula (I), or a pharmaceutically acceptable salt, or stereoisomer thereof, each R^{1a} is independently halogen, —CN, —OH, —OR^{a}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; or two R^{1a} on the same atom are taken together to form an oxo.

In some embodiments of a compound of Formula (I), or a pharmaceutically acceptable salt, or stereoisomer thereof, each R^{1a} is independently halogen, —OH, —OR^{a}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆heteroalkyl, or cycloalkyl; or two R^{1a} on the same atom are taken together to form an oxo.

In some embodiments of a compound of Formula (I), or a pharmaceutically acceptable salt, or stereoisomer thereof, each R^{1a} is independently C₁-C₆alkyl (e.g., methyl), C₁-C₆haloalkyl, or —C(═O)OR^{b} (e.g., —C(═O)O(C₁-C₆alkyl)).

In some embodiments of a compound of Formula (I), or a pharmaceutically acceptable salt, or stereoisomer thereof, R^{1a} is —C(═O)NR^{c}R^{d}. In some embodiments of a compound of Formula (I), or a pharmaceutically acceptable salt, or stereoisomer thereof, R^{1a} is —C(═O)NH₂. In some embodiments of a compound of Formula (I), or a pharmaceutically acceptable salt, or stereoisomer thereof, R^{1a} is

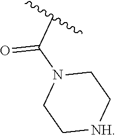 [SMILES: *C(=O)N1CCNCC1]

In some embodiments of a compound of Formula (I), or a pharmaceutically acceptable salt, or stereoisomer thereof, R¹ is

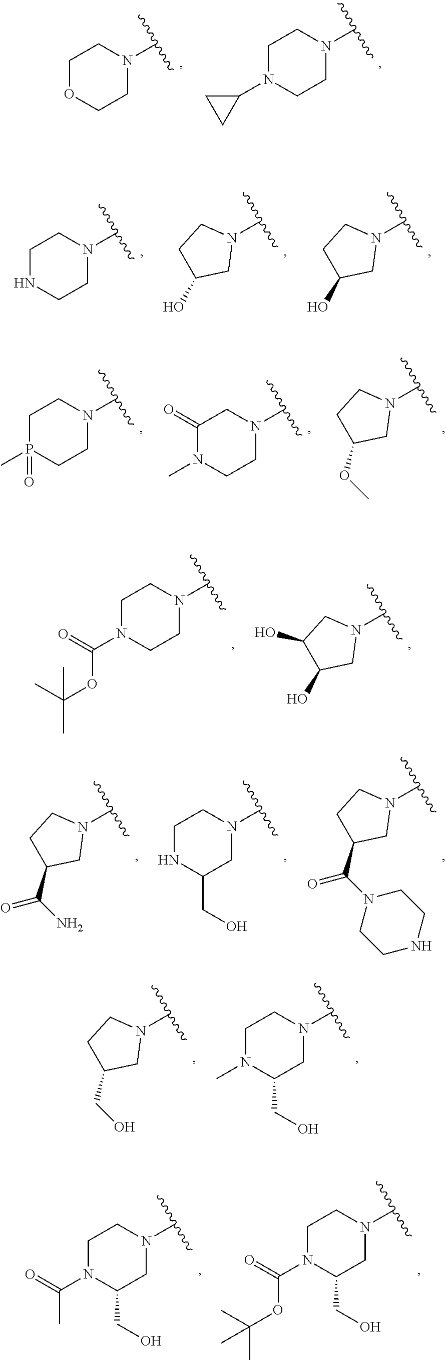

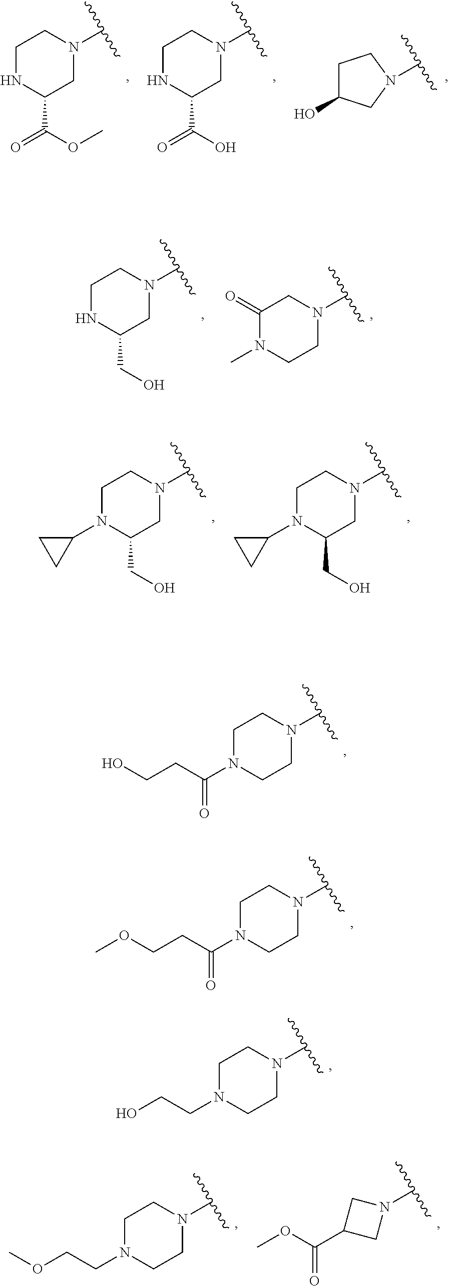

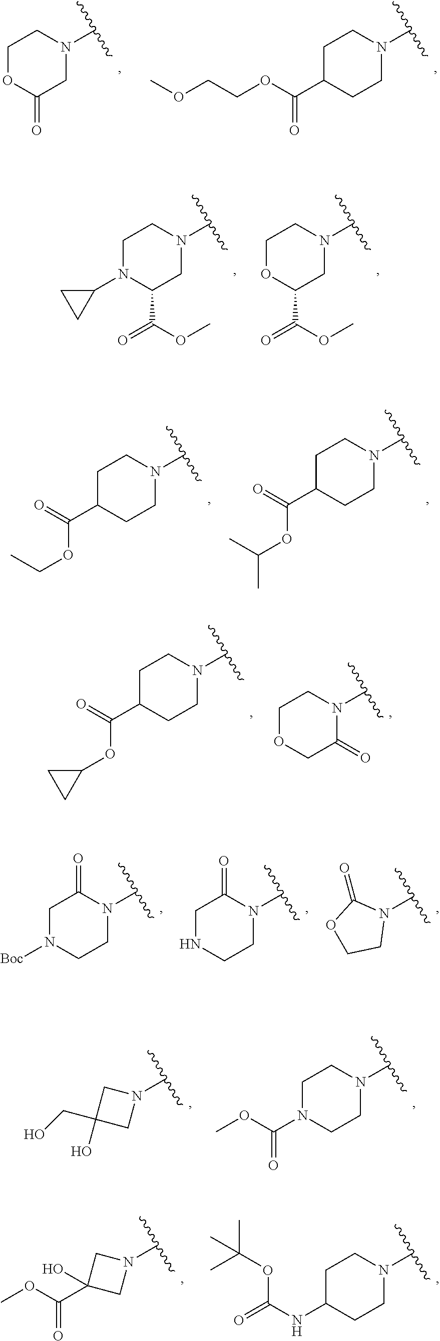

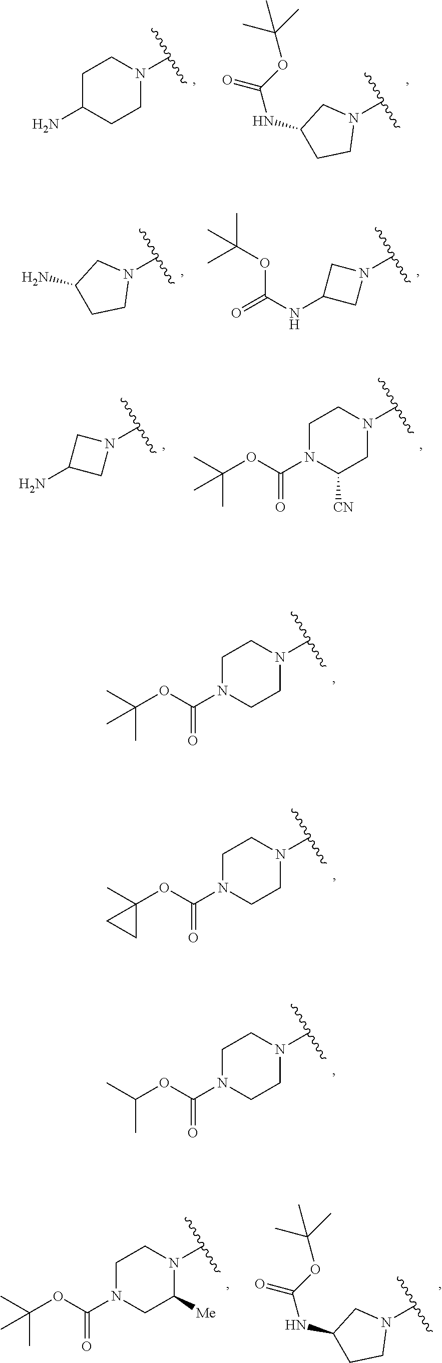

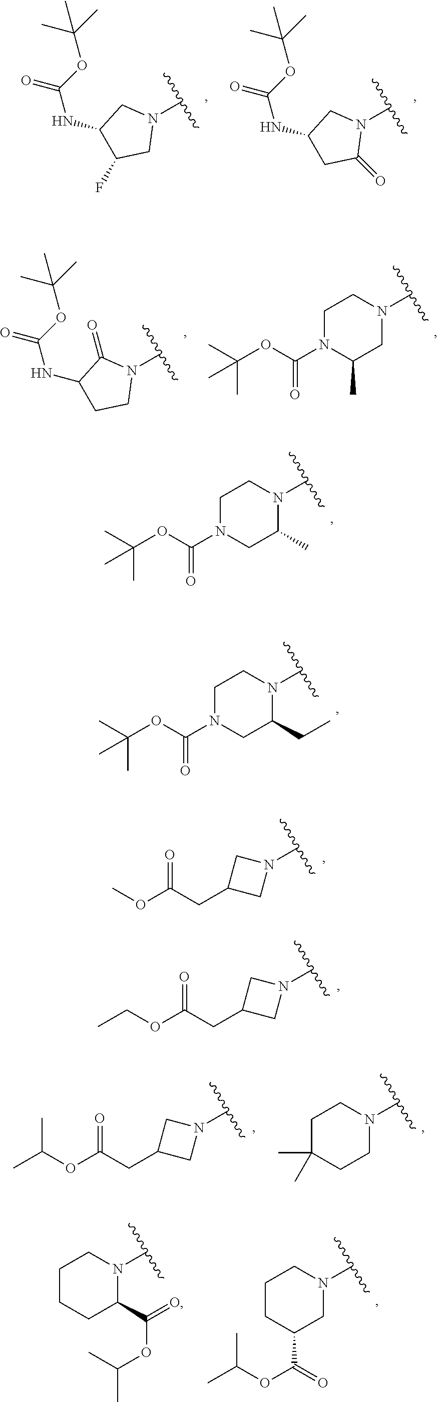

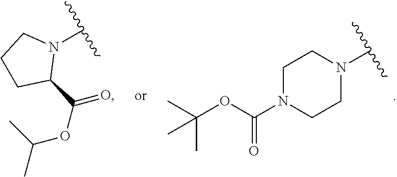 [SMILES: *N1CCC[C@@H]1C(=O)OC(C)C ; *N1CCN(C(=O)OC(C)(C)C)CC1]

In some embodiments of a compound of Formula (I), or a pharmaceutically acceptable salt, or stereoisomer thereof, R¹ is

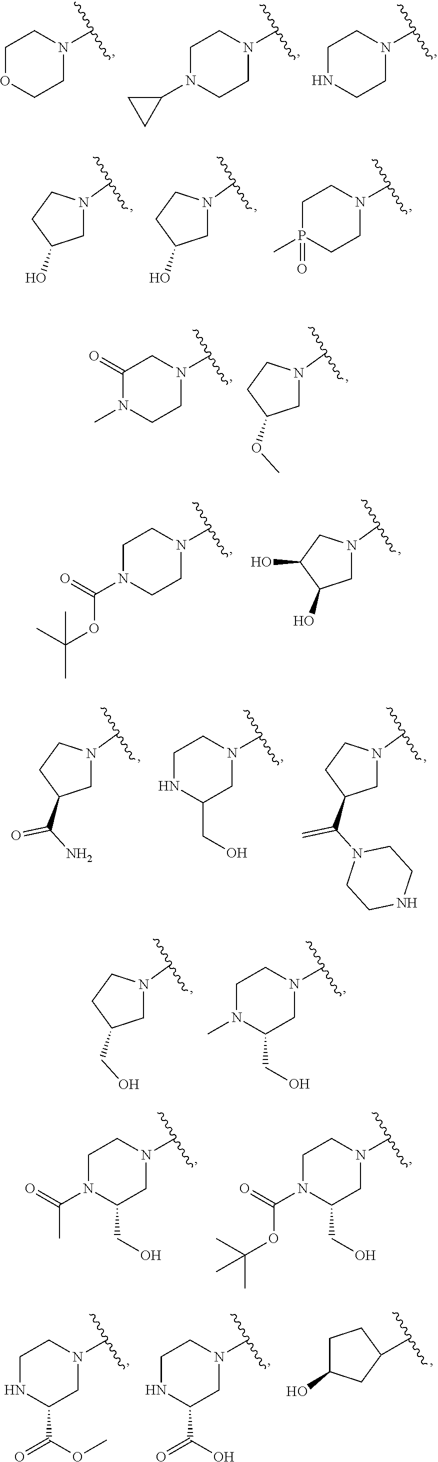

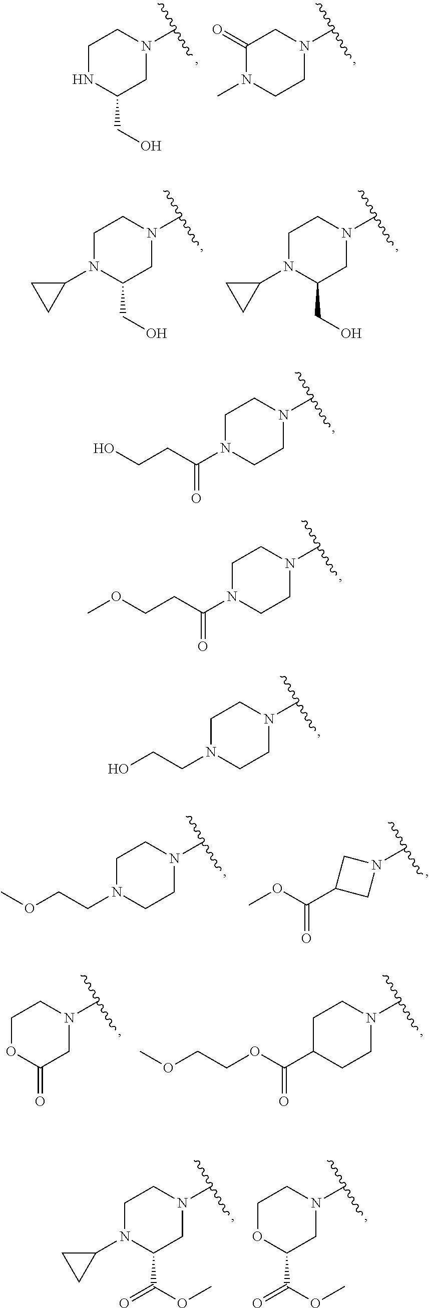

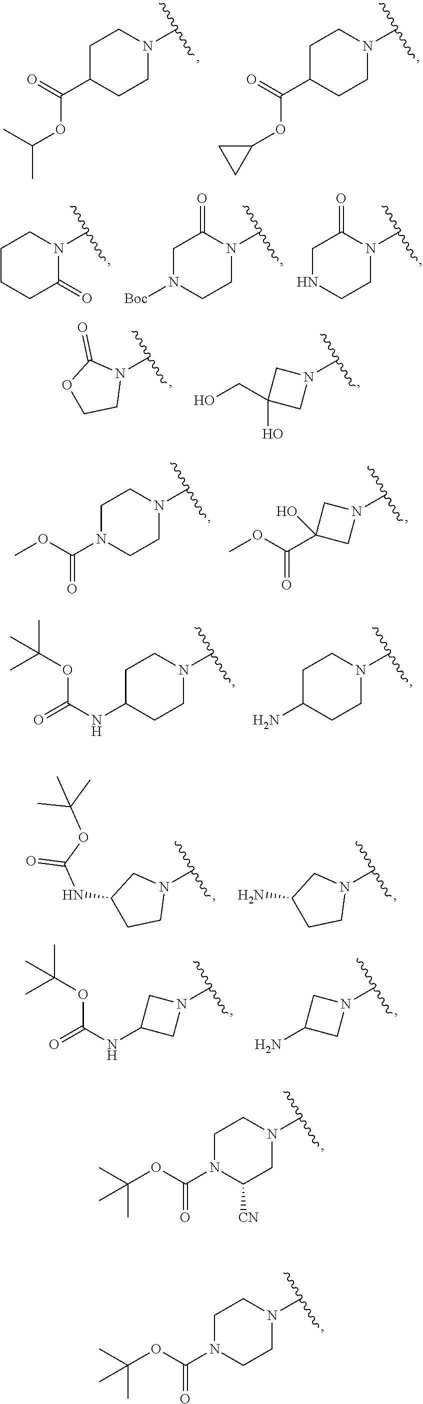

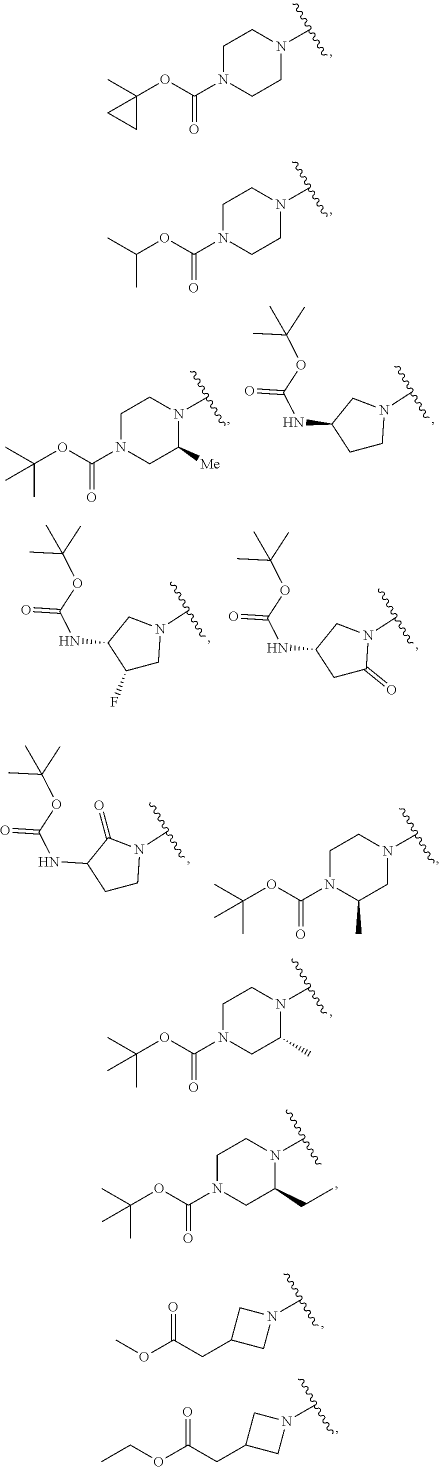

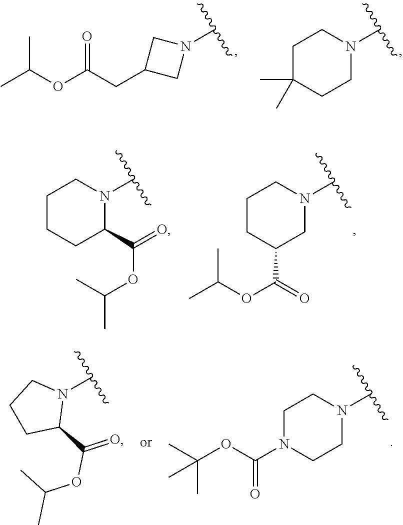

In some embodiments of a compound of Formula (1), or a pharmaceutically acceptable salt, or stereoisomer, thereof, R¹ is

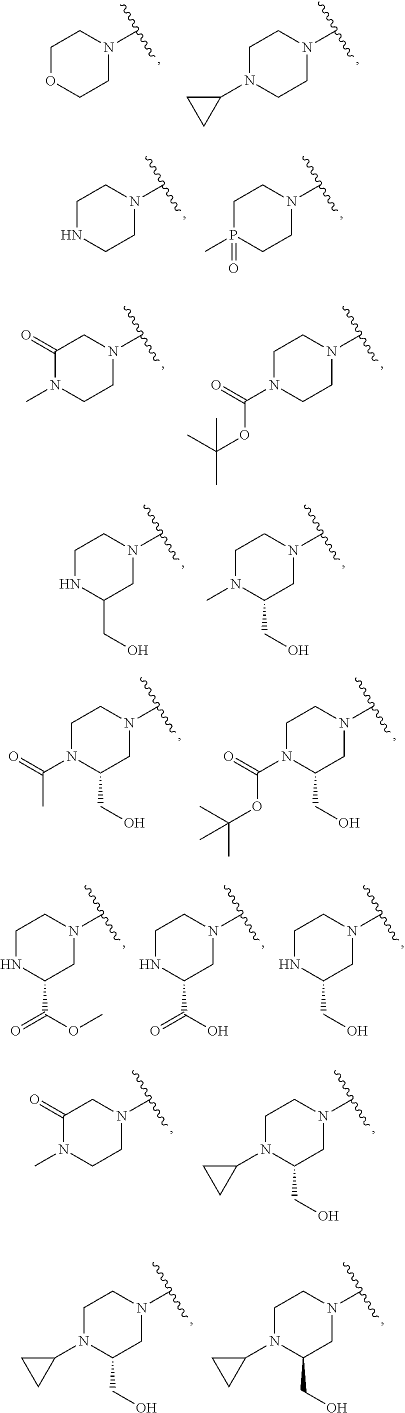

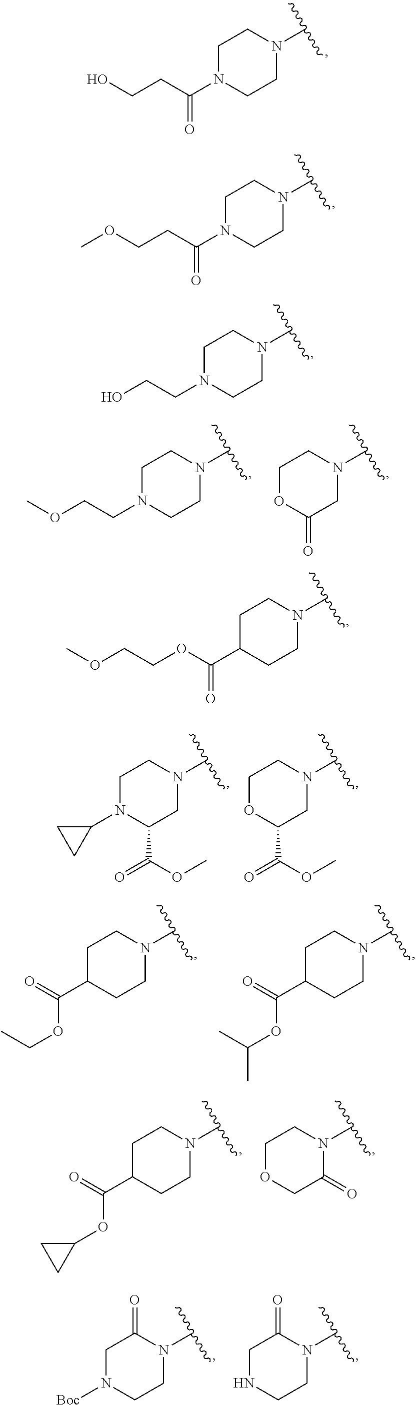

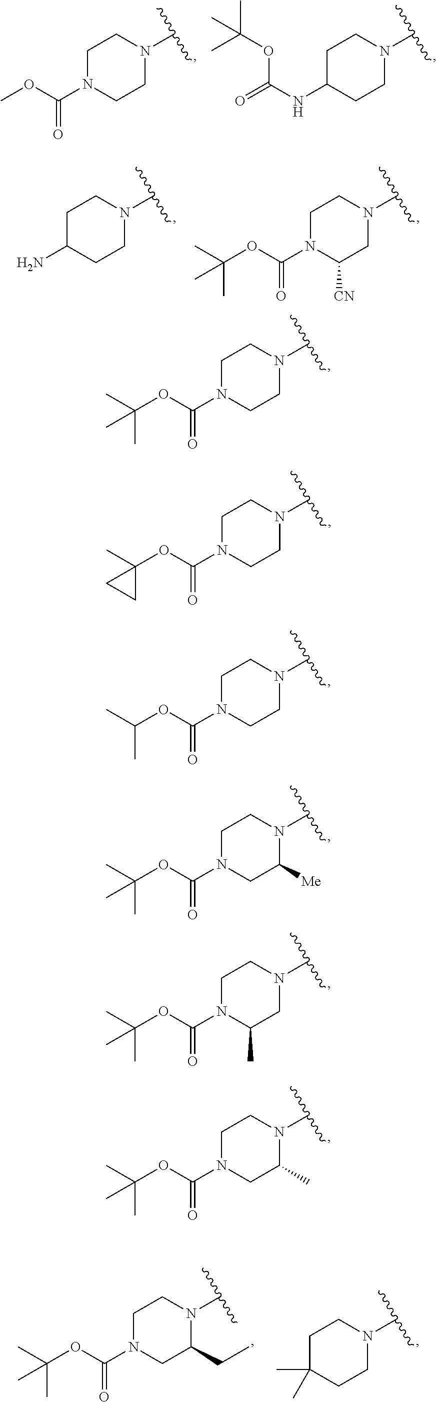

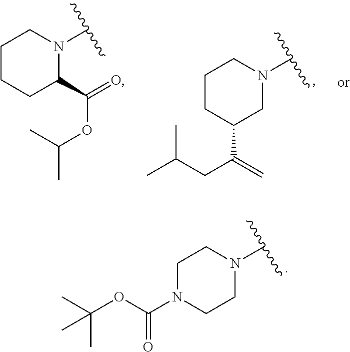

In some embodiments of a compound of Formula (I), or a pharmaceutically acceptable salt, or stereoisomer thereof, R¹ is

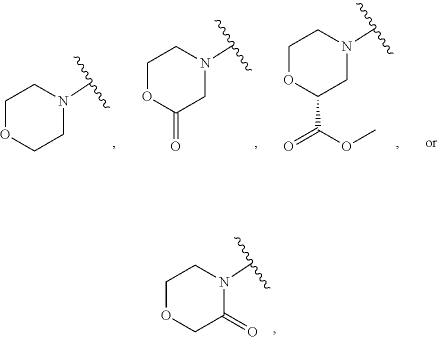 [SMILES: *N1CCOCC1 ; *N1CCOC(=O)C1 ; *N1CCO[C@@H](C(=O)OC)C1 ; *N1CCOCC1=O]

In some embodiments of a compound of Formula (1), or a pharmaceutically acceptable salt, or stereoisomer thereof, R¹ is

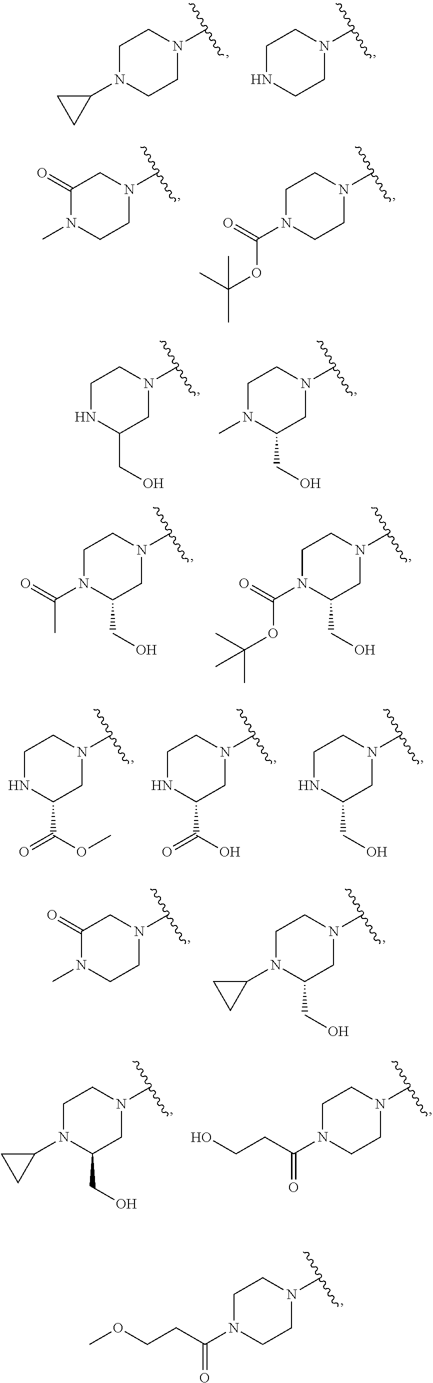

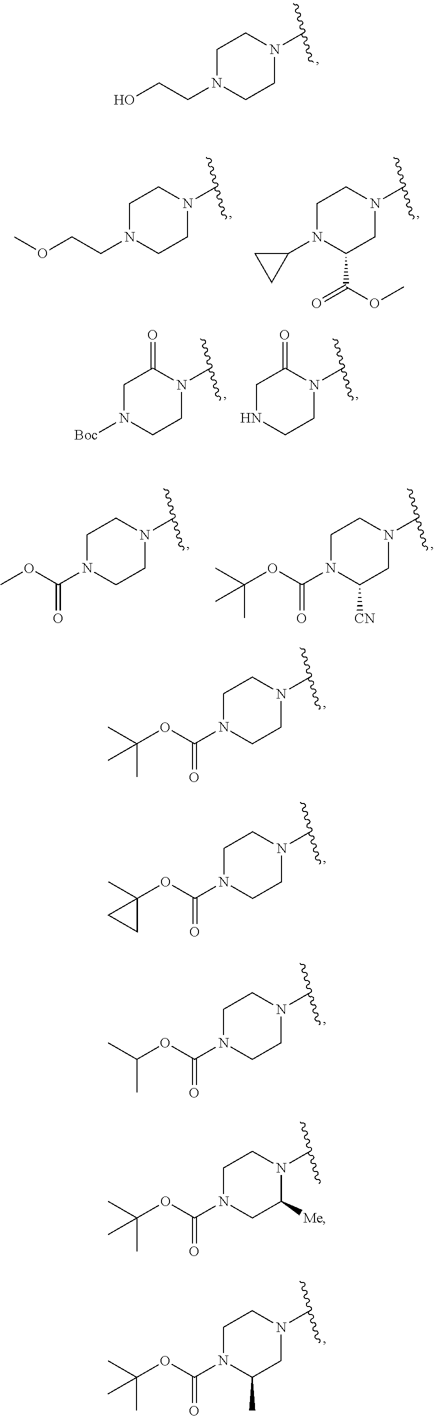

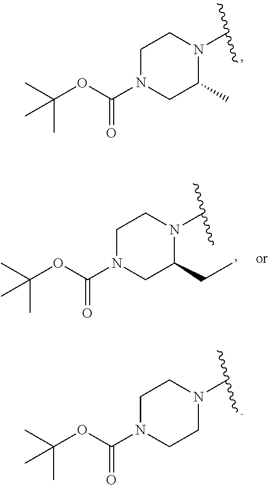

In some embodiments of a compound of Formula (I), or a pharmaceutically acceptable salt, or stereoisomer thereof, R¹ is

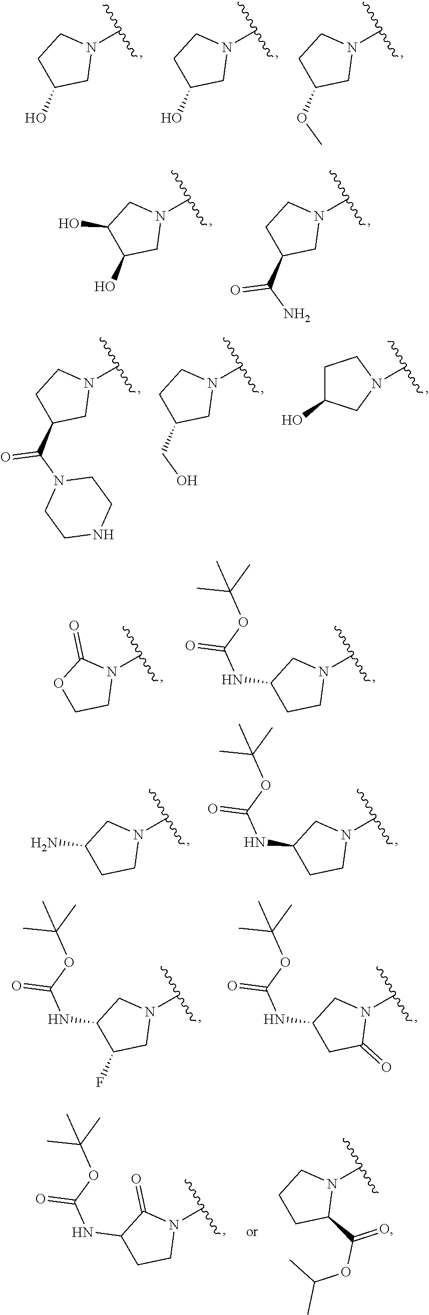

In some embodiments of a compound of Formula (I), or a pharmaceutically acceptable salt, or stereoisomer thereof, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound of Formula (I), or a pharmaceutically acceptable salt, or stereoisomer thereof, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound of Formula (I), or a pharmaceutically acceptable salt, or stereoisomer thereof, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (I), or a pharmaceutically acceptable salt, or stereoisomer thereof, each R^{a} is independently C₁-C₆alkyl, C₁-C₆haloalkyl, or cycloalkyl, heterocycloalkyl. In some embodiments of a compound of Formula (I), or a pharmaceutically acceptable salt, or stereoisomer thereof, each R^{a} is independently C₁-C₆alkyl or C₁-C₆haloalkyl. In some embodiments of a compound of Formula (I), or a pharmaceutically acceptable salt, or stereoisomer thereof, each R^{a} is independently C₁-C₆alkyl.

In some embodiments of a compound of Formula (I), or a pharmaceutically acceptable salt, or stereoisomer thereof, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound of Formula (I), or a pharmaceutically acceptable salt, or stereoisomer thereof, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound of Formula (I), or a pharmaceutically acceptable salt, or stereoisomer thereof, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (I), or a pharmaceutically acceptable salt, or stereoisomer thereof, each R^{b} is independently hydrogen, C₁-C₆alkyl, C₁-C₆haloalkyl, or cycloalkyl, heterocycloalkyl. In some embodiments of a compound of Formula (I), or a pharmaceutically acceptable salt, or stereoisomer thereof, each R^{b} is independently hydrogen, C₁-C₆alkyl or C₁-C₆haloalkyl. In some embodiments of a compound of Formula (I), or a pharmaceutically acceptable salt, or stereoisomer thereof, each R^{b} is independently hydrogen or C₁-C₆alkyl. In some embodiments of a compound of Formula (I), or a pharmaceutically acceptable salt, or stereoisomer thereof, each R^{b} is hydrogen. In some embodiments of a compound of Formula (I), or a pharmaceutically acceptable salt, or stereoisomer thereof, each R^{b} is independently C₁-C₆alkyl.

In some embodiments of a compound of Formula (I), or a pharmaceutically acceptable salt, or stereoisomer thereof, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound of Formula (I), or a pharmaceutically acceptable salt, or stereoisomer thereof, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound of Formula (I), or a pharmaceutically acceptable salt, or stereoisomer thereof, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (I), or a pharmaceutically acceptable salt, or stereoisomer thereof, each R^{c} and R^{d} are independently hydrogen, C₁-C₆alkyl, C₁-C₆haloalkyl, or cycloalkyl, heterocycloalkyl. In some embodiments of a compound of Formula (I), or a pharmaceutically acceptable salt, or stereoisomer thereof, each R^{c} and R^{d} are independently hydrogen, C₁-C₆alkyl or C₁-C₆haloalkyl. In some embodiments of a compound of Formula (I), or a pharmaceutically acceptable salt, or stereoisomer thereof, each R^{c} and R^{d} are independently hydrogen or C₁-C₆alkyl. In some embodiments of a compound of Formula (I), or a pharmaceutically acceptable salt, or stereoisomer thereof, each R^{c} and R^{d} are hydrogen. In some embodiments of a compound of Formula (I), or a pharmaceutically acceptable salt, or stereoisomer thereof, each R^{c} and R^{d} are independently C₁-C₆alkyl. In some embodiments of a compound of Formula (I), or a pharmaceutically acceptable salt, or stereoisomer thereof, R^{e} is hydrogen, C₁-C₆hydroxyalkyl, C₁-C₆alkyl, C₁-C₆aminoalkyl, or C₁-C₆haloalkyl. In some embodiments of a compound of Formula (I), or a pharmaceutically acceptable salt, or stereoisomer thereof, R^{d} is hydrogen, C₁-C₆hydroxyalkyl, C₁-C₆alkyl, C₁-C₆haloalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl). In some embodiments of a compound of Formula (I), or a pharmaceutically acceptable salt, or stereoisomer thereof, R^{d} is —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, —CH₂CH₂N(CH₃)₂, —CH₂CH₂OH, or —CH₂CH₂NHC(═O)O-t-butyl.

In some embodiments of a compound of Formula (I), or a pharmaceutically acceptable salt, or stereoisomer thereof, R^{c} and R^{d} are taken together with the atom to which they are attached to form a heterocycloalkyl optionally substituted with one or more R.

In some embodiments of a compound of Formula (I), or a pharmaceutically acceptable salt, or stereoisomer thereof, each R is independently halogen, —CN, —OH, —OC₁-C₆alkyl, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, or C₁-C₆haloalkyl. In some embodiments of a compound of Formula (I), or a pharmaceutically acceptable salt, or stereoisomer thereof, each R is independently halogen, —CN, —OH, —OC₁-C₆alkyl, —NH₂, —C(═O)C₁-C₆alkyl, —C(═O)OH, —C(═O)OC₁-C₆alkyl, —C(═O)NH₂, C₁-C₆alkyl, or C₁-C₆haloalkyl. In some embodiments of a compound of Formula (I), or a pharmaceutically acceptable salt, or stereoisomer thereof, each R is independently halogen, —CN, —OH, —OC₁-C₆alkyl, —NH₂, C₁-C₆alkyl, or C₁-C₆haloalkyl.

In some embodiments of a compound disclosed herein, each R¹, R⁹, R^{a}, R^{b}, R^{e}, R^{d}, the heterocycloalkyl formed when R⁷ and R¹ are taken together, and the heterocycloalkyl formed when R^{c} and R^{d} are taken together, is optionally and independently substituted with one, two, three, or four substituents as defined herein. In some embodiments of a compound disclosed herein, each R¹, R⁹, R^{a}, R^{b}, R^{c}, R^{d}, the heterocycloalkyl formed when R⁷ and R¹ are taken together, and the heterocycloalkyl formed when R^{c} and R^{d} are taken together, is optionally and independently substituted with one, two, or three substituents as defined herein. In some embodiments of a compound disclosed herein, each R¹, R⁹, R^{a}, R^{b}, R^{e}, R^{d}, the heterocycloalkyl formed when R⁷ and R⁸ are taken together, and the heterocycloalkyl formed when R^{c} and R^{d} are taken together, is optionally and independently substituted with one or two substituents as defined herein. In some embodiments of a compound disclosed herein, each R¹, R⁹, R^{a}, R^{b}, R^{e}, R^{d}, the heterocycloalkyl formed when R⁷ and R⁸ are taken together, and the heterocycloalkyl formed when R^{c} and R^{d} are taken together, is optionally and independently substituted with one substituent as defined herein.

In some embodiments of a compound disclosed herein, the abundance of deuterium in each of R, R¹, R^{1a}, R², R³, R⁴, R⁵, R⁶, R⁷, R^{7a}, R⁸, R⁹, R⁹, R^{a}, R^{b}, R^{c}, and/or R^{d} is independently at least 1%, at least 10%, at least 20%, at least 30%, at least 40%, at least 50%, at least 60%, at least 70%, at least 80%, at least 90%, or 100% of a total number of hydrogen and deuterium.

In some embodiments of a compound disclosed herein, one or more of R, R¹, R¹, R², R³, R⁴, R⁵, R⁶, R⁷, R^{7a}, R⁸, R⁹, R^{9a}, R^{a}, R^{b}, R^{e}, and/or R^{d} groups comprise deuterium at a percentage higher than the natural abundance of deuterium.

In some embodiments of a compound disclosed herein, one or more hydrogens are replaced with one or more deuteriums in one or more of the following groups R, R¹, R^{1a}, R², R³, R⁴, R⁵, R⁶, R⁷, R^{7a}, R⁸, R⁹, R^{9a}, R^{b}, R^{c} and R^{d}.

In some embodiments of a compound disclosed herein, one or more hydrogens of Ring A are replaced with one or more deuteriums.

Any combination of the groups described above for the various variables is contemplated herein. Throughout the specification, groups and substituents thereof arc chosen by one skilled in the field to provide stable moieties and compounds.

In some embodiments the compound of Formula (I), or a pharmaceutically acceptable salt, or stereoisomer thereof, is one of the compounds in Table 1.

TABLE 1

| Compound | |
|---|---|
| ID | Structure |
| 3 | 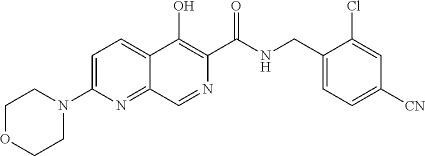 [SMILES: N#Cc1ccc(CNC(=O)c2ncc3nc(N4CCOCC4)ccc3c2O)c(Cl)c1] |
| 4 | 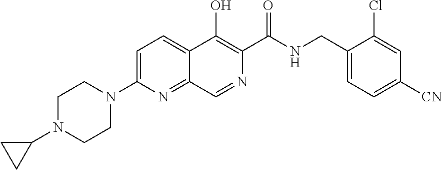 [SMILES: N#Cc1ccc(CNC(=O)c2ncc3nc(N4CCN(C5CC5)CC4)ccc3c2O)c(Cl)c1] |
| 5 | 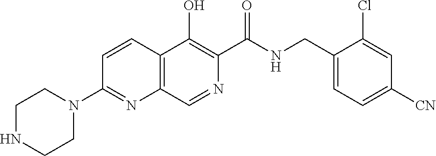 [SMILES: N#Cc1ccc(CNC(=O)c2ncc3nc(N4CCNCC4)ccc3c2O)c(Cl)c1] |
| 6 | 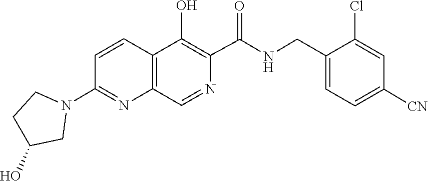 [SMILES: N#Cc1ccc(CNC(=O)c2ncc3nc(N4CC[C@@H](O)C4)ccc3c2O)c(Cl)c1] |
| 7 | 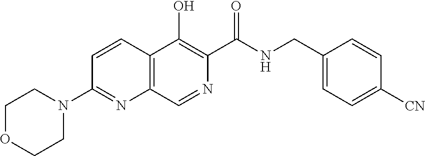 [SMILES: N#Cc1ccc(CNC(=O)c2ncc3nc(N4CCOCC4)ccc3c2O)cc1] |
| 9 | 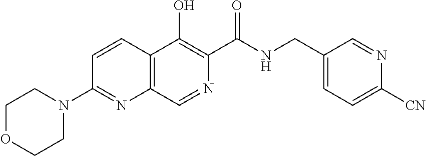 [SMILES: N#Cc1ccc(CNC(=O)c2ncc3nc(N4CCOCC4)ccc3c2O)cn1] |
| 10 | 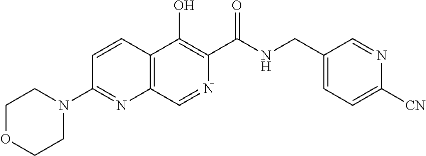 [SMILES: N#Cc1ccc(CNC(=O)c2ncc3nc(N4CCOCC4)ccc3c2O)cn1] |
| 15 | 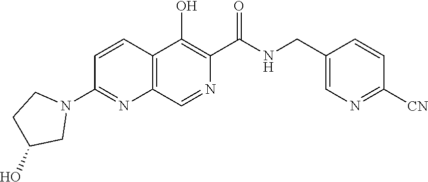 [SMILES: N#Cc1ccc(CNC(=O)c2ncc3nc(N4CC[C@@H](O)C4)ccc3c2O)cn1] |
| 16 | 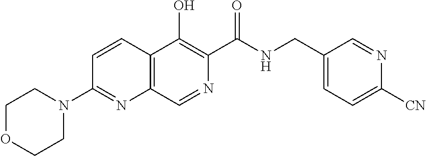 [SMILES: N#Cc1ccc(CNC(=O)c2ncc3nc(N4CCOCC4)ccc3c2O)cn1] |
| 17 | 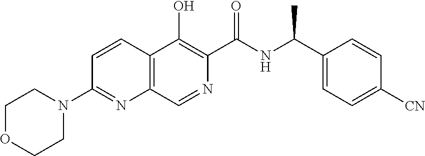 [SMILES: C[C@H](NC(=O)c1ncc2nc(N3CCOCC3)ccc2c1O)c1ccc(C#N)cc1] |
| 18 | 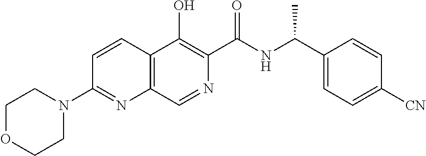 [SMILES: C[C@@H](NC(=O)c1ncc2nc(N3CCOCC3)ccc2c1O)c1ccc(C#N)cc1] |
| 19 | 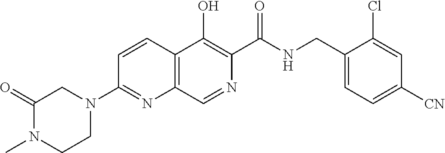 [SMILES: CN1CCN(c2ccc3c(O)c(C(=O)NCc4ccc(C#N)cc4Cl)ncc3n2)CC1=O] |
| 20 | 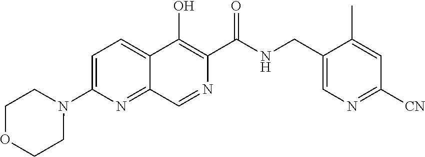 [SMILES: Cc1cc(C#N)ncc1CNC(=O)c1ncc2nc(N3CCOCC3)ccc2c1O] |
| 21 | 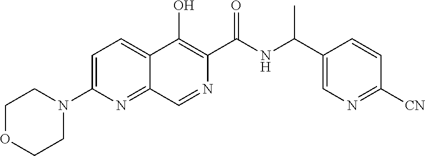 [SMILES: CC(NC(=O)c1ncc2nc(N3CCOCC3)ccc2c1O)c1ccc(C#N)nc1] |
| 22 | 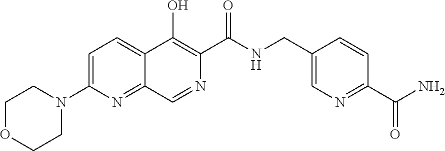 [SMILES: NC(=O)c1ccc(CNC(=O)c2ncc3nc(N4CCOCC4)ccc3c2O)cn1] |
| 23 | 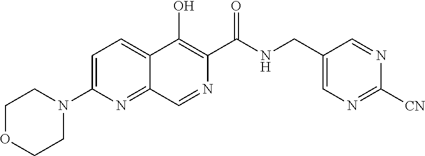 [SMILES: N#Cc1ncc(CNC(=O)c2ncc3nc(N4CCOCC4)ccc3c2O)cn1] |
| 24 | 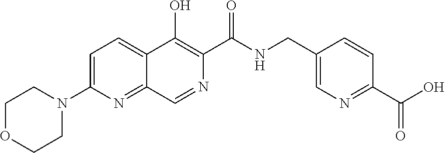 [SMILES: O=C(O)c1ccc(CNC(=O)c2ncc3nc(N4CCOCC4)ccc3c2O)cn1] |
| 26 | 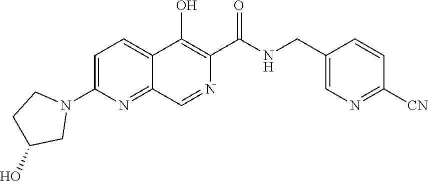 [SMILES: N#Cc1ccc(CNC(=O)c2ncc3nc(N4CC[C@@H](O)C4)ccc3c2O)cn1] |
| 27 | 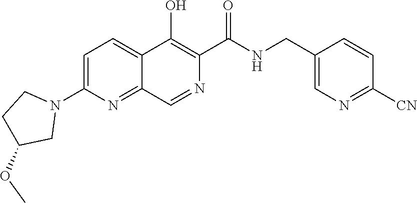 [SMILES: CO[C@@H]1CCN(c2ccc3c(O)c(C(=O)NCc4ccc(C#N)nc4)ncc3n2)C1] |
| 28 | 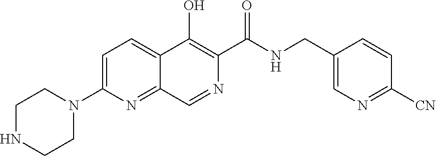 [SMILES: N#Cc1ccc(CNC(=O)c2ncc3nc(N4CCNCC4)ccc3c2O)cn1] |
| 29 | 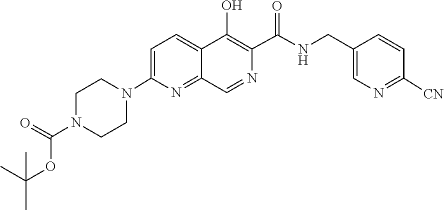 [SMILES: CC(C)(C)OC(=O)N1CCN(c2ccc3c(O)c(C(=O)NCc4ccc(C#N)nc4)ncc3n2)CC1] |
| 31 | 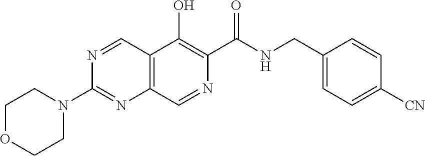 [SMILES: N#Cc1ccc(CNC(=O)c2ncc3nc(N4CCOCC4)ncc3c2O)cc1] |
| 32 | 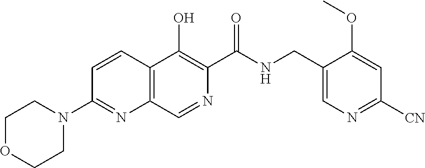 [SMILES: COc1cc(C#N)ncc1CNC(=O)c1ncc2nc(N3CCOCC3)ccc2c1O] |
| 33 | 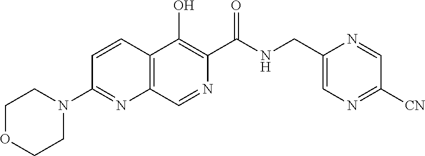 [SMILES: N#Cc1cnc(CNC(=O)c2ncc3nc(N4CCOCC4)ccc3c2O)cn1] |
| 34 | 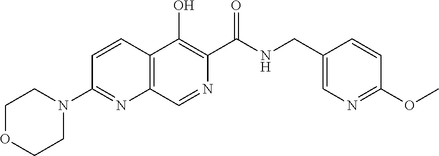 [SMILES: COc1ccc(CNC(=O)c2ncc3nc(N4CCOCC4)ccc3c2O)cn1] |
| 35 | 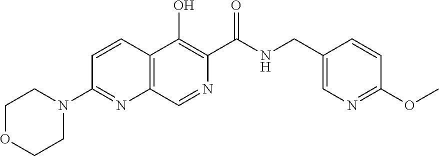 [SMILES: COc1ccc(CNC(=O)c2ncc3nc(N4CCOCC4)ccc3c2O)cn1] |
| 36 | 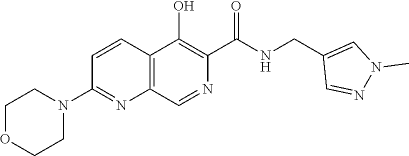 [SMILES: Cn1cc(CNC(=O)c2ncc3nc(N4CCOCC4)ccc3c2O)cn1] |
| 37 | 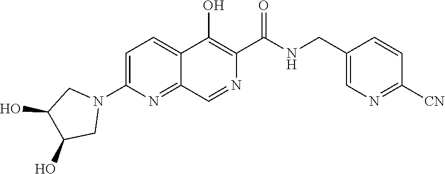 [SMILES: N#Cc1ccc(CNC(=O)c2ncc3nc(N4C[C@@H](O)[C@@H](O)C4)ccc3c2O)cn1] |
| 38 | 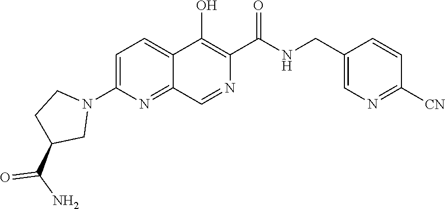 [SMILES: N#Cc1ccc(CNC(=O)c2ncc3nc(N4CC[C@H](C(N)=O)C4)ccc3c2O)cn1] |
| 39 | 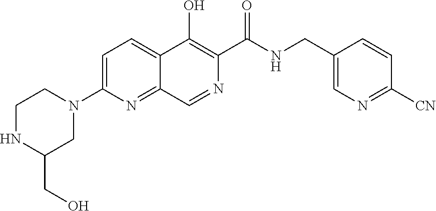 [SMILES: N#Cc1ccc(CNC(=O)c2ncc3nc(N4CCNC(CO)C4)ccc3c2O)cn1] |
| 40 | 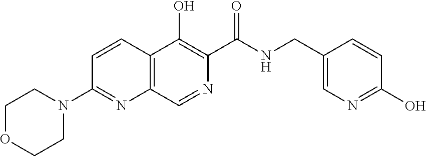 [SMILES: O=C(NCc1ccc(O)nc1)c1ncc2nc(N3CCOCC3)ccc2c1O] |
| 41 | 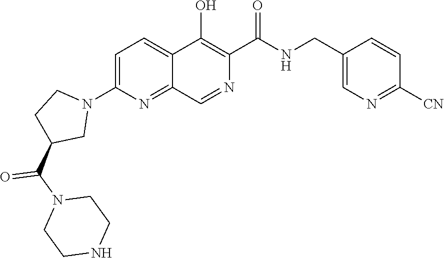 [SMILES: N#Cc1ccc(CNC(=O)c2ncc3nc(N4CC[C@H](C(=O)N5CCNCC5)C4)ccc3c2O)cn1] |
| 42 | 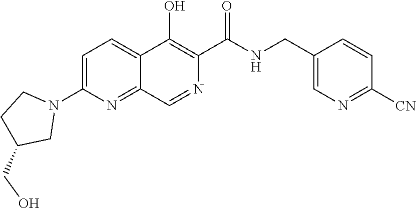 [SMILES: N#Cc1ccc(CNC(=O)c2ncc3nc(N4CC[C@@H](CO)C4)ccc3c2O)cn1] |
| 45 | 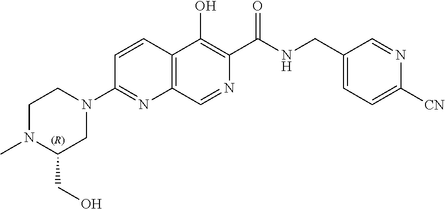 |
| 46 | 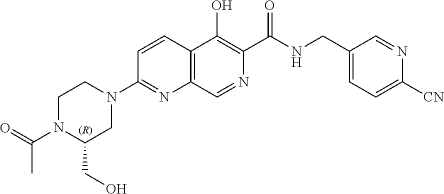 |
| 47 | 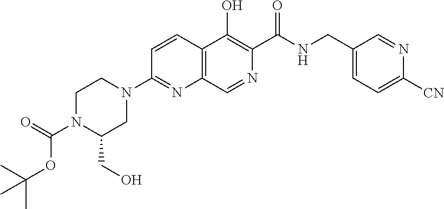 [SMILES: CC(C)(C)OC(=O)N1CCN(c2ccc3c(O)c(C(=O)NCc4ccc(C#N)nc4)ncc3n2)C[C@@H]1CO] |
| 48 | 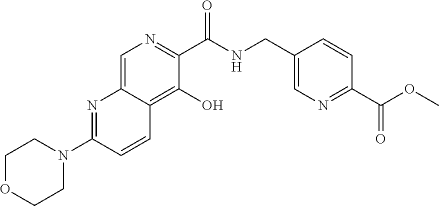 [SMILES: COC(=O)c1ccc(CNC(=O)c2ncc3nc(N4CCOCC4)ccc3c2O)cn1] |
| 50 | 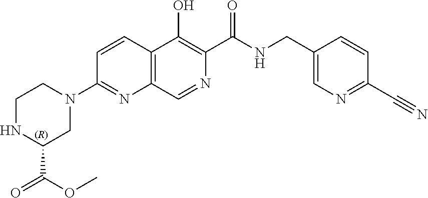 |
| 51 | 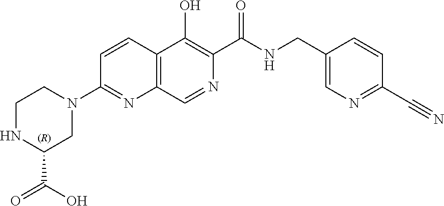 |
| 57 | 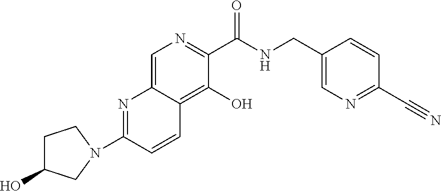 [SMILES: N#Cc1ccc(CNC(=O)c2ncc3nc(N4CC[C@H](O)C4)ccc3c2O)cn1] |
| 59 | 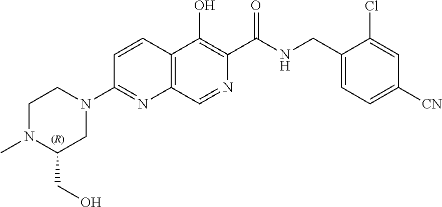 |
| 60 | 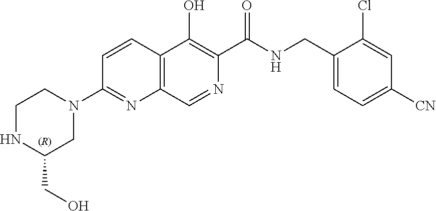 |
| 63 | 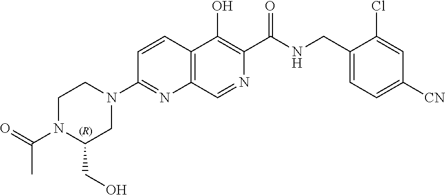 |
| 64 | 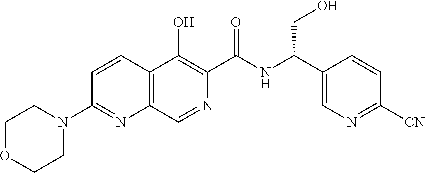 [SMILES: N#Cc1ccc([C@@H](CO)NC(=O)c2ncc3nc(N4CCOCC4)ccc3c2O)cn1] |
| 65 | 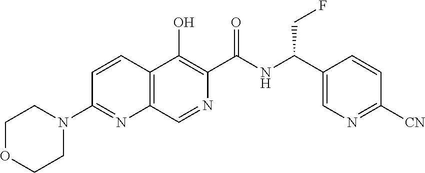 [SMILES: N#Cc1ccc([C@@H](CF)NC(=O)c2ncc3nc(N4CCOCC4)ccc3c2O)cn1] |
| 66 | 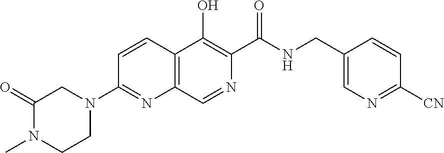 [SMILES: CN1CCN(c2ccc3c(O)c(C(=O)NCc4ccc(C#N)nc4)ncc3n2)CC1=O] |
| 70 | 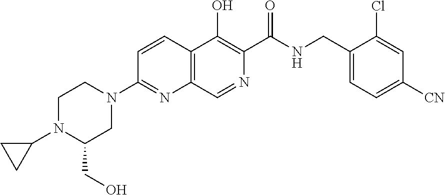 [SMILES: N#Cc1ccc(CNC(=O)c2ncc3nc(N4CCN(C5CC5)[C@@H](CO)C4)ccc3c2O)c(Cl)c1] |
| 71 | 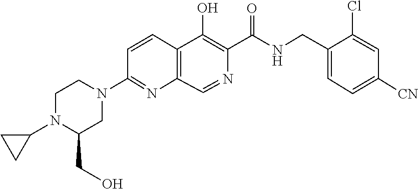 [SMILES: N#Cc1ccc(CNC(=O)c2ncc3nc(N4CCN(C5CC5)[C@H](CO)C4)ccc3c2O)c(Cl)c1] |
| 72 | 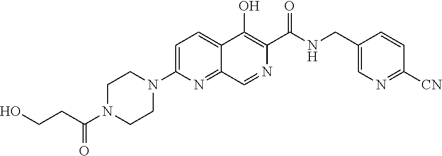 [SMILES: N#Cc1ccc(CNC(=O)c2ncc3nc(N4CCN(C(=O)CCO)CC4)ccc3c2O)cn1] |
| 73 | 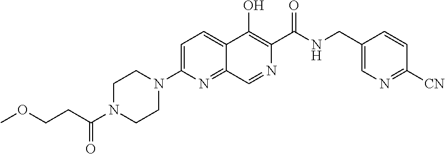 [SMILES: COCCC(=O)N1CCN(c2ccc3c(O)c(C(=O)NCc4ccc(C#N)nc4)ncc3n2)CC1] |
| 74 | 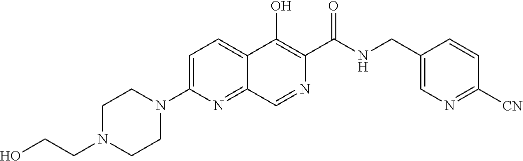 [SMILES: N#Cc1ccc(CNC(=O)c2ncc3nc(N4CCN(CCO)CC4)ccc3c2O)cn1] |
| 75 | 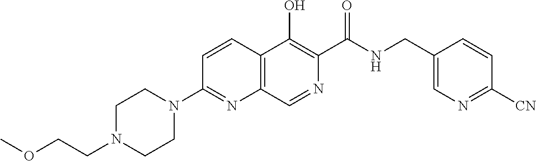 [SMILES: COCCN1CCN(c2ccc3c(O)c(C(=O)NCc4ccc(C#N)nc4)ncc3n2)CC1] |
| 84 | 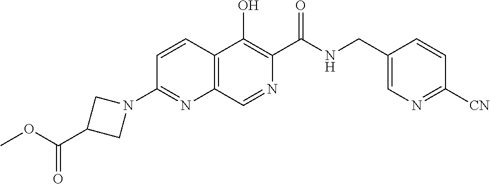 [SMILES: COC(=O)C1CN(c2ccc3c(O)c(C(=O)NCc4ccc(C#N)nc4)ncc3n2)C1] |
| 85 | 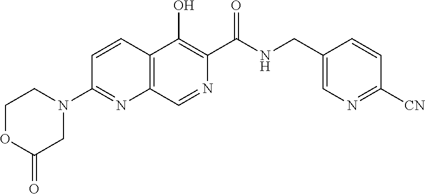 [SMILES: N#Cc1ccc(CNC(=O)c2ncc3nc(N4CCOC(=O)C4)ccc3c2O)cn1] |
| 86 | 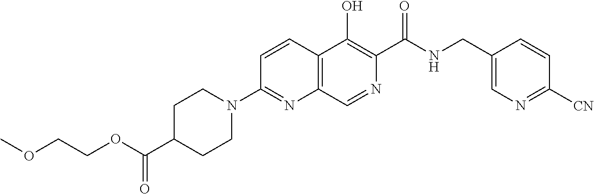 [SMILES: COCCOC(=O)C1CCN(c2ccc3c(O)c(C(=O)NCc4ccc(C#N)nc4)ncc3n2)CC1] |
| 89 | 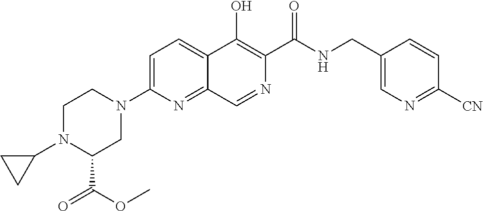 [SMILES: COC(=O)[C@H]1CN(c2ccc3c(O)c(C(=O)NCc4ccc(C#N)nc4)ncc3n2)CCN1C1CC1] |
| 92 | 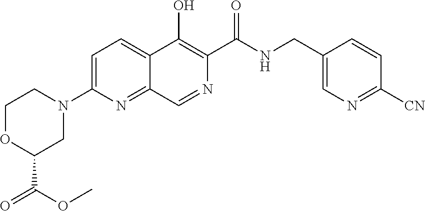 [SMILES: COC(=O)[C@H]1CN(c2ccc3c(O)c(C(=O)NCc4ccc(C#N)nc4)ncc3n2)CCO1] |
| 94 | 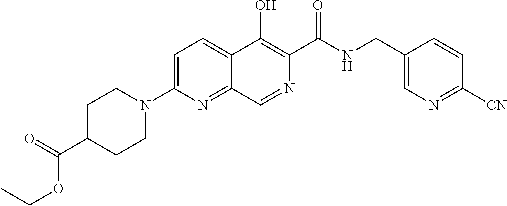 [SMILES: CCOC(=O)C1CCN(c2ccc3c(O)c(C(=O)NCc4ccc(C#N)nc4)ncc3n2)CC1] |
| 95 | 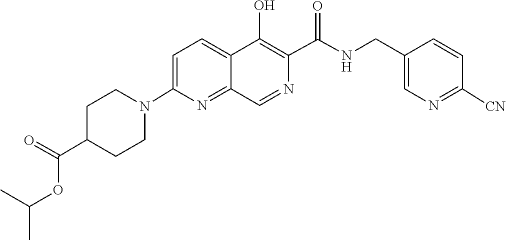 [SMILES: CC(C)OC(=O)C1CCN(c2ccc3c(O)c(C(=O)NCc4ccc(C#N)nc4)ncc3n2)CC1] |
| 96 | 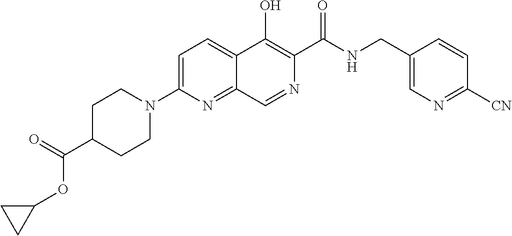 [SMILES: N#Cc1ccc(CNC(=O)c2ncc3nc(N4CCC(C(=O)OC5CC5)CC4)ccc3c2O)cn1] |
| 100 | 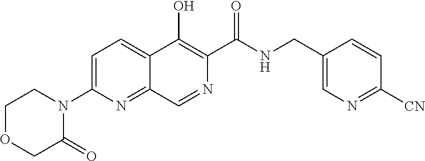 [SMILES: N#Cc1ccc(CNC(=O)c2ncc3nc(N4CCOCC4=O)ccc3c2O)cn1] |
| 101 | 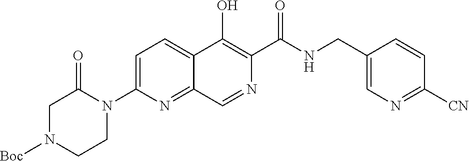 [SMILES: CC(C)(C)OC(=O)N1CCN(c2ccc3c(O)c(C(=O)NCc4ccc(C#N)nc4)ncc3n2)C(=O)C1] |
| 102 | 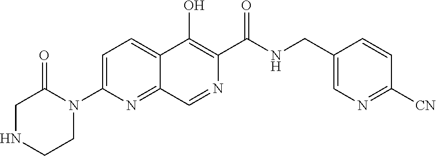 [SMILES: N#Cc1ccc(CNC(=O)c2ncc3nc(N4CCNCC4=O)ccc3c2O)cn1] |
| 103 | 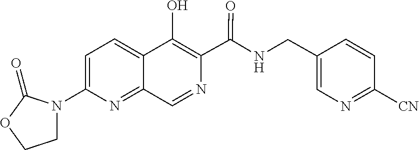 [SMILES: N#Cc1ccc(CNC(=O)c2ncc3nc(N4CCOC4=O)ccc3c2O)cn1] |
| 112 | 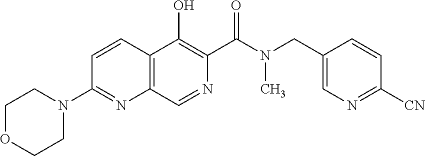 [SMILES: CN(Cc1ccc(C#N)nc1)C(=O)c1ncc2nc(N3CCOCC3)ccc2c1O] |
| 113 | 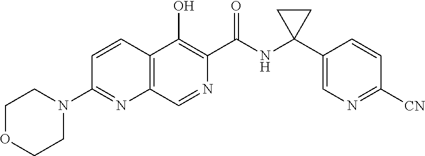 [SMILES: N#Cc1ccc(C2(NC(=O)c3ncc4nc(N5CCOCC5)ccc4c3O)CC2)cn1] |
| 114 | 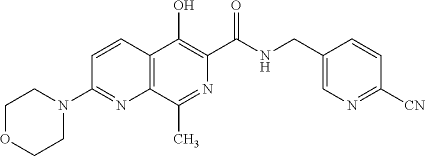 [SMILES: Cc1nc(C(=O)NCc2ccc(C#N)nc2)c(O)c2ccc(N3CCOCC3)nc12] |
| 119 | 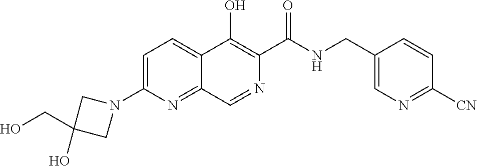 [SMILES: N#Cc1ccc(CNC(=O)c2ncc3nc(N4CC(O)(CO)C4)ccc3c2O)cn1] |
| 120 | 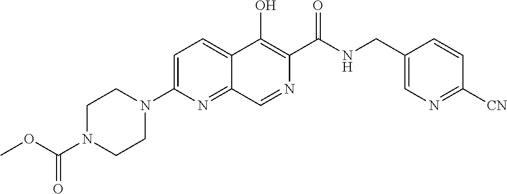 [SMILES: COC(=O)N1CCN(c2ccc3c(O)c(C(=O)NCc4ccc(C#N)nc4)ncc3n2)CC1] |
| 121 | 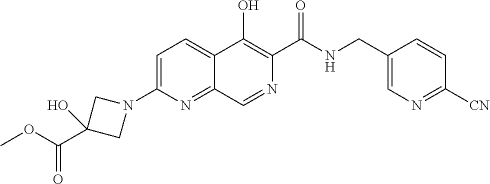 [SMILES: COC(=O)C1(O)CN(c2ccc3c(O)c(C(=O)NCc4ccc(C#N)nc4)ncc3n2)C1] |
| 123 | 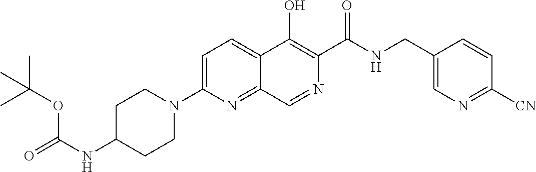 [SMILES: CC(C)(C)OC(=O)NC1CCN(c2ccc3c(O)c(C(=O)NCc4ccc(C#N)nc4)ncc3n2)CC1] |
| 124 | 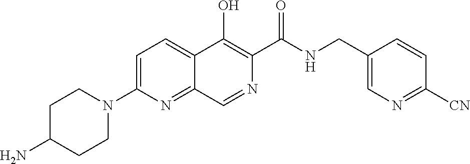 [SMILES: N#Cc1ccc(CNC(=O)c2ncc3nc(N4CCC(N)CC4)ccc3c2O)cn1] |
| 125 | 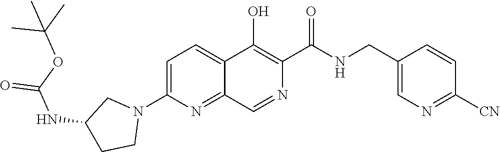 [SMILES: CC(C)(C)OC(=O)N[C@H]1CCN(c2ccc3c(O)c(C(=O)NCc4ccc(C#N)nc4)ncc3n2)C1] |
| 126 | 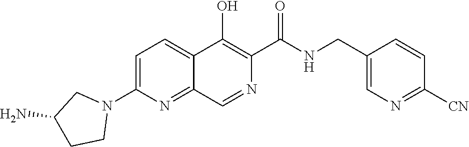 [SMILES: N#Cc1ccc(CNC(=O)c2ncc3nc(N4CC[C@H](N)C4)ccc3c2O)cn1] |
| 127 | 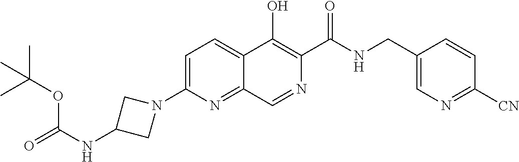 [SMILES: CC(C)(C)OC(=O)NC1CN(c2ccc3c(O)c(C(=O)NCc4ccc(C#N)nc4)ncc3n2)C1] |
| 128 | 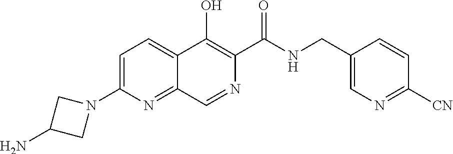 [SMILES: N#Cc1ccc(CNC(=O)c2ncc3nc(N4CC(N)C4)ccc3c2O)cn1] |
| 132 | 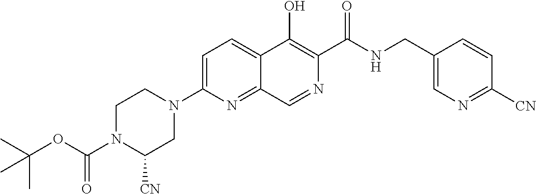 [SMILES: CC(C)(C)OC(=O)N1CCN(c2ccc3c(O)c(C(=O)NCc4ccc(C#N)nc4)ncc3n2)C[C@@H]1C#N] |
| 133 | 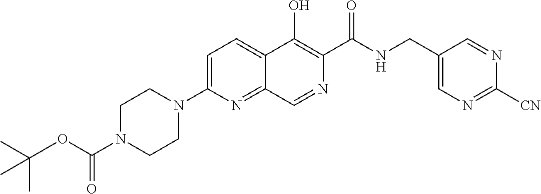 [SMILES: CC(C)(C)OC(=O)N1CCN(c2ccc3c(O)c(C(=O)NCc4cnc(C#N)nc4)ncc3n2)CC1] |
| 134 | 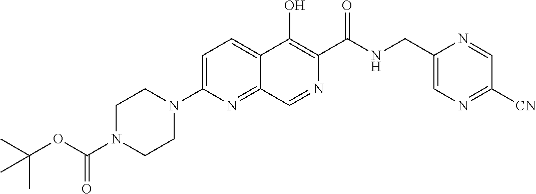 [SMILES: CC(C)(C)OC(=O)N1CCN(c2ccc3c(O)c(C(=O)NCc4cnc(C#N)cn4)ncc3n2)CC1] |
| 135 | 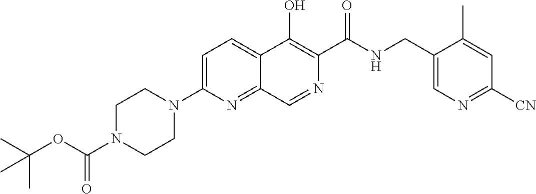 [SMILES: Cc1cc(C#N)ncc1CNC(=O)c1ncc2nc(N3CCN(C(=O)OC(C)(C)C)CC3)ccc2c1O] |
| 136 | 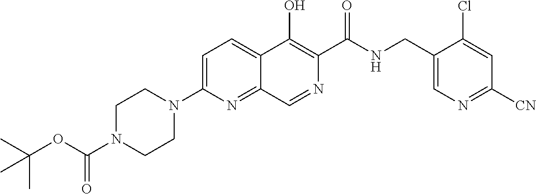 [SMILES: CC(C)(C)OC(=O)N1CCN(c2ccc3c(O)c(C(=O)NCc4cnc(C#N)cc4Cl)ncc3n2)CC1] |
| 137 | 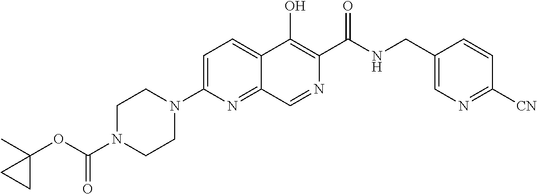 [SMILES: CC1(OC(=O)N2CCN(c3ccc4c(O)c(C(=O)NCc5ccc(C#N)nc5)ncc4n3)CC2)CC1] |
| 138 | 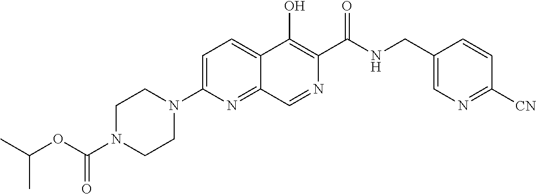 [SMILES: CC(C)OC(=O)N1CCN(c2ccc3c(O)c(C(=O)NCc4ccc(C#N)nc4)ncc3n2)CC1] |
| 139 | 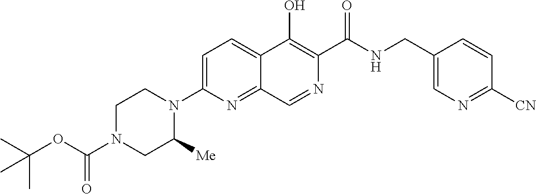 [SMILES: C[C@H]1CN(C(=O)OC(C)(C)C)CCN1c1ccc2c(O)c(C(=O)NCc3ccc(C#N)nc3)ncc2n1] |
| 140 | 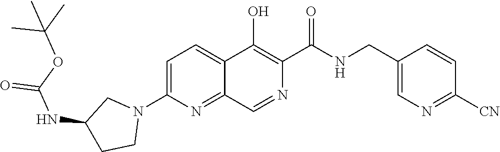 [SMILES: CC(C)(C)OC(=O)N[C@@H]1CCN(c2ccc3c(O)c(C(=O)NCc4ccc(C#N)nc4)ncc3n2)C1] |
| 142 | 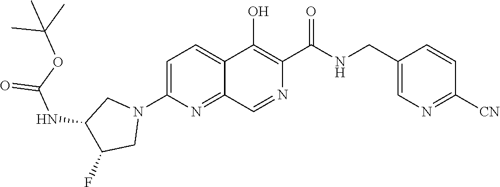 [SMILES: CC(C)(C)OC(=O)N[C@@H]1CN(c2ccc3c(O)c(C(=O)NCc4ccc(C#N)nc4)ncc3n2)C[C@@H]1F] |
| 143 | 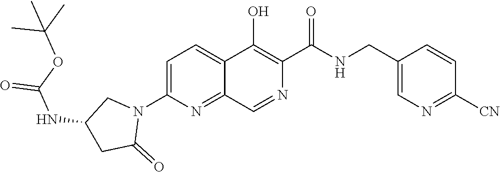 [SMILES: CC(C)(C)OC(=O)N[C@H]1CC(=O)N(c2ccc3c(O)c(C(=O)NCc4ccc(C#N)nc4)ncc3n2)C1] |
| 144 | 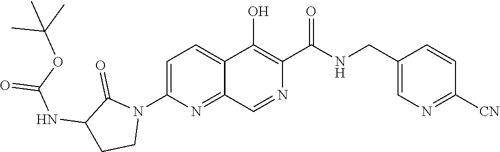 [SMILES: CC(C)(C)OC(=O)NC1CCN(c2ccc3c(O)c(C(=O)NCc4ccc(C#N)nc4)ncc3n2)C1=O] |
| 149 | 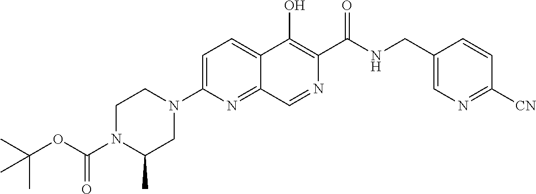 [SMILES: C[C@@H]1CN(c2ccc3c(O)c(C(=O)NCc4ccc(C#N)nc4)ncc3n2)CCN1C(=O)OC(C)(C)C] |
| 150 | 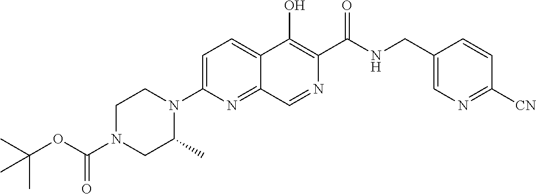 [SMILES: C[C@@H]1CN(C(=O)OC(C)(C)C)CCN1c1ccc2c(O)c(C(=O)NCc3ccc(C#N)nc3)ncc2n1] |
| 151 | 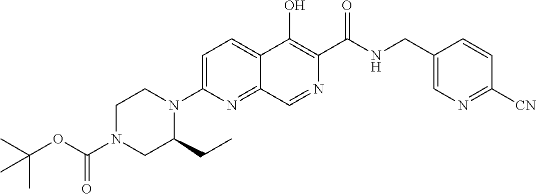 [SMILES: CC[C@H]1CN(C(=O)OC(C)(C)C)CCN1c1ccc2c(O)c(C(=O)NCc3ccc(C#N)nc3)ncc2n1] |
| 152 | 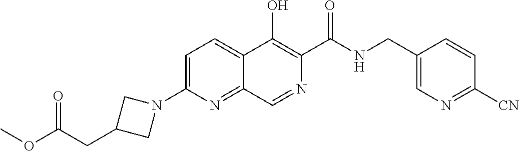 [SMILES: COC(=O)CC1CN(c2ccc3c(O)c(C(=O)NCc4ccc(C#N)nc4)ncc3n2)C1] |
| 153 | 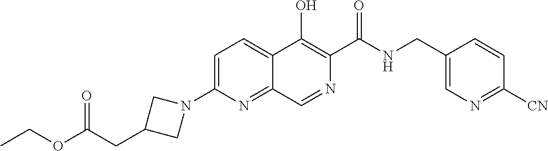 [SMILES: CCOC(=O)CC1CN(c2ccc3c(O)c(C(=O)NCc4ccc(C#N)nc4)ncc3n2)C1] |
| 154 | 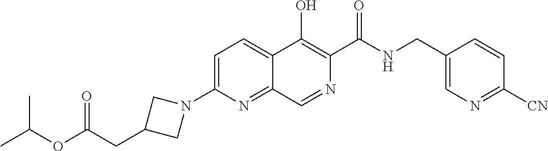 [SMILES: CC(C)OC(=O)CC1CN(c2ccc3c(O)c(C(=O)NCc4ccc(C#N)nc4)ncc3n2)C1] |
| 155 | 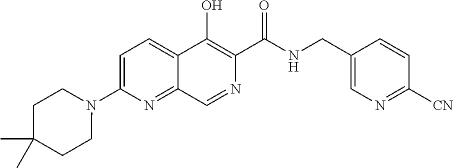 [SMILES: CC1(C)CCN(c2ccc3c(O)c(C(=O)NCc4ccc(C#N)nc4)ncc3n2)CC1] |
| 156 | 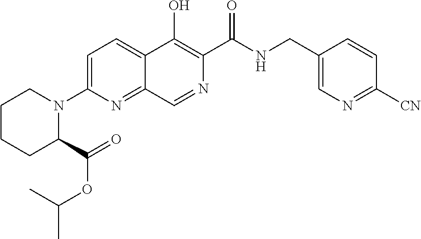 [SMILES: CC(C)OC(=O)[C@H]1CCCCN1c1ccc2c(O)c(C(=O)NCc3ccc(C#N)nc3)ncc2n1] |
| 157 | 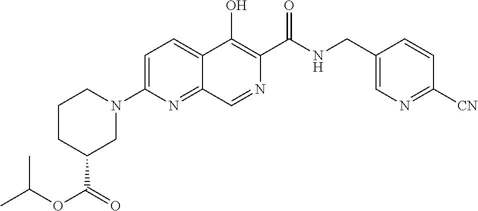 [SMILES: CC(C)OC(=O)[C@@H]1CCCN(c2ccc3c(O)c(C(=O)NCc4ccc(C#N)nc4)ncc3n2)C1] |
| 158 | 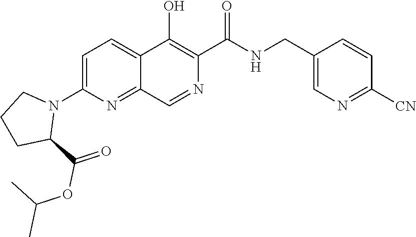 [SMILES: CC(C)OC(=O)[C@H]1CCCN1c1ccc2c(O)c(C(=O)NCc3ccc(C#N)nc3)ncc2n1] |
| 160 | 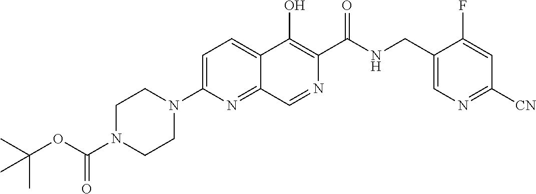 [SMILES: CC(C)(C)OC(=O)N1CCN(c2ccc3c(O)c(C(=O)NCc4cnc(C#N)cc4F)ncc3n2)CC1] |
| 163 | 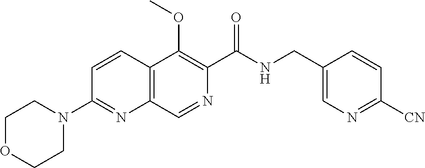 [SMILES: COc1c(C(=O)NCc2ccc(C#N)nc2)ncc2nc(N3CCOCC3)ccc12] |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, tranls, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H (T), ¹³C, ¹⁴C, ¹⁵N, ¹⁷O, ¹⁷O, ³¹P, ³²P, ³⁵S, ¹⁸F, and ³⁶Cl, respectively. Compounds described herein, and the pharmaceutically acceptable salts, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Combinations

Described herein are methods of treating an inflammatory epithelial disease in a subject in need thereof, the method comprising the administering to the subject a compound disclosed herein and an additional agent or a pharmaceutically acceptable salt thereof.

Described herein are methods of treating an inflammatory epithelial disease in a subject in need thereof, the method comprising the administering to the subject: a compound of Formula (I), or a pharmaceutically acceptable salt, or stereoisomer thereof:

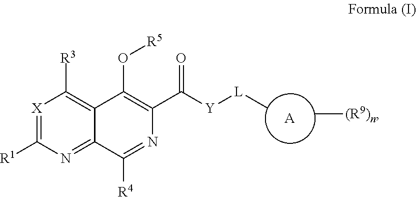

wherein:
R¹ monocyclic heterocycloalkyl optionally and independently substituted with one or more R^{1a};
each R^{1a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R⁸, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR¹S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{1a} on the same atom are taken together to form an oxo;
X is N or CR²;
R² is hydrogen, fluoro, chloro, bromo, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R³ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁴ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁵ is hydrogen, C₁-C₆alkyl, C₁-C₆haloalkyl, C₁-C₆hydroxyalkyl, C₁-C₆aminoalkyl, or C₁-C₆heteroalkyl;
Y is —O—, —S—, or —NR⁶—;
R⁶ is hydrogen, C₁-C₆alkyl, C₁-C₆haloalkyl, C₁-C₆hydroxyalkyl, C₁-C₆aminoalkyl, or C₁-C₆heteroalkyl;
L is —(CR⁷R⁸)ₚ—;
each R⁷ and R⁸ are independently hydrogen, C₁-C₆alkyl, C₁-C₆haloalkyl, C₁-C₆hydroxyalkyl, C₁-C₆aminoalkyl, or C₁-C₆heteroalkyl;
or R⁷ and R⁸ on the same carbon are taken together to form a cycloalkyl or heterocycloalkyl; each optionally substituted with one or more R^{7a};
each R^{7a} is independently halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
p is 0-4;
Ring A is cycloalkyl, heterocycloalkyl, aryl, or heteroaryl; each R⁹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{9a} on the same atom are taken together to form an oxo;
n is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆(alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; or
two R on the same atom are taken together to form an oxo; (b) an additional agent or a pharmaceutically acceptable salt thereof;
wherein the combined amount of (a) and (b) is therapeutically effective for the treating of the inflammatory epithelial disease.

Described herein are methods of treating an inflammatory epithelial disease in a subject in need thereof, the method comprising the administering to the subject: a compound of Table 1, or a pharmaceutically acceptable salt, or stereoisomer thereof and an additional agent or a pharmaceutically acceptable salt thereof.

Described herein are methods of treating an inflammatory epithelial disease in a subject in need thereof, the method comprising the administering to the subject: compound 29 or a pharmaceutically acceptable salt, or stereoisomer thereof and an additional agent or a pharmaceutically acceptable salt thereof.

### Additional Agents

In some embodiments, the additional agent is a 5-aminosalicylates (5-ASAs), anti-inflammatory agent, an ASK1 inhibitor, an alpha-fetoprotein modulator, an adenosine A3 receptor antagonist, an adrenomedullin ligand, an AKT1 gene inhibitor, an anti-CD28 inhibitor, an antibiotic, an antifungal, an ATPase inhibitor, a beta adrenoceptor antagonist, a BTK inhibitor, a beta-glucuronidase inhibitor, a bradykinin receptor modulator, a calcineurin inhibitor, a chaperonin binding immunoglobulin protein, a calcium channel inhibitor, a cathepsin S inhibitor, a CCR3 chemokine antagonist, a CD40 ligand receptor antagonist, a chemokine CXC ligand inhibitor, a CHST15 gene inhibitor, a collagen modulator, a CSF-1 antagonist, a cyclooxygenase inhibitor, a cytochrome P450 3A4 inhibitor, a carbohydrate metabolism modulator, a CCR9 chemokine antagonist, a CD233 modulator, a CD29 modulator, a CD3 antagonist, a CD4 antagonist, a CD40 ligand inhibitor, a CD40 gene inhibitor, a CX3CR1 chemokine modulator, a COT protein kinase inhibitor, an eotaxin ligand inhibitor, an EP4 prostanoid receptor agonist, an erythropoietin receptor agonist, an ecobiotic, an F1F0 ATP synthase modulator, a farnesoid X receptor (FXR and NR1H4) agonist or modulator, a fecal microbiota transplantation (FMT), a fractalkine ligand inhibitor, a free fatty acid receptor 2 antagonist, a GATA 3 transcription factor inhibitor, a glucagon-like peptide 2 agonist, a glucocorticoid receptor modulator, glucocorticoid receptor agonist, a guanylate cyclase receptor agonist, a histone deacetylase inhibitor, a histone deacetylase-6 inhibitor, an HLA class II antigen modulator, an immunosuppressant, an IL-12 antagonist, an IL-13 antagonist, an IL-23 antagonist, an IL-6 antagonist, an IL-6 receptor modulator, an IL-7 receptor modulator, an IL-7 antagonist, an IL-8 antagonist, an integrin alpha-4/beta-1 antagonist, an integrin alpha-4/beta-7 antagonist, an integrin alpha-E antagonist, an integrin antagonist, an integrin beta-7 antagonist, an interleukin ligand inhibitor, an interleukin-2 ligand, an interleukin receptor 17A antagonist, an interleukin-1 beta ligand, an interleukin-1 beta ligand modulator, an IRAK4 inhibitor, an ICAM1 gene inhibitor, an IL-1 beta ligand modulator, an IL-18 antagonist, an IL-22 agonist, an IL-23A inhibitor, an interleukin 1 like receptor 2 inhibitor, a JAK inhibitor, a JAK tyrosine kinase inhibitor, a Jak1 tyrosine kinase inhibitor, a Jak3 tyrosine kinase inhibitor, an LanC like protein 2 modulator, a lipoxygenase modulator, a lactoferrin stimulator, a leukocyte elastate inhibitor, a leukocyte proteinase-3 inhibitor, a MAdCAM inhibitor, a matrix metalloprotease inhibitor, a melanocortin agonist, a melanocortin MC1 agonist, a metalloprotease-9 inhibitor, a melanin concentrating hormone (MCH-1) antagonist, a microbiome modulator, a natriuretic peptide receptor C agonist, a neuregulin-4 ligand, an NKG2 D activating NK receptor antagonist, an NLRP3 inhibitor, a neuregulin-4 ligand, a nuclear factor kappa B inhibitor, an opioid receptor antagonist, an opioid receptor delta antagonist, an oxidoreductase inhibitor, an OX40 ligand inhibitor, a Pellino homolog 1 inhibitor, a P2X7 purinoceptor agonist, a PDE 4 inhibitor, a phagocytosis stimulating peptide modulator, a potassium channel inhibitor, a PPAR alpha agonist, a PPAR delta agonist, a PPAR gamma agonist, a protein fimH inhibitor, a P-selectin glycoprotein ligand-1 inhibitor, a Ret tyrosine kinase receptor inhibitor, an RNA polymerase inhibitor, a RIP-1 kinase inhibitor, a RIP-2 kinase inhibitor, a tissue transglutaminase inhibitor, a sphingosine-1-phosphate phosphatase-1 stimulator, a sphingosine-1-phosphate phosphatase modulator, a sphingosine-1-phosphate receptor-1 agonist, a sphingosine-1-phosphate receptor-1 antagonist, a sphingosine-1-phosphate receptor-1 modulator, a sphingosine-1-phosphate receptor-5 modulator, a STAT3 gene inhibitor, a stem cell antigen-1 inhibitor, a superoxide dismutase modulator, a superoxide dismutase stimulator, an SYK inhibitor, a TGF beta 1 ligand inhibitor, a thymulin agonist, a TLR antagonist, a TLR agonist, a TNF alpha ligand inhibitor, a TNF antagonist, a tumor necrosis factor superfamily member 14 modulator, a type II TNF receptor modulator, a Tpl 2 inhibitor, a TLR-3 antagonist, a TLR-4 antagonist, a TLR8 inhibitor, a TLR-9 agonist, a TNF ligand inhibitor, a TNF alpha ligand modulator, a TPL-2 inhibitor, a tumor necrosis factor superfamily member 15 inhibitor, a Tyk2 tyrosine kinase inhibitor, a type I IL-1 receptor antagonist, a TrkA receptor antagonist, a vanilloid VR1 agonist, a zonulin inhibitor, or a pharmaceutically acceptable salt thereof.

In some embodiments, the additional agent is a 5-aminosalicylates (5-ASAs) or a pharmaceutically acceptable salt thereof. In some embodiments, the additional agent is anti-inflammatory agent or a pharmaceutically acceptable salt thereof. In some embodiments, the additional agent is an ASK1 inhibitor or a pharmaceutically acceptable salt thereof. In some embodiments, the additional agent is an alpha-fetoprotein modulator or a pharmaceutically acceptable salt thereof. In some embodiments, the additional agent is an adenosine A3 receptor antagonist or a pharmaceutically acceptable salt thereof. In some embodiments, the additional agent is an adrenomedullin ligand or a pharmaceutically acceptable salt thereof. In some embodiments, the additional agent is an AKT1 gene inhibitor or a pharmaceutically acceptable salt thereof. In some embodiments, the additional agent is an anti-CD28 inhibitor or a pharmaceutically acceptable salt thereof. In some embodiments, the additional agent is an antibiotic or a pharmaceutically acceptable salt thereof. In some embodiments, the additional agent is an antifungal or a pharmaceutically acceptable salt thereof. In some embodiments, the additional agent is an ATPase inhibitor or a pharmaceutically acceptable salt thereof. In some embodiments, the additional agent is a beta adrenoceptor antagonist or a pharmaceutically acceptable salt thereof. In some embodiments, the additional agent is a BTK inhibitor or a pharmaceutically acceptable salt thereof. In some embodiments, the additional agent is a beta-glucuronidase inhibitor or a pharmaceutically acceptable salt thereof. In some embodiments, the additional agent is a bradykinin receptor modulator or a pharmaceutically acceptable salt thereof. In some embodiments, the additional agent is a calcineurin inhibitor or a pharmaceutically acceptable salt thereof. In some embodiments, the additional agent is a chaperonin binding immunoglobulin protein or a pharmaceutically acceptable salt thereof. In some embodiments, the additional agent is a calcium channel inhibitor or a pharmaceutically acceptable salt thereof. In some embodiments, the additional agent is a cathepsin S inhibitor or a pharmaceutically acceptable salt thereof. In some embodiments, the additional agent is a CCR3 chemokine antagonist or a pharmaceutically acceptable salt thereof. In some embodiments, the additional agent is a CD40 ligand receptor antagonist or a pharmaceutically acceptable salt thereof. In some embodiments, the additional agent is a chemokine CXC ligand inhibitor or a pharmaceutically acceptable salt thereof. In some embodiments, the additional agent is a CHST15 gene inhibitor or a pharmaceutically acceptable salt thereof. In some embodiments, the additional agent is a collagen modulator or a pharmaceutically acceptable salt thereof. In some embodiments, the additional agent is a CSF-1 antagonist or a pharmaceutically acceptable salt thereof. In some embodiments, the additional agent is a cyclooxygenase inhibitor or a pharmaceutically acceptable salt thereof. In some embodiments, the additional agent is a cytochrome P450 3A4 inhibitor or a pharmaceutically acceptable salt thereof. In some embodiments, the additional agent is a carbohydrate metabolism modulator or a pharmaceutically acceptable salt thereof. In some embodiments, the additional agent is a CCR9 chemokine antagonist or a pharmaceutically acceptable salt thereof. In some embodiments, the additional agent is a CD233 modulator or a pharmaceutically acceptable salt thereof. In some embodiments, the additional agent is a CD29 modulator or a pharmaceutically acceptable salt thereof. In some embodiments, the additional agent is a CD3 antagonist or a pharmaceutically acceptable salt thereof. In some embodiments, the additional agent is a CD4 antagonist or a pharmaceutically acceptable salt thereof. In some embodiments, the additional agent is a CD40 ligand inhibitor or a pharmaceutically acceptable salt thereof. In some embodiments, the additional agent is a CD40 gene inhibitor or a pharmaceutically acceptable salt thereof. In some embodiments, the additional agent is a CX3CR1 chemokine modulator or a pharmaceutically acceptable salt thereof. In some embodiments, the additional agent is a COT protein kinase inhibitor or a pharmaceutically acceptable salt thereof. In some embodiments, the additional agent is an eotaxin ligand inhibitor or a pharmaceutically acceptable salt thereof. In some embodiments, the additional agent is an EP4 prostanoid receptor agonist or a pharmaceutically acceptable salt thereof. In some embodiments, the additional agent is an erythropoietin receptor agonist or a pharmaceutically acceptable salt thereof. In some embodiments, the additional agent is an ecobiotic or a pharmaceutically acceptable salt thereof. In some embodiments, the additional agent is an F1F0 ATP synthase modulator or a pharmaceutically acceptable salt thereof. In some embodiments, the additional agent is a farnesoid X receptor (FXR and NR1H4) agonist or modulator or a pharmaceutically acceptable salt thereof. In some embodiments, the additional agent is a fecal microbiota transplantation (FMT) or a pharmaceutically acceptable salt thereof. In some embodiments, the additional agent is a fractalkine ligand inhibitor or a pharmaceutically acceptable salt thereof. In some embodiments, the additional agent is a free fatty acid receptor 2 antagonist or a pharmaceutically acceptable salt thereof. In some embodiments, the additional agent is a GATA 3 transcription factor inhibitor or a pharmaceutically acceptable salt thereof. In some embodiments, the additional agent is a glucagon-like peptide 2 agonist or a pharmaceutically acceptable salt thereof. In some embodiments, the additional agent is a glucocorticoid receptor modulator or a pharmaceutically acceptable salt thereof. In some embodiments, the additional agent is glucocorticoid receptor agonist or a pharmaceutically acceptable salt thereof. In some embodiments, the additional agent is a guanylate cyclase receptor agonist or a pharmaceutically acceptable salt thereof. In some embodiments, the additional agent is a histone deacetylase inhibitor or a pharmaceutically acceptable salt thereof. In some embodiments, the additional agent is a histone deacetylase-6 inhibitor or a pharmaceutically acceptable salt thereof. In some embodiments, the additional agent is an HLA class II antigen modulator or a pharmaceutically acceptable salt thereof. In some embodiments, the additional agent is an immunosuppressant or a pharmaceutically acceptable salt thereof. In some embodiments, the additional agent is an IL-12 antagonist or a pharmaceutically acceptable salt thereof. In some embodiments, the additional agent is an IL-13 antagonist or a pharmaceutically acceptable salt thereof. In some embodiments, the additional agent is an IL-23 antagonist or a pharmaceutically acceptable salt thereof. In some embodiments, the additional agent is an IL-6 antagonist or a pharmaceutically acceptable salt thereof. In some embodiments, the additional agent is an IL-6 receptor modulator or a pharmaceutically acceptable salt thereof. In some embodiments, the additional agent is an IL-7 receptor modulator or a pharmaceutically acceptable salt thereof. In some embodiments, the additional agent is an IL-7 antagonist or a pharmaceutically acceptable salt thereof. In some embodiments, the additional agent is an IL-8 antagonist or a pharmaceutically acceptable salt thereof. In some embodiments, the additional agent is an integrin alpha-4/beta-1 antagonist or a pharmaceutically acceptable salt thereof. In some embodiments, the additional agent is an integrin alpha-4/beta-7 antagonist or a pharmaceutically acceptable salt thereof. In some embodiments, the additional agent is an integrin alpha-E antagonist or a pharmaceutically acceptable salt thereof. In some embodiments, the additional agent is an integrin antagonist or a pharmaceutically acceptable salt thereof. In some embodiments, the additional agent is an integrin beta-7 antagonist or a pharmaceutically acceptable salt thereof. In some embodiments, the additional agent is an interleukin ligand inhibitor or a pharmaceutically acceptable salt thereof. In some embodiments, the additional agent is an interleukin-2 ligand or a pharmaceutically acceptable salt thereof. In some embodiments, the additional agent is an interleukin receptor 17A antagonist or a pharmaceutically acceptable salt thereof. In some embodiments, the additional agent is an interleukin-1 beta ligand or a pharmaceutically acceptable salt thereof. In some embodiments, the additional agent is an interleukin-1 beta ligand modulator or a pharmaceutically acceptable salt thereof. In some embodiments, the additional agent is an IRAK4 inhibitor or a pharmaceutically acceptable salt thereof. In some embodiments, the additional agent is an ICAM1 gene inhibitor or a pharmaceutically acceptable salt thereof. In some embodiments, the additional agent is an IL-1 beta ligand modulator or a pharmaceutically acceptable salt thereof. In some embodiments, the additional agent is an IL-18 antagonist or a pharmaceutically acceptable salt thereof. In some embodiments, the additional agent is an IL-22 agonist or a pharmaceutically acceptable salt thereof. In some embodiments, the additional agent is an IL-23A inhibitor or a pharmaceutically acceptable salt thereof. In some embodiments, the additional agent is an interleukin 1 like receptor 2 inhibitor or a pharmaceutically acceptable salt thereof. In some embodiments, the additional agent is a JAK inhibitor or a pharmaceutically acceptable salt thereof. In some embodiments, the additional agent is a JAK tyrosine kinase inhibitor or a pharmaceutically acceptable salt thereof. In some embodiments, the additional agent is a Jak1 tyrosine kinase inhibitor. In some embodiments, the additional agent is a Jak3 tyrosine kinase inhibitor or a pharmaceutically acceptable salt thereof. In some embodiments, the additional agent is an LanC like protein 2 modulator or a pharmaceutically acceptable salt thereof. In some embodiments, the additional agent is a lipoxygenase modulator or a pharmaceutically acceptable salt thereof. In some embodiments, the additional agent is a lactoferrin stimulator or a pharmaceutically acceptable salt thereof. In some embodiments, the additional agent is a leukocyte elastate inhibitor or a pharmaceutically acceptable salt thereof. In some embodiments, the additional agent is a leukocyte proteinase-3 inhibitor or a pharmaceutically acceptable salt thereof. In some embodiments, the additional agent is a MAdCAM inhibitor or a pharmaceutically acceptable salt thereof. In some embodiments, the additional agent is a matrix metalloprotease inhibitor or a pharmaceutically acceptable salt thereof. In some embodiments, the additional agent is a melanocortin agonist or a pharmaceutically acceptable salt thereof. In some embodiments, the additional agent is a melanocortin MC1 agonist or a pharmaceutically acceptable salt thereof. In some embodiments, the additional agent is a metalloprotease-9 inhibitor or a pharmaceutically acceptable salt thereof. In some embodiments, the additional agent is a melanin concentrating hormone (MCH-1) antagonist or a pharmaceutically acceptable salt thereof. In some embodiments, the additional agent is a microbiome modulator or a pharmaceutically acceptable salt thereof. In some embodiments, the additional agent is a natriuretic peptide receptor C agonist or a pharmaceutically acceptable salt thereof. In some embodiments, the additional agent is a neuregulin-4 ligand or a pharmaceutically acceptable salt thereof. In some embodiments, the additional agent is an NKG2 D activating NK receptor antagonist or a pharmaceutically acceptable salt thereof. In some embodiments, the additional agent is an NLRP3 inhibitor or a pharmaceutically acceptable salt thereof. In some embodiments, the additional agent is a neuregulin-4 ligand or a pharmaceutically acceptable salt thereof. In some embodiments, the additional agent is a nuclear factor kappa B inhibitor or a pharmaceutically acceptable salt thereof. In some embodiments, the additional agent is an opioid receptor antagonist or a pharmaceutically acceptable salt thereof. In some embodiments, the additional agent is an opioid receptor delta antagonist or a pharmaceutically acceptable salt thereof. In some embodiments, the additional agent is an oxidoreductase inhibitor or a pharmaceutically acceptable salt thereof. In some embodiments, the additional agent is an OX40 ligand inhibitor or a pharmaceutically acceptable salt thereof. In some embodiments, the additional agent is a Pellino homolog 1 inhibitor or a pharmaceutically acceptable salt thereof. In some embodiments, the additional agent is a P2X7 purinoceptor agonist or a pharmaceutically acceptable salt thereof. In some embodiments, the additional agent is a PDE 4 inhibitor or a pharmaceutically acceptable salt thereof. In some embodiments, the additional agent is a phagocytosis stimulating peptide modulator or a pharmaceutically acceptable salt thereof. In some embodiments, the additional agent is a potassium channel inhibitor or a pharmaceutically acceptable salt thereof. In some embodiments, the additional agent is a PPAR alpha agonist or a pharmaceutically acceptable salt thereof. In some embodiments, the additional agent is a PPAR delta agonist or a pharmaceutically acceptable salt thereof. In some embodiments, the additional agent is a PPAR gamma agonist or a pharmaceutically acceptable salt thereof. In some embodiments, the additional agent is a protein fimH inhibitor or a pharmaceutically acceptable salt thereof. In some embodiments, the additional agent is a P-selectin glycoprotein ligand-1 inhibitor or a pharmaceutically acceptable salt thereof. In some embodiments, the additional agent is a Ret tyrosine kinase receptor inhibitor or a pharmaceutically acceptable salt thereof. In some embodiments, the additional agent is an RNA polymerase inhibitor or a pharmaceutically acceptable salt thereof. In some embodiments, the additional agent is a RIP-1 kinase inhibitor or a pharmaceutically acceptable salt thereof. In some embodiments, the additional agent is a RIP-2 kinase inhibitor or a pharmaceutically acceptable salt thereof. In some embodiments, the additional agent is a tissue transglutaminase inhibitor or a pharmaceutically acceptable salt thereof. In some embodiments, the additional agent is a sphingosine-1-phosphate phosphatase-1 stimulator or a pharmaceutically acceptable salt thereof. In some embodiments, the additional agent is a sphingosine-1-phosphate phosphatase modulator or a pharmaceutically acceptable salt thereof. In some embodiments, the additional agent is a sphingosine-1-phosphate receptor-1 agonist or a pharmaceutically acceptable salt thereof. In some embodiments, the additional agent is a sphingosine-1-phosphate receptor-1 antagonist or a pharmaceutically acceptable salt thereof. In some embodiments, the additional agent is a sphingosine-1-phosphate receptor-1 modulator or a pharmaceutically acceptable salt thereof. In some embodiments, the additional agent is a sphingosine-1-phosphate receptor-5 modulator or a pharmaceutically acceptable salt thereof. In some embodiments, the additional agent is a STAT3 gene inhibitor or a pharmaceutically acceptable salt thereof. In some embodiments, the additional agent is a stem cell antigen-1 inhibitor or a pharmaceutically acceptable salt thereof. In some embodiments, the additional agent is a superoxide dismutase modulator or a pharmaceutically acceptable salt thereof. In some embodiments, the additional agent is a superoxide dismutase stimulator or a pharmaceutically acceptable salt thereof. In some embodiments, the additional agent is an SYK inhibitor or a pharmaceutically acceptable salt thereof. In some embodiments, the additional agent is a TGF beta 1 ligand inhibitor or a pharmaceutically acceptable salt thereof. In some embodiments, the additional agent is a thymulin agonist or a pharmaceutically acceptable salt thereof. In some embodiments, the additional agent is a TLR antagonist or a pharmaceutically acceptable salt thereof. In some embodiments, the additional agent is a TLR agonist or a pharmaceutically acceptable salt thereof. In some embodiments, the additional agent is a TNF alpha ligand inhibitor or a pharmaceutically acceptable salt thereof. In some embodiments, the additional agent is a TNF antagonist or a pharmaceutically acceptable salt thereof. In some embodiments, the additional agent is a tumor necrosis factor superfamily member 14 modulator or a pharmaceutically acceptable salt thereof. In some embodiments, the additional agent is a type II TNF receptor modulator or a pharmaceutically acceptable salt thereof. In some embodiments, the additional agent is a Tpl 2 inhibitor or a pharmaceutically acceptable salt thereof. In some embodiments, the additional agent is a TLR-3 antagonist or a pharmaceutically acceptable salt thereof. In some embodiments, the additional agent is a TLR-4 antagonist or a pharmaceutically acceptable salt thereof. In some embodiments, the additional agent is a TLR8 inhibitor or a pharmaceutically acceptable salt thereof. In some embodiments, the additional agent is a TLR-9 agonist or a pharmaceutically acceptable salt thereof. In some embodiments, the additional agent is a TNF ligand inhibitor. In some embodiments, the additional agent is a TNF alpha ligand modulator or a pharmaceutically acceptable salt thereof. In some embodiments, the additional agent is a TPL-2 inhibitor or a pharmaceutically acceptable salt thereof. In some embodiments, the additional agent is a tumor necrosis factor superfamily member 15 inhibitor or a pharmaceutically acceptable salt thereof. In some embodiments, the additional agent is a Tyk2 tyrosine kinase inhibitor or a pharmaceutically acceptable salt thereof. In some embodiments, the additional agent is a type I IL-1 receptor antagonist or a pharmaceutically acceptable salt thereof. In some embodiments, the additional agent is a TrkA receptor antagonist or a pharmaceutically acceptable salt thereof. In some embodiments, the additional agent is a vanilloid VR1 agonist or a pharmaceutically acceptable salt thereof. In some embodiments, the additional agent is a zonulin inhibitor or a pharmaceutically acceptable salt thereof.

In some embodiments, the additional agent is a 5-ASAs or a pharmaceutically acceptable salt thereof. In some embodiments, the 5-ASAs is balsalazide, sulphasalazine, mesalazine, olsalazine, mesalamine, or a pharmaceutically acceptable salt thereof. In some embodiments, the 5-ASAs is balsalazide or a pharmaceutically acceptable salt thereof. In some embodiments, the 5-ASAs is sulphasalazine or a pharmaceutically acceptable salt thereof. In some embodiments, the 5-ASAs is mesalazine or a pharmaceutically acceptable salt thereof. In some embodiments, the 5-ASAs is olsalazine or a pharmaceutically acceptable salt thereof. In some embodiments, the 5-ASAs is mesalamine or a pharmaceutically acceptable salt thereof.

In some embodiments, the additional agent is an anti-inflammatory agent or a pharmaceutically acceptable salt thereof. In some embodiments, the anti-inflammatory agent is a corticosteroid, a non-steroidal anti-inflammatory drug (NSAID), non-specific cyclooxygenase enzyme inhibitor, or COX-2 specific cyclooxygenase enzyme inhibitor, or a pharmaceutically acceptable salt thereof. In some embodiments, the anti-inflammatory agent is a corticosteroid. In some embodiments, the corticosteroid is cortisone or a pharmaceutically acceptable salt thereof. In some embodiments, the corticosteroid is dexamethasone or a pharmaceutically acceptable salt thereof. In some embodiments, the corticosteroid is prednisolone or a pharmaceutically acceptable salt thereof. In some embodiments, the corticosteroid is prednisolone sodium phosphate. In some embodiments, the anti-inflammatory agent is a non-steroidal anti-inflammatory drug (NSAID) or a pharmaceutically acceptable salt thereof. In some embodiments, the NSAID is ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, hydroxychloroquine, or a pharmaceutically acceptable salt thereof. In some embodiments, the NSAID is ibuprofen or a pharmaceutically acceptable salt thereof. In some embodiments, the NSAID is flurbiprofen or a pharmaceutically acceptable salt thereof. In some embodiments, the NSAID is naproxen or a pharmaceutically acceptable salt thereof. In some embodiments, the NSAID is naproxen sodium. In some embodiments, the NSAID is diclofenac or a pharmaceutically acceptable salt thereof. In some embodiments, the NSAID is combinations of diclofenac sodium and misoprostol or a pharmaceutically acceptable salt thereof. In some embodiments, the NSAID is sulindac or a pharmaceutically acceptable salt thereof. In some embodiments, the NSAID is oxaprozin or a pharmaceutically acceptable salt thereof. In some embodiments, the NSAID is diflunisal or a pharmaceutically acceptable salt thereof. In some embodiments, the NSAID is piroxicam or a pharmaceutically acceptable salt thereof. In some embodiments, the NSAID is indomethacin or a pharmaceutically acceptable salt thereof. In some embodiments, the NSAID is etodolac or a pharmaceutically acceptable salt thereof. In some embodiments, the NSAID is fenoprofen calcium. In some embodiments, the NSAID is ketoprofen or a pharmaceutically acceptable salt thereof. In some embodiments, the NSAID is sodium nabumetone. In some embodiments, the NSAID is sulfasalazine or a pharmaceutically acceptable salt thereof. In some embodiments, the NSAID is tolmetin sodium. In some embodiments, the NSAID is hydroxychloroquine or a pharmaceutically acceptable salt thereof. In some embodiments, the anti-inflammatory agent is the COX-2 specific cyclooxygenase enzyme inhibitor or a pharmaceutically acceptable salt thereof. In some embodiments, the COX-2 specific cyclooxygenase enzyme inhibitor is celecoxib, valdecoxib, lumiracoxib, etoricoxib, rofecoxib, or a pharmaceutically acceptable salt thereof. In some embodiments, the COX-2 specific cyclooxygenase enzyme inhibitor is celecoxib or a pharmaceutically acceptable salt thereof. In some embodiments, the COX-2 specific cyclooxygenase enzyme inhibitor is valdecoxib or a pharmaceutically acceptable salt thereof. In some embodiments, the COX-2 specific cyclooxygenase enzyme inhibitor is lumiracoxib or a pharmaceutically acceptable salt thereof. In some embodiments, the COX-2 specific cyclooxygenase enzyme inhibitor is etoricoxib or a pharmaceutically acceptable salt thereof. In some embodiments, the COX-2 specific cyclooxygenase enzyme inhibitor is rofecoxib or a pharmaceutically acceptable salt thereof. In some embodiments, the anti-inflammatory agent is gold sodium thiomalate, auranofin, or a pharmaceutically acceptable salt thereof. In some embodiments, the anti-inflammatory agent is gold sodium thiomalate or a pharmaceutically acceptable salt thereof. In some embodiments, the anti-inflammatory agent is auranofin or a pharmaceutically acceptable salt thereof.

In some embodiments, the anti-inflammatory agent is the immunosuppressant or pharmaceutically acceptable salt thereof. In some embodiments, the immunosuppressant is mercaptopurine, methotrexate, leflunomide, cyclosporine, tacrolimus, azathioprine, mycophenolate mofetil, or a pharmaceutically acceptable salt thereof. In some embodiments, the immunosuppressant is mercaptopurine or a pharmaceutically acceptable salt thereof. In some embodiments, the immunosuppressant is methotrexate or a pharmaceutically acceptable salt thereof. In some embodiments, the immunosuppressant is leflunomide or a pharmaceutically acceptable salt thereof. In some embodiments, the immunosuppressant is cyclosporine or a pharmaceutically acceptable salt thereof. In some embodiments, the immunosuppressant is cyclosporine or a pharmaceutically acceptable salt thereof. In some embodiments, the immunosuppressant is tacrolimus or a pharmaceutically acceptable salt thereof. In some embodiments, the immunosuppressant is azathioprine or a pharmaceutically acceptable salt thereof. In some embodiments, the immunosuppressant is mycophenolate mofetil or a pharmaceutically acceptable salt thereof.

In some embodiments, the additional agent is an ASK1 inhibitor or pharmaceutically acceptable salt thereof. In some embodiments, the ASK1 inhibitor is GS-4997 or a pharmaceutically acceptable salt thereof.

In some embodiments, the additional agent is a BTK inhibitor or pharmaceutically acceptable salt thereof. In some embodiments, the BTK inhibitor is GS-4059 or a pharmaceutically acceptable salt thereof.

In some embodiments, the additional agent is a beta adrenoceptor antagonist or pharmaceutically acceptable salt thereof. In some embodiments, the beta adrenoceptor antagonist is NM-001 or a pharmaceutically acceptable salt thereof.

In some embodiments, the additional agent is a calcineurin inhibitor or pharmaceutically acceptable salt thereof. In some embodiments, the calcineurin inhibitor is tacrolimus, ciclosporin, or a pharmaceutically acceptable salt thereof. In some embodiments, the calcineurin inhibitor is tacrolimus or a pharmaceutically acceptable salt thereof. In some embodiments, the calcineurin inhibitor is ciclosporin or a pharmaceutically acceptable salt thereof.

In some embodiments, the additional agent is a CD40 ligand receptor antagonist or pharmaceutically acceptable salt thereof. In some embodiments, the CD40 ligand receptor antagonist is FFP-104, BI-655064, or a pharmaceutically acceptable salt thereof. In some embodiments, the CD40 ligand receptor antagonist is FFP-104 or a pharmaceutically acceptable salt thereof. In some embodiments, the CD40 ligand receptor antagonist is BI-655064 or a pharmaceutically acceptable salt thereof.

In some embodiments, the additional agent is a cathepsin S inhibitor or pharmaceutically acceptable salt thereof. In some embodiments, the cathepsin S inhibitor is VBY-129 or a pharmaceutically acceptable salt thereof.

In some embodiments, the additional agent is a chemokine CXC ligand inhibitor or pharmaceutically acceptable salt thereof. In some embodiments, the chemokine CXC ligand inhibitor is LY-3041658 or a pharmaceutically acceptable salt thereof.

In some embodiments, the additional agent is a CHST15 gene inhibitor or pharmaceutically acceptable salt thereof. In some embodiments, the CHST15 gene inhibitor is STNM-01 or a pharmaceutically acceptable salt thereof.

In some embodiments, the additional agent is a collagen modulator or pharmaceutically acceptable salt thereof. In some embodiments, the collagen modulator is ECCS-50 (DCCT-10) or a pharmaceutically acceptable salt thereof.

In some embodiments, the additional agent is a CSF-1 antagonist or pharmaceutically acceptable salt thereof. In some embodiments, the CSF-1 antagonist is JNJ-40346527 (PRV-6527), SNDX-6352, or a pharmaceutically acceptable salt thereof. In some embodiments, the CSF-1 antagonist is JNJ-40346527 (PRV-6527) or a pharmaceutically acceptable salt thereof. In some embodiments, the CSF-1 antagonist is SNDX-6352 or a pharmaceutically acceptable salt thereof.

In some embodiments, the additional agent is an eotaxin ligand inhibitor or pharmaceutically acceptable salt thereof. In some embodiments, the eotaxin ligand inhibitor is bertilimumab or a pharmaceutically acceptable salt thereof.

In some embodiments, the additional agent is an EP4 prostanoid receptor agonist or pharmaceutically acceptable salt thereof. In some embodiments, the EP4 prostanoid receptor agonist is KAG-308 or a pharmaceutically acceptable salt thereof.

In some embodiments, the additional agent is a free fatty acid receptor 2 antagonist or pharmaceutically acceptable salt thereof. In some embodiments, the free fatty acid receptor 2 antagonist is GLPG-0974 or a pharmaceutically acceptable salt thereof.

In some embodiments, the additional agent is a GATA 3 transcription factor inhibitor or pharmaceutically acceptable salt thereof. In some embodiments, the GATA 3 transcription factor inhibitor is SB-012 or a pharmaceutically acceptable salt thereof.

In some embodiments, the additional agent is a GATA 3 transcription factor inhibitor or pharmaceutically acceptable salt thereof. In some embodiments, the GATA 3 transcription factor inhibitor is SB-012 or a pharmaceutically acceptable salt thereof.

In some embodiments, the additional agent is a fractalkine ligand inhibitor or pharmaceutically acceptable salt thereof. In some embodiments, the fractalkine ligand inhibitor is quetmolimab (E-6011) or a pharmaceutically acceptable salt thereof.

In some embodiments, the additional agent is a glucagon-like peptide 2 agonist or pharmaceutically acceptable salt thereof. In some embodiments, the glucagon-like peptide 2 agonist is teduglutide, apraglutide, or a pharmaceutically acceptable salt thereof. In some embodiments, the glucagon-like peptide 2 agonist is teduglutide or a pharmaceutically acceptable salt thereof. In some embodiments, the glucagon-like peptide 2 agonist is apraglutide or a pharmaceutically acceptable salt thereof.

In some embodiments, the additional agent is a guanylate cyclase receptor agonist or pharmaceutically acceptable salt thereof. In some embodiments, the guanylate cyclase receptor agonist is dolcanatide or a pharmaceutically acceptable salt thereof.

In some embodiments, the additional agent is a histone deacetylase inhibitor or pharmaceutically acceptable salt thereof. In some embodiments, the histone deacetylase inhibitor is givinostat or a pharmaceutically acceptable salt thereof.

In some embodiments, the additional agent is an HLA class II antigen modulator or pharmaceutically acceptable salt thereof. In some embodiments, the HLA class II antigen modulator is HLA class II protein modulator or a pharmaceutically acceptable salt thereof.

In some embodiments, the additional agent is an IL-12 antagonist or pharmaceutically acceptable salt thereof. In some embodiments, the IL-12 antagonist is ustekinumab (IL12/IL23), MEDI2070, or a pharmaceutically acceptable salt thereof. In some embodiments, the IL-12 antagonist is ustekinumab (IL12/IL23) or a pharmaceutically acceptable salt thereof. In some embodiments, the IL-12 antagonist is MEDI2070 or a pharmaceutically acceptable salt thereof.

In some embodiments, the additional agent is an IL-13 antagonist or pharmaceutically acceptable salt thereof. In some embodiments, the IL-13 antagonist is tralokinumab or a pharmaceutically acceptable salt thereof.

In some embodiments, the additional agent is an IL-23 antagonist or pharmaceutically acceptable salt thereof. In some embodiments, the IL-23 antagonist is tildrakizumab, risankizumab (BI-655066), mirikizumab (LY-3074828), brazikumab (AMG-139), PTG-200, or a pharmaceutically acceptable salt thereof. In some embodiments, the IL-23 antagonist is tildrakizumab or a pharmaceutically acceptable salt thereof. In some embodiments, the IL-23 antagonist is risankizumab (BI-655066) or a pharmaceutically acceptable salt thereof. In some embodiments, the IL-23 antagonist is mirikizumab (LY-3074828) or a pharmaceutically acceptable salt thereof. In some embodiments, the IL-23 antagonist is brazikumab (AMG-139) or a pharmaceutically acceptable salt thereof. In some embodiments, the IL-23 antagonist is PTG-200 or a pharmaceutically acceptable salt thereof.

In some embodiments, the additional agent is an IL-6 antagonist or pharmaceutically acceptable salt thereof. In some embodiments, the IL-6 antagonist is olokizumab or a pharmaceutically acceptable salt thereof.

In some embodiments, the additional agent is an IL-6 receptor modulator or pharmaceutically acceptable salt thereof. In some embodiments, the IL-6 receptor modulator is olamkicept or a pharmaceutically acceptable salt thereof.

In some embodiments, the additional agent is an IL-7 receptor antagonist or pharmaceutically acceptable salt thereof. In some embodiments, the IL-7 receptor antagonist is OSE-127 or a pharmaceutically acceptable salt thereof.

In some embodiments, the additional agent is an IL-8 receptor antagonist or pharmaceutically acceptable salt thereof. In some embodiments, the IL-8 receptor antagonist is clotrimazole or a pharmaceutically acceptable salt thereof.

In some embodiments, the additional agent is an integrin alpha-4/beta-7 antagonist or pharmaceutically acceptable salt thereof. In some embodiments, the integrin alpha-4/beta-7 antagonist is etrolizumab (a4b7/aEb7), vedolizumab, carotegast methyl, TRK-170 (a4b7/a4b1), PN-10943, PTG-100, or a pharmaceutically acceptable salt thereof. In some embodiments, the integrin alpha-4/beta-7 antagonist is etrolizumab (a4b7/aEb7) or a pharmaceutically acceptable salt thereof. In some embodiments, the integrin alpha-4/beta-7 antagonist is vedolizumab or a pharmaceutically acceptable salt thereof. In some embodiments, the integrin alpha-4/beta-7 antagonist is carotegast methyl or a pharmaceutically acceptable salt thereof. In some embodiments, the integrin alpha-4/beta-7 antagonist is TRK-170 (a4b7/a4b1) or a pharmaceutically acceptable salt thereof. In some embodiments, the integrin alpha-4/beta-7 antagonist is PN-10943 or a pharmaceutically acceptable salt thereof. In some embodiments, the integrin alpha-4/beta-7 antagonist is PTG-100 or a pharmaceutically acceptable salt thereof.

In some embodiments, the additional agent is an integrin antagonist or pharmaceutically acceptable salt thereof. In some embodiments, the integrin antagonist is E-6007 or a pharmaceutically acceptable salt thereof.

In some embodiments, the additional agent is an interleukin ligand inhibitor or pharmaceutically acceptable salt thereof. In some embodiments, the interleukin ligand inhibitor is bimekizumab (IL-17A/IL-17F) or a pharmaceutically acceptable salt thereof.

In some embodiments, the additional agent is an interleukin receptor 17A antagonist or pharmaceutically acceptable salt thereof. In some embodiments, the interleukin receptor 17A antagonist is brodalumab or a pharmaceutically acceptable salt thereof.

In some embodiments, the additional agent is an interleukin-1 beta ligand or pharmaceutically acceptable salt thereof. In some embodiments, the interleukin-1 beta ligand is K(D)PT or a pharmaceutically acceptable salt thereof.

In some embodiments, the additional agent is a JAK tyrosine kinase inhibitor or pharmaceutically acceptable salt thereof. In some embodiments, the JAK tyrosine kinase inhibitor is tofacitinib, peficitinib, TD-3504, TD-1473, or a pharmaceutically acceptable salt thereof. In some embodiments, the JAK tyrosine kinase inhibitor is tofacitinib or a pharmaceutically acceptable salt thereof. In some embodiments, the JAK tyrosine kinase inhibitor is tofacitinib, peficitinib, TD-3504, TD-1473, or a pharmaceutically acceptable salt thereof.

In some embodiments, the additional agent is a Jak3 tyrosine kinase inhibitor or pharmaceutically acceptable salt thereof. In some embodiments, the Jak3 tyrosine kinase inhibitor is PF-06651600 or a pharmaceutically acceptable salt thereof.

In some embodiments, the additional agent is a LanC like protein 2 modulator or pharmaceutically acceptable salt thereof. In some embodiments, the LanC like protein 2 modulator is BT-11 or a pharmaceutically acceptable salt thereof.

In some embodiments, the additional agent is a MAdCAM inhibitor or pharmaceutically acceptable salt thereof. In some embodiments, the MAdCAM inhibitor is SHP-647 (PF-547659) or a pharmaceutically acceptable salt thereof.

In some embodiments, the additional agent is a melanocortin MC1 receptor agonist or pharmaceutically acceptable salt thereof. In some embodiments, the melanocortin MC1 receptor agonist is ASP-3291, PL-8177, or a pharmaceutically acceptable salt thereof. In some embodiments, the melanocortin MC1 receptor agonist is ASP-3291 or a pharmaceutically acceptable salt thereof. In some embodiments, the melanocortin MC1 receptor agonist is PL-8177 or a pharmaceutically acceptable salt thereof.

In some embodiments, the additional agent is a natriuretic peptide receptor C agonist or pharmaceutically acceptable salt thereof. In some embodiments, the natriuretic peptide receptor C agonist is plecanatide or a pharmaceutically acceptable salt thereof.

In some embodiments, the additional agent is an opioid receptor antagonist or pharmaceutically acceptable salt thereof. In some embodiments, the opioid receptor antagonist is naltrexone, IRT-103, or a pharmaceutically acceptable salt thereof. In some embodiments, the opioid receptor antagonist is naltrexone or a pharmaceutically acceptable salt thereof. In some embodiments, the opioid receptor antagonist is IRT-103 or a pharmaceutically acceptable salt thereof.

In some embodiments, the additional agent is an oxidoreductase inhibitor or pharmaceutically acceptable salt thereof. In some embodiments, the oxidoreductase inhibitor is olsalazine or a pharmaceutically acceptable salt thereof.

In some embodiments, the additional agent is a P2X7 purinoceptor modulator or pharmaceutically acceptable salt thereof. In some embodiments, the P2X7 purinoceptor modulator is SGM-1019 or a pharmaceutically acceptable salt thereof.

In some embodiments, the additional agent is a PDE 4 inhibitor or pharmaceutically acceptable salt thereof. In some embodiments, the PDE 4 inhibitor is apremilast or a pharmaceutically acceptable salt thereof.

In some embodiments, the additional agent is a PPAR alpha agonist or a PPAR delta agonist or pharmaceutically acceptable salt thereof. In some embodiments, the PPAR alpha agonist or the PPAR delta agonist is elafibranor (GFT-1007) or a pharmaceutically acceptable salt thereof.

In some embodiments, the additional agent is a PPAR gamma agonist or pharmaceutically acceptable salt thereof. In some embodiments, the PPAR gamma agonist is GED-0507-34-Levo or a pharmaceutically acceptable salt thereof.

In some embodiments, the additional agent is a protein fimH inhibitor or pharmaceutically acceptable salt thereof. In some embodiments, the protein fimH inhibitor is sibofimloc (EB-8018) or a pharmaceutically acceptable salt thereof.

In some embodiments, the additional agent is a P-selectin glycoprotein ligand-1 inhibitor or pharmaceutically acceptable salt thereof. In some embodiments, the P-selectin glycoprotein ligand-1 inhibitor is SEL-K2, AbGn-168H, neihulizumab, or a pharmaceutically acceptable salt thereof. In some embodiments, the P-selectin glycoprotein ligand-1 inhibitor is SEL-K2 or a pharmaceutically acceptable salt thereof. In some embodiments, the P-selectin glycoprotein ligand-1 inhibitor is AbGn-168H or a pharmaceutically acceptable salt thereof. In some embodiments, the P-selectin glycoprotein ligand-1 inhibitor is neihulizumab or a pharmaceutically acceptable salt thereof.

In some embodiments, the additional agent is a sphingosine-1-phosphate phosphatase 1 stimulator or pharmaceutically acceptable salt thereof. In some embodiments, the sphingosine-1-phosphate phosphatase 1 stimulator is etrasimod or a pharmaceutically acceptable salt thereof.

In some embodiments, the additional agent is a sphingosine-1-phosphate receptor-1 agonist or pharmaceutically acceptable salt thereof. In some embodiments, the sphingosine-1-phosphate receptor-1 agonist is ozanimod, mocravimod (KRP-203), BMS-986166, or a pharmaceutically acceptable salt thereof. In some embodiments, the sphingosine-1-phosphate receptor-1 agonist is ozanimod or a pharmaceutically acceptable salt thereof. In some embodiments, the sphingosine-1-phosphate receptor-1 agonist is mocravimod (KRP-203) or a pharmaceutically acceptable salt thereof. In some embodiments, the sphingosine-1-phosphate receptor-1 agonist is BMS-986166 or a pharmaceutically acceptable salt thereof.

In some embodiments, the additional agent is a sphingosine-1-phosphate receptor-5 agonist or pharmaceutically acceptable salt thereof. In some embodiments, the sphingosine-1-phosphate receptor-5 agonist is ozanimod or a pharmaceutically acceptable salt thereof.

In some embodiments, the additional agent is a sphingosine-1-phosphate receptor-1 antagonist or pharmaceutically acceptable salt thereof. In some embodiments, the sphingosine-1-phosphate receptor-1 antagonist is amiselimod (MT-1303) or a pharmaceutically acceptable salt thereof.

In some embodiments, the additional agent is a sphingosine-1-phosphate receptor-1 modulator or pharmaceutically acceptable salt thereof. In some embodiments, sphingosine-1-phosphate receptor-1 modulator is OPL-002 or a pharmaceutically acceptable salt thereof.

In some embodiments, the additional agent is a stem cell antigen-1 inhibitor or pharmaceutically acceptable salt thereof. In some embodiments, the stem cell antigen-1 inhibitor is ampion (DMI-9523) or a pharmaceutically acceptable salt thereof.

In some embodiments, the additional agent is a superoxide dismutase modulator or pharmaceutically acceptable salt thereof. In some embodiments, the superoxide dismutase modulator is midismase or a pharmaceutically acceptable salt thereof.

In some embodiments, the additional agent is a SYK inhibitor or pharmaceutically acceptable salt thereof. In some embodiments, the SYK inhibitor is GS-9876 or a pharmaceutically acceptable salt thereof.

In some embodiments, the additional agent is a TNF alpha ligand inhibitor or pharmaceutically acceptable salt thereof. In some embodiments, the TNF alpha ligand inhibitor is adalimumab, certolizumab pegol, infliximab, golimumab, DLX-105, Debio-0512, HMPL-004, CYT-020-TNFQb, Hemay-007, V-565, anti-TNFα antibody, or a pharmaceutically acceptable salt thereof. In some embodiments, the TNF alpha ligand inhibitor is adalimumab or a pharmaceutically acceptable salt thereof. In some embodiments, the TNF alpha ligand inhibitor is certolizumab pegol or a pharmaceutically acceptable salt thereof. In some embodiments, the TNF alpha ligand inhibitor is infliximab or a pharmaceutically acceptable salt thereof. In some embodiments, the TNF alpha ligand inhibitor is golimumab or a pharmaceutically acceptable salt thereof. In some embodiments, the TNF alpha ligand inhibitor is DLX-105 or a pharmaceutically acceptable salt thereof. In some embodiments, the TNF alpha ligand inhibitor is Debio-0512 or a pharmaceutically acceptable salt thereof. In some embodiments, the TNF alpha ligand inhibitor is HMPL-004 or a pharmaceutically acceptable salt thereof. In some embodiments, the TNF alpha ligand inhibitor is CYT-020-TNFQb or a pharmaceutically acceptable salt thereof. In some embodiments, the TNF alpha ligand inhibitor is Hemay-007 or a pharmaceutically acceptable salt thereof. In some embodiments, the TNF alpha ligand inhibitor is V-565 or a pharmaceutically acceptable salt thereof. In some embodiments, the TNF alpha ligand inhibitor is anti-TNFα antibody or a pharmaceutically acceptable salt thereof.

In some embodiments, the additional agent is a tumor necrosis factor superfamily member 14 modulator or pharmaceutically acceptable salt thereof. In some embodiments, the tumor necrosis factor superfamily member 14 modulator is AEVI-002 or a pharmaceutically acceptable salt thereof.

In some embodiments, the additional agent is a TNF antagonist or pharmaceutically acceptable salt thereof. In some embodiments, the TNF antagonist is AVX-470, tulinercept, etanercept, or a pharmaceutically acceptable salt thereof. In some embodiments, the TNF antagonist is AVX-470 or a pharmaceutically acceptable salt thereof. In some embodiments, the TNF antagonist is tulinercept or a pharmaceutically acceptable salt thereof. In some embodiments, the TNF antagonist is etanercept or a pharmaceutically acceptable salt thereof.

In some embodiments, the additional agent is a zonulin inhibitor or pharmaceutically acceptable salt thereof. In some embodiments, the zonulin inhibitor is larazotide acetate or a pharmaceutically acceptable salt thereof.

In some embodiments, the additional agent is a CD40 ligand inhibitor or pharmaceutically acceptable salt thereof. In some embodiments, the CD40 ligand inhibitor is SAR-441344, letolizumab, or a pharmaceutically acceptable salt thereof. In some embodiments, the CD40 ligand inhibitor is SAR-441344 or a pharmaceutically acceptable salt thereof. In some embodiments, the CD40 ligand inhibitor is letolizumab or a pharmaceutically acceptable salt thereof.

In some embodiments, the additional agent is an adenosine A3 receptor antagonist or pharmaceutically acceptable salt thereof. In some embodiments, the adenosine A3 receptor antagonist is PBF-677 or a pharmaceutically acceptable salt thereof.

In some embodiments, the additional agent is an adrenomedullin ligand or pharmaceutically acceptable salt thereof. In some embodiments, the adrenomedullin ligand is adrenomedullin or a pharmaceutically acceptable salt thereof.

In some embodiments, the additional agent is an antibiotic or pharmaceutically acceptable salt thereof. In some embodiments, the antibiotic is ciprofloxacin, clarithromycin, metronidazole, vancomycin, rifamycin, rifaximin, tosufloxacin, or a pharmaceutically acceptable salt thereof. In some embodiments, the antibiotic is ciprofloxacin or a pharmaceutically acceptable salt thereof. In some embodiments, the antibiotic is clarithromycin or a pharmaceutically acceptable salt thereof. In some embodiments, the antibiotic is metronidazole or a pharmaceutically acceptable salt thereof. In some embodiments, the antibiotic is vancomycin or a pharmaceutically acceptable salt thereof. In some embodiments, the antibiotic is rifamycin or a pharmaceutically acceptable salt thereof. In some embodiments, the antibiotic is rifaximin or a pharmaceutically acceptable salt thereof. In some embodiments, the antibiotic is tosufloxacin or a pharmaceutically acceptable salt thereof.

In some embodiments, the additional agent is an alpha-fetoprotein modulator or pharmaceutically acceptable salt thereof. In some embodiments, the alpha-fetoprotein modulator is ACT-101 or a pharmaceutically acceptable salt thereof.

In some embodiments, the additional agent is a carbohydrate metabolism modulator or pharmaceutically acceptable salt thereof. In some embodiments, the carbohydrate metabolism modulator is ASD-003 or a pharmaceutically acceptable salt thereof.

In some embodiments, the additional agent is a CCR9 chemokine antagonist or pharmaceutically acceptable salt thereof. In some embodiments, the CCR9 chemokine antagonist is CCX-507 or a pharmaceutically acceptable salt thereof.

In some embodiments, the additional agent is a CD233 modulator or pharmaceutically acceptable salt thereof. In some embodiments, the CD233 modulator is GSK-2831781 or a pharmaceutically acceptable salt thereof.

In some embodiments, the additional agent is a CD29 modulator or pharmaceutically acceptable salt thereof. In some embodiments, the CD29 modulator is PF-06687234 or a pharmaceutically acceptable salt thereof.

In some embodiments, the additional agent is a CX3CR1 chemokine modulator or pharmaceutically acceptable salt thereof. In some embodiments, the CX3CR1 chemokine modulator is E-6130 or a pharmaceutically acceptable salt thereof.

In some embodiments, the additional agent is an ecobiotic or pharmaceutically acceptable salt thereof. In some embodiments, the ecobiotic is SER-287 or a pharmaceutically acceptable salt thereof.

In some embodiments, the additional agent is a CD3 antagonist or pharmaceutically acceptable salt thereof. In some embodiments, the CD3 antagonist is NI-0401 or a pharmaceutically acceptable salt thereof.

In some embodiments, the additional agent is a F1F0 ATP synthase modulator or pharmaceutically acceptable salt thereof. In some embodiments, the F1F0 ATP synthase modulator is LYC-30937 EC or a pharmaceutically acceptable salt thereof.

In some embodiments, the additional agent is a glucocorticoid receptor modulator or pharmaceutically acceptable salt thereof. In some embodiments, the glucocorticoid receptor modulator is ABBV-3373 or a pharmaceutically acceptable salt thereof.

In some embodiments, the additional agent is a TNF ligand inhibitor or pharmaceutically acceptable salt thereof. In some embodiments, the TNF ligand inhibitor is ABBV-3373 or a pharmaceutically acceptable salt thereof.

In some embodiments, the additional agent is an ICAM1 gene inhibitor or pharmaceutically acceptable salt thereof. In some embodiments, the ICAM1 gene inhibitor is alicaforsen or a pharmaceutically acceptable salt thereof.

In some embodiments, the additional agent is an IL-18 antagonist or pharmaceutically acceptable salt thereof. In some embodiments, the IL-18 antagonist is GSK-1070806 or a pharmaceutically acceptable salt thereof.

In some embodiments, the additional agent is an IL-22 agonist or pharmaceutically acceptable salt thereof. In some embodiments, the IL-22 agonist is RG-7880 or a pharmaceutically acceptable salt thereof.

In some embodiments, the additional agent is an IL-22 agonist or pharmaceutically acceptable salt thereof. In some embodiments, the IL-23A inhibitor is guselkumab or a pharmaceutically acceptable salt thereof.

In some embodiments, the additional agent is an interleukin 1 like receptor 2 inhibitor or pharmaceutically acceptable salt thereof. In some embodiments, the interleukin 1 like receptor 2 inhibitor is BI-655130 or a pharmaceutically acceptable salt thereof.

In some embodiments, the additional agent is a lactoferrin stimulator or pharmaceutically acceptable salt thereof. In some embodiments, the lactoferrin stimulator is recombinant human lactoferrin (VEN-100) or a pharmaceutically acceptable salt thereof.

In some embodiments, the additional agent is a leukocyte elastase inhibitor or pharmaceutically acceptable salt thereof. In some embodiments, the leukocyte elastase inhibitor is tiprelestat or a pharmaceutically acceptable salt thereof.

In some embodiments, the additional agent is a leukocyte proteinase-3 inhibitor or pharmaceutically acceptable salt thereof. In some embodiments, the leukocyte proteinase-3 inhibitor is tiprelestat or a pharmaceutically acceptable salt thereof.

In some embodiments, the additional agent is a melanin concentrating hormone (MCH-1) antagonist or pharmaceutically acceptable salt thereof. In some embodiments, the melanin concentrating hormone (MCH-1) antagonist is CSTI-100 or a pharmaceutically acceptable salt thereof.

In some embodiments, the additional agent is a metalloprotease-9 inhibitor or pharmaceutically acceptable salt thereof. In some embodiments, the metalloprotease-9 inhibitor is GS-5745 or a pharmaceutically acceptable salt thereof.

In some embodiments, the additional agent is a NLRP3 inhibitor or pharmaceutically acceptable salt thereof. In some embodiments, the NLRP3 inhibitor is dapansutrile, BMS-986299, SB-414, MCC-950, IFM-514, JT-194, PELA-167, NBC-6, or a pharmaceutically acceptable salt thereof. In some embodiments, the NLRP3 inhibitor is dapansutrile or a pharmaceutically acceptable salt thereof. In some embodiments, the NLRP3 inhibitor is BMS-986299 or a pharmaceutically acceptable salt thereof. In some embodiments, the NLRP3 inhibitor is SB-414 or a pharmaceutically acceptable salt thereof. In some embodiments, the NLRP3 inhibitor is MCC-950 or a pharmaceutically acceptable salt thereof. In some embodiments, the NLRP3 inhibitor is IFM-514 or a pharmaceutically acceptable salt thereof. In some embodiments, the NLRP3 inhibitor is JT-194 or a pharmaceutically acceptable salt thereof. In some embodiments, the NLRP3 inhibitor is PELA-167 or a pharmaceutically acceptable salt thereof. In some embodiments, the NLRP3 inhibitor is NBC-6 or a pharmaceutically acceptable salt thereof.

In some embodiments, the additional agent is a farnesoid X receptor (FXR and NR1H4) agonist or modulator or pharmaceutically acceptable salt thereof. In some embodiments, the farnesoid X receptor (FXR and NR1H4) agonist or modulator is AGN-242266 or a pharmaceutically acceptable salt thereof. In some embodiments, the farnesoid X receptor (FXR and NR1H4) agonist or modulator is cilofexortromethamine (GS-9674) or a pharmaceutically acceptable salt thereof. In some embodiments, the farnesoid X receptor (FXR and NR1H4) agonist or modulator is EDP-305 or a pharmaceutically acceptable salt thereof. In some embodiments, the farnesoid X receptor (FXR and NR1H4) agonist or modulator is EYP-001 or a pharmaceutically acceptable salt thereof. In some embodiments, the farnesoid X receptor (FXR and NR1H4) agonist or modulator is EYP-001 or a pharmaceutically acceptable salt thereof. In some embodiments, the farnesoid X receptor (FXR and NR1H4) agonist or modulator is GNF-5120 or a pharmaceutically acceptable salt thereof. In some embodiments, the farnesoid X receptor (FXR and NR1H4) agonist or modulator is MET-409 or a pharmaceutically acceptable salt thereof. In some embodiments, the farnesoid X receptor (FXR and NR1H4) agonist or modulator is nidufexor (LMB-763) or a pharmaceutically acceptable salt thereof. In some embodiments, the farnesoid X receptor (FXR and NR1H4) agonist or modulator is obeticholic acid or a pharmaceutically acceptable salt thereof. In some embodiments, the farnesoid X receptor (FXR and NR1H4) agonist or modulator is TERN-101 or a pharmaceutically acceptable salt thereof. In some embodiments, the farnesoid X receptor (FXR and NR1H4) agonist or modulator is tropifexor or a pharmaceutically acceptable salt thereof.

In some embodiments, the additional agent is a nuclear factor kappa B inhibitor or pharmaceutically acceptable salt thereof. In some embodiments, the nuclear factor kappa B inhibitor is thetanix or a pharmaceutically acceptable salt thereof.

In some embodiments, the additional agent is an OX40 ligand inhibitor or pharmaceutically acceptable salt thereof. In some embodiments, the OX40 ligand inhibitor is KHK-4083 or a pharmaceutically acceptable salt thereof.

In some embodiments, the additional agent is a Pellino homolog 1 inhibitor or pharmaceutically acceptable salt thereof. In some embodiments, the Pellino homolog 1 inhibitor is BBT-401 or a pharmaceutically acceptable salt thereof.

In some embodiments, the additional agent is a Ret tyrosine kinase receptor inhibitor or pharmaceutically acceptable salt thereof. In some embodiments, the Ret tyrosine kinase receptor inhibitor is GSK-3179106 or a pharmaceutically acceptable salt thereof.

In some embodiments, the additional agent is a RIP-1 kinase inhibitor or pharmaceutically acceptable salt thereof. In some embodiments, the RIP-1 kinase inhibitor is GSK-2982772 or a pharmaceutically acceptable salt thereof.

In some embodiments, the additional agent is a RIP-2 kinase inhibitor or pharmaceutically acceptable salt thereof. In some embodiments, the RIP-2 kinase inhibitor is GSK-2983559 or a pharmaceutically acceptable salt thereof.

In some embodiments, the additional agent is a tissue transglutaminase inhibitor or pharmaceutically acceptable salt thereof. In some embodiments, the tissue transglutaminase inhibitor is zampilimab or a pharmaceutically acceptable salt thereof.

In some embodiments, the additional agent is a TLR-3 antagonist or pharmaceutically acceptable salt thereof. In some embodiments, the TLR-3 antagonist is PRV-300 or a pharmaceutically acceptable salt thereof.

In some embodiments, the additional agent is a TLR-4 antagonist or pharmaceutically acceptable salt thereof. In some embodiments, the TLR-4 antagonist is JKB-122 or a pharmaceutically acceptable salt thereof.

In some embodiments, the additional agent is a TLR8 inhibitor or pharmaceutically acceptable salt thereof. In some embodiments, the TLR8 is E-6887, IMO-4200, IMO-8400, IMO-9200, MCT-465, MEDI-9197, motolimod, resiquimod, VTX-1463, VTX-763, or a pharmaceutically acceptable salt thereof. In some embodiments, the TLR8 inhibitor is E-6887 or a pharmaceutically acceptable salt thereof. In some embodiments, the TLR8 inhibitor is IMO-4200 or a pharmaceutically acceptable salt thereof. In some embodiments, the TLR8 inhibitor is IMO-8400 or a pharmaceutically acceptable salt thereof. In some embodiments, the TLR8 inhibitor is IMO-9200 or a pharmaceutically acceptable salt thereof. In some embodiments, the TLR8 inhibitor is MCT-465 or a pharmaceutically acceptable salt thereof. In some embodiments, the TLR8 inhibitor is MEDI-9197 or a pharmaceutically acceptable salt thereof. In some embodiments, the TLR8 inhibitor is motolimod or a pharmaceutically acceptable salt thereof. In some embodiments, the TLR8 inhibitor is resiquimod or a pharmaceutically acceptable salt thereof. In some embodiments, the TLR8 inhibitor is VTX-1463 or a pharmaceutically acceptable salt thereof. In some embodiments, the TLR8 inhibitor is VTX-763 or a pharmaceutically acceptable salt thereof.

In some embodiments, the additional agent is a TLR-9 agonist or pharmaceutically acceptable salt thereof. In some embodiments, the TLR-9 agonist is cobitolimod, IMO-2055, IMO-2125, lefitolimod, litenimod, MGN-1601, PUL-042, or a pharmaceutically acceptable salt thereof. In some embodiments, the TLR-9 agonist is cobitolimod or a pharmaceutically acceptable salt thereof. In some embodiments, the TLR-9 agonist is IMO-2055 or a pharmaceutically acceptable salt thereof. In some embodiments, the TLR-9 agonist is IMO-2125 or a pharmaceutically acceptable salt thereof. In some embodiments, the TLR-9 agonist is lefitolimod or a pharmaceutically acceptable salt thereof. In some embodiments, the TLR-9 agonist is litenimod or a pharmaceutically acceptable salt thereof. In some embodiments, the TLR-9 agonist is MGN-1601 or a pharmaceutically acceptable salt thereof. In some embodiments, the TLR-9 agonist is PUL-042 or a pharmaceutically acceptable salt thereof.

In some embodiments, the additional agent is a TPL-2 inhibitor or pharmaceutically acceptable salt thereof. In some embodiments, the TPL-2 inhibitor is GS-4875 or a pharmaceutically acceptable salt thereof.

In some embodiments, the additional agent is a tumor necrosis factor superfamily member 15 inhibitor or pharmaceutically acceptable salt thereof. In some embodiments, the tumor necrosis factor superfamily member 15 inhibitor is PF-06480605, PRA023, or a pharmaceutically acceptable salt thereof. In some embodiments, the tumor necrosis factor superfamily member 15 inhibitor is PF-06480605 or a pharmaceutically acceptable salt thereof. In some embodiments, the tumor necrosis factor superfamily member 15 inhibitor is PRA023 or a pharmaceutically acceptable salt thereof.

In some embodiments, the additional agent is a Tyk2 tyrosine kinase inhibitor or pharmaceutically acceptable salt thereof. In some embodiments, the Tyk2 tyrosine kinase inhibitor is PF-06826647, BMS-986165, or a pharmaceutically acceptable salt thereof. In some embodiments, the Tyk2 tyrosine kinase inhibitor is PF-06826647 or a pharmaceutically acceptable salt thereof. In some embodiments, the Tyk2 tyrosine kinase inhibitor is BMS-986165 or a pharmaceutically acceptable salt thereof.

In some embodiments, the additional agent is a Type I IL-1 receptor antagonist or pharmaceutically acceptable salt thereof. In some embodiments, the Type I IL-1 receptor antagonist is anakinra or a pharmaceutically acceptable salt thereof.

In some embodiments, the additional agent is an anti-CD28 inhibitor or pharmaceutically acceptable salt thereof. In some embodiments, the anti-CD28 inhibitor is JNJ-3133, abatacept, or a pharmaceutically acceptable salt thereof. In some embodiments, the anti-CD28 inhibitor is JNJ-3133 or a pharmaceutically acceptable salt thereof. In some embodiments, the anti-CD28 inhibitor is abatacept or a pharmaceutically acceptable salt thereof.

In some embodiments, the additional agent is a Type I IL-1 receptor antagonist or pharmaceutically acceptable salt thereof. In some embodiments, the Type I IL-1 receptor antagonist is anakinra or a pharmaceutically acceptable salt thereof.

In some embodiments, the additional agent is a CD4 antagonist or pharmaceutically acceptable salt thereof. In some embodiments, the CD4 antagonist is IT-1208 or a pharmaceutically acceptable salt thereof.

In some embodiments, the additional agent is a CD40 gene inhibitor or pharmaceutically acceptable salt thereof. In some embodiments, the CD40 gene inhibitor is NJA-730 or a pharmaceutically acceptable salt thereof.

In some embodiments, the additional agent is a chaperonin binding immunoglobulin protein or pharmaceutically acceptable salt thereof. In some embodiments, the chaperonin binding immunoglobulin protein is IRL-201805 or a pharmaceutically acceptable salt thereof.

In some embodiments, the additional agent is a COT protein kinase inhibitor or pharmaceutically acceptable salt thereof. In some embodiments, the COT protein kinase inhibitor is GS-4875 or a pharmaceutically acceptable salt thereof.

In some embodiments, the additional agent is a glucocorticoid receptor agonist or pharmaceutically acceptable salt thereof. In some embodiments, the glucocorticoid receptor agonist is budesonide, beclomethasone dipropionate, dexamethasone sodium phosphate, or a pharmaceutically acceptable salt thereof. In some embodiments, the glucocorticoid receptor agonist is budesonide or a pharmaceutically acceptable salt thereof. In some embodiments, the glucocorticoid receptor agonist is beclomethasone dipropionate or a pharmaceutically acceptable salt thereof. In some embodiments, the glucocorticoid receptor agonist is dexamethasone sodium phosphate or a pharmaceutically acceptable salt thereof.

In some embodiments, the additional agent is a HIF prolyl hydroxylase inhibitor or pharmaceutically acceptable salt thereof. In some embodiments, the HIF prolyl hydroxylase inhibitor is DS-1093, AKB-4924, or a pharmaceutically acceptable salt thereof. In some embodiments, the HIF prolyl hydroxylase inhibitor is DS-1093 or a pharmaceutically acceptable salt thereof. In some embodiments, the HIF prolyl hydroxylase inhibitor is AKB-4924 or a pharmaceutically acceptable salt thereof.

In some embodiments, the additional agent is a histone deacetylase-6 inhibitor or pharmaceutically acceptable salt thereof. In some embodiments, the histone deacetylase-6 inhibitor is CKD-506 or a pharmaceutically acceptable salt thereof.

In some embodiments, the additional agent is a neuregulin-4 ligand or pharmaceutically acceptable salt thereof. In some embodiments, the neuregulin-4 ligand is NRG-4 or a pharmaceutically acceptable salt thereof.

In some embodiments, the additional agent is a microbiome modulator or pharmaceutically acceptable salt thereof. In some embodiments, the microbiome modulator is ABI-M201 or a pharmaceutically acceptable salt thereof.

In some embodiments, the additional agent is a TrkA receptor antagonist or pharmaceutically acceptable salt thereof. In some embodiments, the TrkA receptor antagonist is SNA-125 or a pharmaceutically acceptable salt thereof.

In some embodiments, the additional agent is a JAK inhibitor. In some embodiments, the JAK inhibitor is AT9283, AZD1480, baricitinib, BMS-911543, fedratinib, filgotinib (GLPG0634), gandotinib (LY2784544), INCB039110, lestaurtinib, momelotinib (CYT0387), NS-018, pacritinib (SB1518), peficitinib (ASPO15K), ruxolitinib, tofacitinib, XLO19, upadacitinib (ABT-494), filgotinib, GLPG-0555, SHR-0302, brepocitinib (PF-06700841), or a pharmaceutically acceptable salt thereof. In some embodiments, the JAK inhibitor is AT9283 or a pharmaceutically acceptable salt thereof. In some embodiments, the JAK inhibitor is AZD1480 or a pharmaceutically acceptable salt thereof. In some embodiments, the JAK inhibitor is baricitinib or a pharmaceutically acceptable salt thereof. In some embodiments, the JAK inhibitor is BMS-911543 or a pharmaceutically acceptable salt thereof. In some embodiments, the JAK inhibitor is fedratinib or a pharmaceutically acceptable salt thereof. In some embodiments, the JAK inhibitor is filgotinib(GLPG0634) or a pharmaceutically acceptable salt thereof. In some embodiments, the JAK inhibitor is gandotinib (LY2784544) or a pharmaceutically acceptable salt thereof. In some embodiments, the JAK inhibitor is INCB039110 or a pharmaceutically acceptable salt thereof. In some embodiments, the JAK inhibitor is lestaurtinib or a pharmaceutically acceptable salt thereof. In some embodiments, the JAK inhibitor is momelotinib (CYT0387) or a pharmaceutically acceptable salt thereof. In some embodiments, the JAK inhibitor is NS-018 or a pharmaceutically acceptable salt thereof. In some embodiments, the JAK inhibitor is pacritinib (SB1518) or a pharmaceutically acceptable salt thereof. In some embodiments, the JAK inhibitor is ruxolitinib or a pharmaceutically acceptable salt thereof. In some embodiments, the JAK inhibitor is tofacitinib or a pharmaceutically acceptable salt thereof. In some embodiments, the JAK inhibitor is XLO19 or a pharmaceutically acceptable salt thereof. In some embodiments, the JAK inhibitor is upadacitinib (ABT-494) or a pharmaceutically acceptable salt thereof. In some embodiments, the JAK inhibitor is filgotinib or a pharmaceutically acceptable salt thereof. In some embodiments, the JAK inhibitor is GLPG-0555 or a pharmaceutically acceptable salt thereof. In some embodiments, the JAK inhibitor is SHR-0302 or a pharmaceutically acceptable salt thereof. In some embodiments, the JAK inhibitor is brepocitinib (PF-06700841) or a pharmaceutically acceptable salt thereof.

In some embodiments, the additional agent isobefazimod (ABX-4640), adalimumab, alicaforsen, ALLO-ASC-CD, AMG-966, anakinra, apremilast, Alequel, AMG-139, amiselimod, ASD-003, ASP-3291, AX-1505, BBT-401, balsalazide, beclomethasone dipropionate, BI-655130, BMS-986184, budesonide, CEQ-508, certolizumab, ChAdOx2-HAV, dexamethasone sodium phosphate, DNVX-078, etanercept, cibinetide, Clostridium butyricum, ETX-201, golimumab, GS-4997, GS-9876, GS-4875, GS-4059, infliximab, mesalazine, HLD-400, LYC-30937 EC, IONIS-JB11-2.5Rx, JNJ-64304500, JNJ-4447, naltrexone, natalizumab, neihulizumab, olsalazine, PH-46-A, propionyl-L-carnitine, PTG-100, remestemcel-L, tacrolimus, teduglutide, tofacitinib, ASP-1002, ustekinumab, vedolizumab, AVX-470, INN-108, SGM-1019, PF-06480605, PF-06651600, PF-06687234, RBX-8225, SER-287, Thetanix, TOP-1288, VBY-129, 99mTc-annexin V-128, bertilimumab, DLX-105, dolcanatide, FFP-104, filgotinib, foralumab, GED-0507-34-Levo, givinostat, GLPG-0974, iberogast, JNJ-40346527, K(D)PT, KAG-308, KHK-4083, KRP-203, larazotide acetate, LY-3074828, midismase, olokizumab, OvaSave, P-28-GST, PF-547659, prednisolone, QBECO, RBX-2660, RG-7835, JKB-122, SB-012, STNM-01, Debio-0512, TRK-170, zucapsaicin, ABT-494, Ampion, BI-655066, carotegast methyl, cobitolimod, elafibranor, etrolizumab, GS-5745, HMPL-004, LP-02, ozanimod, peficitinib, quetmolimab (E-6011), RHB-104, rifaximin, tildrakizumab, tralokinumab, brodalumab, laquinimod, plecanatide, vidofludimus, AZD-058, or a pharmaceutically acceptable salt thereof.

In some embodiments, the JAK inhibitor is a compound as disclosed in U.S. Pat. No. 9,233,934B2.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an agent for treating metabolic disorders. These agents include pancreatic lipase inhibitors (e.g., orlistat); insulin; insulin sensitizers, including biguanides (e.g., buformin, metformin, and phenformin) and glitazones (e.g., pioglitazone and rosiglitazone); insulin secretagogues, including sulfonylureas (e.g., acetohexamide, chlorpropamide, tolazamide, tolbutamide, gliclazide, glimepiride, glipizide, and glyburide), and meglitinides (e.g., nateglinide and repaglinide); alpha-glucosidase inhibitors (e.g., acarbose and miglitol); glucagon-like peptide analogs and agonists (e.g., exenatide, liraglutide, and taspoglutide); dipeptidyl peptidase-4 inhibitors (e.g., alogliptin, linagliptin, saxagliptin, sitagliptin, and vildagliptin); and amylin analogs (e.g., pramlinitide).

In some embodiments, the additional therapeutic agent is an agent for treating wound healing disorders. In some embodiments, the additional therapeutic agent is an anti-inflammatory agent, analgesics, an antipruritic, or an anti-infective.

Examples of anti-inflammatory agents include nonsteroidal anti-inflammatory drugs (NSAIDs) and corticosteroids. Representative NSAIDs include apazone, aspirin, celecoxib, diclofenac (with and without misoprostol), diflunisal, etodolac, fenoprofen, flurbiprofen, ibuprofen, indomethacin, ketoprofen, meclofenamate sodium, mefenamic acid, meloxicam, nabumetone, naproxen, oxaprozin, phenylbutazone, piroxicam, choline and magnesium salicylates, salsalate, and sulindac. Representative corticosteroids include betamethasone, cortisone acetate, dexamethasone, hydrocortisone, methylprednisolone, prednisolone, and prednisone. Representative analgesics include acetaminophen and morphine sulfate, as well as codeine, hydrocodone, oxycodone, propoxyphene, and tramadol, all with or without acetaminophen. Representative antipruritics for systemic use include cyproheptadine, diphenhydramine, gabapentin, hydroxyzine, and ondansetron.

Example anti-infective agents may include antibacterials, antifungals, and antivirals.

Representative antibacterials include aminoglycosides, such as amikacin, gentamicin, kanamycin, neomycin, paromomycin, and tobramycin; carbapenems, such as doripenem, ertapenem, imipenem, and meropenem; cephalosporins, including combinations with beta-lactamase inhibitors such as ceftazidime/avibactam and ceftolozane/tazobactam; first-generation cephalosporins, such as cefadroxil, cefazolin, cephalexin, and cephradine; second-generation cephalosporins, such as cefotetan, cefprozil, cefuroxime, efoxitin, and loracarbef; third-generation cephalosporins, such as cefdinir, cefditoren, cefixime, cefoperazone, cefotaxime, cefpodoxime, ceftazidime, ceftibuten, ceftizoxime, and ceftriaxone; and fourth- and next-generation cephalosporins, such as cefepime and ceftaroline; glycopeptide antibiotics, such as dalbavancin, oritavancin, telavancin, and vancomycin; glycylcyclines, such as tigecycline; lincomycin and its derivatives, such as clindamycin; macrolides, such as azithromycin, clarithromycin, erythromycin, and fidaxomicin, and macrolide derivatives, including ketolides such as telithromycin; oxazolidinone antibiotics, such as linezolid and tedizolid; penicillins, including aminopenicillins, such as amoxicillin and ampicillin; antipseudomonal penicillins, such as carbenicillin, piperacillin, and ticarcillin; penicillins with beta-lactamase inhibitors such as amoxicillin/clavulanate, ampicillin/sulbactam, piperacillin/tazobactam, and ticarcillin/clavulanate; natural penicillins, such as penicillin G benzathine, penicillin V potassium, and procaine penicillin; penicillinase resistant penicillins, such as dicloxacillin, nafcillin, and oxacillin; quinolones, such as cinoxacin, ciprofloxacin, delafloxacin, gatifloxacin, gemifloxacin, levofloxacin, lomefloxacin, moxifloxacin, nalidixic acid, norfloxacin, ofloxacin, sparfloxacin, and trovafloxacin; sulfonamides, such as sulfamethoxazole/trimethoprim and sulfisoxazole; tetracycline and its derivatives, such as demeclocycline, doxycycline, doxycycline/omega-3 polyunsaturated fatty acids, doxycycline/salicylic acid, minocycline, and oxytetracycline. Other representative antibacterials include atovaquone, aztreonam, bacitracin, chloramphenicol, colistimethate, dalfopristin/quinupristin, daptomycin, erythromycin/sulfisoxazole, fosfomycin, metronidazole, pentamidine, rifaximin, spectinomycin, and trimetrexate.

Representative antifungals include azole antifungals, such as clotrimazole, fluconazole, isavuconazonium, itraconazole, ketoconazole, miconazole, posaconazole, and voriconazole; echinocandins, such as anidulafungin, caspofungin, and micafungin; and polyenes, such as amphotericin B, amphotericin B cholesteryl sulfate, amphotericin B lipid complex, and nystatin. Other representative antifungals include flucytosine, griseofulvin, and terbinafine.

Representative antiviral agents include purine nucleosides, such as acyclovir, cidofovir, famciclovir, ganciclovir, ribavirin, valacyclovir, and valganciclovir.

In some embodiments, the additional therapeutic agent is an anti-cancer agent. In some embodiments, the additional therapeutic agent is a chemotherapeutic agent (i.e., cytotoxic, or antineoplastic agents) such as alkylating agents, antibiotics, antimetabolic agents, plant-derived agents, and topoisomerase inhibitors, as well as molecularly targeted drugs which block the growth and spread of cancer by interfering with specific molecules involved in tumor growth and progression. Molecularly targeted drugs include both small molecules and biologics.

Representative alkylating agents include bischloroethylamines (nitrogen mustards) including chlorambucil, cyclophosphamide, ifosfamide, mechlorethamine, melphalan, and uracil mustard); aziridines, including thiotepa; alkyl alkone sulfonates, including busulfan; nitrosoureas, including carmustine, lomustine, and streptozocin; nonclassical alkylating agents, including altretamine, dacarbazine, and procarbazine; and platinum compounds, including carboplatin, cisplatin, nedaplatin, oxaliplatin, satraplatin, and triplatin tetranitrate.

Representative antibiotic agents include anthracyclines, including aclarubicin, amrubicin, daunorubicin, doxorubicin, epirubicin, idarubicin, pirarubicin, valrubicin, and zorubicin; anthracenediones, including mitoxantrone and pixantrone; and Streptomyces, including actinomycin, bleomycin, dactinomycin, mitomycin C, and plicamycin.

Representative antimetabolic agents include dihydrofolate reductase inhibitors, including aminopterin, methotrexate, and pemetrexed; hymidylate synthase inhibitors, including raltitrexed and pemetrexed; folinic acid, including leucovorin; adenosine deaminase inhibitors, including pentostatin; halogenated/ribonucleotide reductase inhibitors, including cladribine, clofarabine, and fludarabine; thiopurines, including thioguanine and mercaptopurine; thymidylate synthase inhibitors, including fluorouracil, capecitabine, tegafur, carmofur, and floxuridine; DNA polymerase inhibitors, including cytarabine; ribonucleotide reductase inhibitors, including gemcitabine; hypomethylating agent, including azacitidine and decitabine; ribonucleotide reductase inhibitor, including hydroxyurea; and an asparagine deplete, including asparaginase.

Representative plant-derived agents include vinca alkaloids, including vincristine, vinblastine, vindesine, vinzolidine, and vinorelbine; podophyllotoxins, including etoposide and teniposide; and taxanes, including docetaxel, larotaxel, ortataxel, paclitaxel, and tesetaxel.

Representative type I topoisomerase inhibitors include camptothecins, including belotecan, irinotecan, rubitecan, and topotecan. Representative type II topoisomerase inhibitors include amsacrine, etoposide, etoposide phosphate, and teniposide, which are derivatives of epipodophyllotoxins.

Molecularly targeted therapies include biologic agents such as cytokines and other immune-regulating agents. Useful cytokines include interleukin-2 (IL-2, aldesleukin), interleukin 4 (IL-4), interleukin 12 (IL-12), and interferon, which includes more than 23 related subtypes. Other cytokines include granulocyte colony stimulating factor (CSF) (filgrastim) and granulocyte macrophage CSF (sargramostim). Other immuno-modulating agents include bacillus Calmette-Guerin, levamisole, and octreotide; monoclonal antibodies against tumor antigens, such as trastruzumab and rituximab; and cancer vaccines, which induce an immune response to tumors.

In addition, molecularly targeted drugs that interfere with specific molecules involved in tumor growth and progression include inhibitors of epidermal growth factor (EGF), transforming growth factor-alpha (TGFₐ), TGFp, heregulin, insulin-like growth factor (IGF), fibroblast growth factor (FGF), keratinocyte growth factor (KGF), colony stimulating factor (CSF), erythropoietin (EPO), interleukin-2 (IL-2), nerve growth factor (NGF), platelet-derived growth factor (PDGF), hetaptocyte growth factor (HGF), vascular endothelial growth factor (VEGF), angiopoietin, epidermal growth factor receptor (EGFR), human epidermal growth factor receptor 2 (HER2), HER4, insulin-like growth factor 1 receptor (IGF1R), IGF2R, fibroblast growth factor 1 receptor (FGFIR), FGF2R, FGF3R, FGF4R, vascular endothelial growth factor receptor (VEGFR), tyrosine kinase with immunoglobulin-like and epidermal growth factor-like domains 2 (Tie-2), platelet-derived growth factor receptor (PDGFR), Abl, Bcr-Abl, Raf, FMS-like tyrosine kinase 3 (FLT3), c-Kit, Src, protein kinase c (PKC), tropomyosin receptor kinase (Trk), Ret, mammalian target of rapamycin (mTOR), Aurora kinase, polo-like kinase (PLK), mitogen activated protein kinase (MEK), mesenchymal-epithelial transition factor (c-MET), cyclin-dependent kinase (CDK), Akt, extracellular signal-regulated kinases (ERK), poly(ADP) ribose polymerase (PARP), and the like.

Specific molecularly targeted drugs include selective estrogen receptor modulators, such as tamoxifen, toremifene, fulvestrant, and raloxifene; antiandrogens, such as bicalutamide, nilutamide, megestrol, and flutamide; and aromatase inhibitors, such as exemestane, anastrozole, and letrozole. Other specific molecularly targeted drugs include agents which inhibit signal transduction, such as imatinib, dasatinib, nilotinib, trastuzumab, gefitinib, erlotinib, cetuximab, lapatinib, panitumumab, and temsirolimus; agents that induce apoptosis, such as bortezomib; agents that block angiogenesis, such as bevacizumab, sorafenib, and sunitinib; agents that help the immune system destroy cancel cells, such as rituximab and alemtuzumab; and monoclonal antibodies which deliver toxic molecules to cancer cells, such as gemtuzumab ozogamicin, tositumomab, 1311-tositumoab, and ibritumomab tiuxetan.

### Method of Treatment

Disclosed herein is a method of treating a disease or disorder in a subject, the method comprising administering to the subject a compound disclosed herein, or a pharmaceutically acceptable salt, or stereoisomer thereof, wherein the disease or disorder is inflammatory epithelial disease. In some embodiments, the disease or disorder is inflammatory bowel disease (IBD). In some embodiments, the disease or disorder is ulcerative colitis (“UC”) or Crohn's disease (“CD”). In some embodiments, the disease or disorder is ulcerative colitis (“UC”). In some embodiments, the disease or disorder is Crohn's disease (“CD”).

### Inflammatory Bowel Disease (IBD)

IBD is an umbrella term used to describe disorders that involve chronic inflammation of the digestive tract. Types of IBD include ulcerative colitis (“UC”) and Crohn's disease (“CD”). IBD symptoms vary and depend on the severity of inflammation and the location it occurs. According to GlobalData, in 2019, there were 1.7 million diagnosed UC patients in 8 major markets (US, 5EU, Japan and Canada) and the market sales reached $6.8 billion in that year. [In addition, there were 1.3 million UC diagnosed prevalent population in 8 major markets (US, 5EU, Japan and Canada) and the market sales reach $7.4 billion.]

Inflammatory bowel diseases are characterized by repeated inflammation and wounding of the mucosa and loss of the intestinal epithelial barrier function, which lead to the passage of bacteria or bacterial products from the gut lumen to the serosa and into the blood, resulting in systemic bacteremia and endotoxemia. PHD inhibition has been shown to reduce disease severity in murine models of colitis on several levels of clinical scoring. The proposed mechanism for the therapeutic activity of PHD inhibitors is through HIF-1a stabilization, which drives epithelial barrier augmentation and healing.

Despite the efficacy of current treatment with anti-inflammation agents or immune-suppressive agents, a large fraction of IBD patients do not respond adequately to currently available therapies and do not achieve long-term remission. Inhibitors of PHDs may provide a new therapeutic option for IBD and may be combined with available anti-inflammatory drugs to achieve an enhanced efficacy.

In some embodiments, the inflammatory epithelial disease is a disease affecting the respiratory tract, mucosa, skin, gastrointestinal tract, lining of major organs and endocrine glands, vascular tissue, or any combination thereof. In some embodiments, the inflammatory epithelial disease is a disease affecting the respiratory tract. In some embodiments, the inflammatory epithelial disease is a disease affecting the mucosa. In some embodiments, the inflammatory epithelial disease is a disease affecting the skin. In some embodiments, the inflammatory epithelial disease is a disease affecting the gastrointestinal tract. In some embodiments, the inflammatory epithelial disease is a disease affecting the lining of major organs. In some embodiments, the inflammatory epithelial disease is a disease affecting the lining of endocrine glands. In some embodiments, the inflammatory epithelial disease is a disease affecting the vascular tissue.

In some embodiments, the inflammatory epithelial disease is ulcerative colitis, Crohn's disease, collagenous colitis, lymphocytic colitis, ischemic colitis, diversion colitis, Beheet's syndrome, or indeterminate colitis. In some embodiments, the inflammatory epithelial disease is ulcerative colitis. In some embodiments, the inflammatory epithelial disease is Crohn's disease. In some embodiments, the inflammatory epithelial disease is collagenous colitis. In some embodiments, the inflammatory epithelial disease is lymphocytic colitis. In some embodiments, the inflammatory epithelial disease is ischemic colitis. In some embodiments, the inflammatory epithelial disease is diversion colitis. In some embodiments, the inflammatory epithelial disease is Behcet's syndrome. In some embodiments, the inflammatory epithelial disease is indeterminate colitis.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In some embodiments, the administration is oral administration. In some embodiments, the administration is intracolonic.

### Pharmaceutical Compositions/Formulations

The compounds or agents described herein are administered to a subject in need thereof, either alone or in combination with pharmaceutically acceptable carriers, excipients, or diluents, in a pharmaceutical composition, according to standard pharmaceutical practice. In one embodiment, the compounds may be administered to animals.

In another aspect, provided herein are pharmaceutical compositions comprising a compound described herein, or a pharmaceutically acceptable salt, or stereoisomer thereof, and at least one pharmaceutically acceptable excipient. Pharmaceutical compositions are formulated in a conventional manner using one or more pharmaceutically acceptable excipients that facilitate processing of the active compounds or agents into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds or agents described herein, or a pharmaceutically acceptable salt, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds or agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds or agents are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

All compounds can be synthesized according to the methods disclosed in WO2023072257, which is hereby incorporated by reference in its entirety.

### Example A.1: Combination of Compound 29 and 5-Aminosalicylates (5-ASAs) in 2,4,6-Trinitrobenzenesulfonic Acid (TNBS) Induced Inflammatory Bowel Disease (IBD) Model

### Materials and Methods

Female BALB/c mice aged 8 weeks, around 18-20 g body weight purchased from Charles River Laboratories were bred in specific pathogen free IVC cages (4 mice in each cage) in a temperature controlled (20±2° C.) room with a 12 hour light-dark cycle. Chow pellets and tap water were available ad libitum. Mice in sham group, intracolonic injection 50% ethanol (0.1 mL) at day 0, whereas mice in other four TNBS colitis model groups, intracolonic injection of 2% TNBS solution (0.1 mL) at day 0. These mice were euthanized on day 8 (10th day of the experiment), and samples were immediately harvested for the last endpoint.

The mice were divided into 5 groups with 10 mice in each group. The dosing frequency for mice in Mesalamine group were once daily given Mesalamine in 0.5% CMC sodium by gavage from Day-1 to Day 7, whereas the mice in the vehicle group were given 0.5% carboxymethyl cellulose (CMC) and 2% tween 80 in water by gavage once daily. The mice were dosed with Compound 29 in 0.5% carboxymethyl cellulose (CMC) and 2% tween 80 in water by gavage. The dose regimen and group setting are shown in Table 2.

TABLE 2
Grouping and Dosing Regimen for TNBS
induced IBD Model for in-life study

| | | | Treatment | Systematic dosing | |
|---|---|---|---|---|---|
| Group | N | Test articles | (mg/kg) | Route | Frequency |
| 1 | 10 | Sham | NA | PO | Once daily (QD) |
| 2 | 10 | Vehicle | NA | PO | Once daily (QD) |
| 3 | 10 | Mesalamine | 25 | PO | Once daily (QD) |
| 4 | 10 | Compound 29 | 3 | PO | Once daily (QD) |
| 5 | 10 | Mesalamine+ | 25 + 3 | PO | Once daily (QD) |
| | | Compound 29 | | | |

The animals will be monitored for weight and DAI scores daily. At the endpoint, serum can be chosen for cytokines examination, and the colon tissues can be chosen to Swiss-rolled and PFA fixed for the histology study.

Body weight and disease activity index (DAI) scoring were recorded daily to assess the colitis. DAI score was the sum of the weight loss score, stool score and bleeding subscores. A blinded scoring system was employed to assess the colitis. The DAI scorer blinded to the group information and animal ID was responsible for the stool consistency and bleeding evaluations. DAI scoring standards will be followed as Table 3.

TABLE 3
DAI scoring system

| Score | Weight loss | Stool consistency | Bleeding |
|---|---|---|---|
| 0 | no loss | Normal | Occult blood negative |
| 1 | 1~5% | Soft but still formed | Occult blood weak positive |
| 2 | 6~10% | Very soft | Occult blood positive |
| 3 | 11~20% | Diarrhea | Bleeding |
| 4 | >20% | Watery diarrhea | Bleeding heavily |

If there was no blood visible with naked eyes, the Fecal occult blood (FOB) test was performed with a fecal occult blood test strip (the improved pyramidon method). The FOB score (0-2) was interpreted as follows: 0, no color appeared after 2 min; 1, a dimmed color during 1-2 min; 2, a deeper color during 1-2 min.

The colons were harvested, mesentery and adipose tissue were carefully removed. The colons were arranged on the ruled paper for photograph. After that, the length of colon was measured, and then colon weight was measured by removing and rinsing out the internal content of colon with cold PBS. The colon index as a marker of tissue edema was obtained by calculation of colon weight/length ratio.

### Results

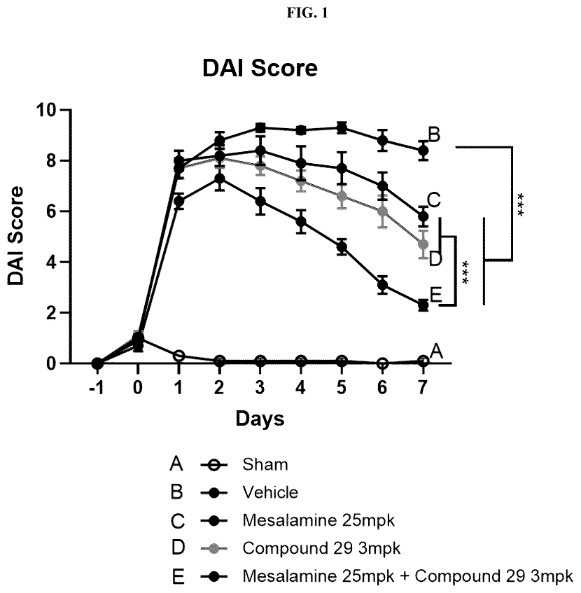

As seen in FIG. 1, the mice group that received Mesalamine (Line C) showed a therapeutic effect with a decrease of DAI score over the following days. The mice group that received Compound 29 (Line D) showed a quicker decrease in DAI score compared to Mesalamine. The mice group that received the combination of Mesalamine and Compound 29 (Line E) showed a larger decrease in DAI score over the following days. Sham (Line A) and Vehicle (Line B) were used as controls.

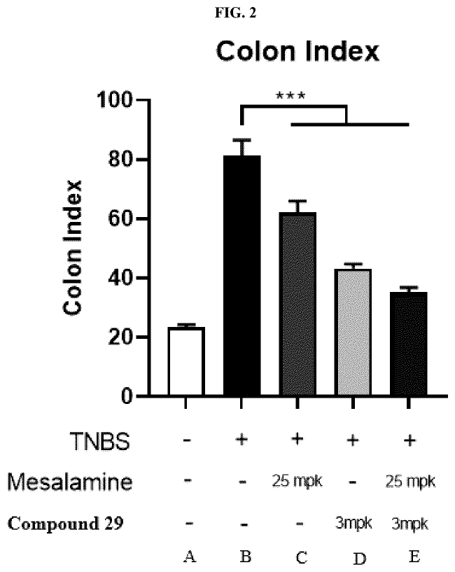

As seen in FIG. 2, the mice group that received Mesalamine (Bar C) had colon index around 60. The mice group that received Compound 29 (Bar D) had colon index above 40. The mice group that received the combination of Mesalamine and Compound 29 (Bar E) had colon index below 40. Sham (Bar A) and Vehicle (Bar B) were used as controls.

### Example A.2: Example of Compound 29 Combination with Cyclosporine in Dextran Sodium Sulfate (DSS) Induced IBD Model

### Methods and Materials

Female C57 BL/6J mice aged 8 weeks, around 18-20 g body weight purchased from Charles River Laboratories were bred in specific pathogen free IVC cages (5 mice in each cage) in a temperature controlled (20±2° C.) room with a 12 hour light-dark cycle. Chow pellets and tap water were available ad libitum. To generate dextran sodium sulfate (DSS)-induced acute colitis, the mice were induced with 3% DSS (molecular weight 36,000-50,000; MP Biomedicals, Santa Ana, CA, USA) in their drinking water for 7 consecutive days, and then switched to normal water. DSS solution was freshly prepared and changed daily. These mice were euthanized on day 8 (9th day of the experiment), and samples were immediately harvested for the last endpoint.

The Naive group was given the blank drinking water, and the other 4 model groups were given 3% DSS for seven consecutive days (Day 0-Day 6). The dosing frequency for mice in CsA group were once daily given cyclosporine A (CsA) in physical saline by gavage, whereas the mice in the vehicle group were given 0.5% CMC-Na by gavage once daily. The mice from group 4 and 5 were dosed with Compound 29 in 0.5% CMC-Na by gavage. The dose regimen and group setting are shown in Table 4.

TABLE 4
Grouping and Dosing Regimen for DSS
induced IBD Model for in-life study

| | | | Treatment | Dosing | |
|---|---|---|---|---|---|
| Group | N | Test articles | (mg/kg) | Route | Frequency |
| 1 | 10 | Naïve (no DSS) | NA | NA | NA |
| 2 | 10 | Vehicle | NA | PO | Once daily (QD) |
| 3 | 10 | CsA | 20 | PO | Once daily (QD) |
| 4 | 10 | Compound 29 | 3 | PO | Once daily (QD) |
| 5 | 10 | CsA + | 20 + 3 | PO | Once daily (QD) |
| | | Compound 29 | | | |

Body weight and DAI soring were recorded daily to assess the colitis. DAI score was the sum of the weight loss score, stool score and bleeding subscores. A blinded scoring system was employed to assess the colitis. The DAI scorer blinded to the group information and animal ID was responsible for the stool consistency and bleeding evaluations. DAI scoring standards as in Table 5 were followed.

If there was no blood visible with naked eyes, the Fecal occult blood (FOB) test was performed with a fecal occult blood test strip (the improved pyramidon method). The FOB score (0-2) was interpreted as follows: 0, no color appeared after 2 min; 1, a dimmed color during 1-2 min; 2, a deeper color during 1-2 min.

The colons were harvested, mesentery and adipose tissue were carefully removed. The colons were arranged on paper for photograph. After that, the length of colon was measured, and then colon weight was measured by removing and rinsing out the internal content of colon with cold PBS. The colon index as a marker of tissue edema was obtained by calculation of colon weight/length ratio.

### Results.

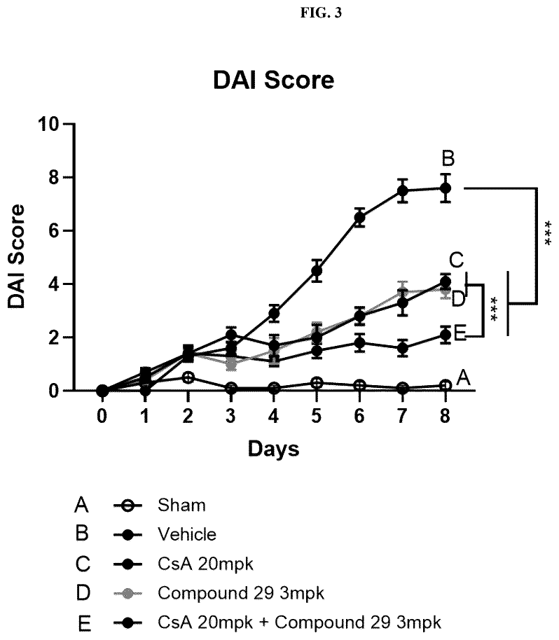

As seen in FIG. 3, the mice group that received CsA (Line C) showed a therapeutic effect with a decrease of DAI score over the following days. The mice group that received Compound 29 (Line D) showed a decrease in DAI. The mice group that received the combination of CsA and Compound 29 (Line E) showed a larger decrease in DAI score over the following days. Sham (Line A) and Vehicle (Line B) were used as controls.

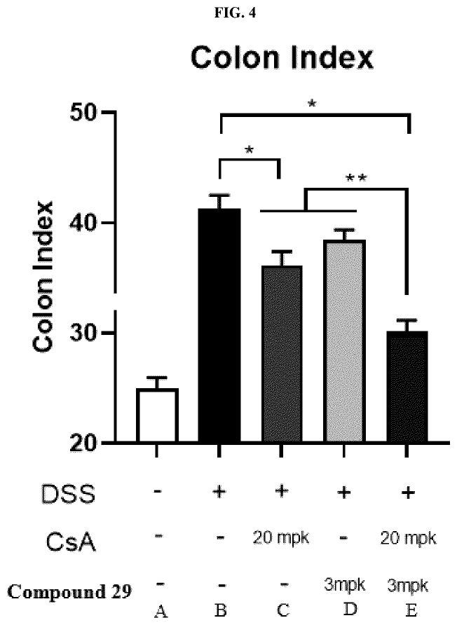

As seen in FIG. 4, the mice group that received CsA (Bar C) had colon index around 35. The mice group that received Compound 29 (Bar D) had colon index less than 40. The mice group that received the combination of CsA and Compound 29 (Bar E) had colon index around 30. Sham (Bar A) and Vehicle (Bar B) were used as controls.

### Example A.3: Example of Compound 29 Combination with Anti-TNFα Antibody (Etanercept, Manufactured by Boehringer Ingelheim) in Dextran Sodium Sulfate (DSS) Induced IBD Model

### Methods and Materials

Female BALB/c mice will be grouped into 5 groups, 10 mice in each group: mice in sham group, intracolonic injection 50% ethanol (0.1 mL) at day 0, whereas mice in other six colitis model groups, intracolonic injection of 2% TNBS solution (0.1 mL) at day 0. The mice will be dosing from day-1 to day 6, and day 7 is the endpoint. The dose regimen and group setting are shown in Table 5.

TABLE 5
Grouping and Dosing Regimen for TNBS
induced IBD Model for in-life study

| | | | Treatment | Systematic dosing | |
|---|---|---|---|---|---|
| Group | N | Test articles | (mg/kg) | Route | Frequency |
| 1 | 10 | Sham | NA | PO | Once daily (QD) |
| 2 | 10 | Vehicle | NA | PO | Once daily (QD) |
| 3 | 10 | Anti-TNFα | 20 | PO | Once daily (QD) |
| | | antibody | | | |
| 4 | 10 | Compound 29 | 3 | PO | Once daily (QD) |
| 5 | 10 | Anti-TNFα | 20 + 3 | PO | Once daily (QD) |
| | | antibody + | | | |
| | | Compound 29 | | | |

The animals will be monitored for weight and DAI scores daily. At the endpoint, The colons were harvested, mesentery and adipose tissue were carefully removed. The colons were arranged on paper for photograph. After that, the length of colon was measured, and then colon weight was measured by removing and rinsing out the internal content of colon with cold PBS. The colon index as a marker of tissue edema was obtained by calculation of colon weight/length ratio.

Compound 29 will be dissolved in 0.5% carboxymethyl cellulose sodium (CMC-Na) and 2% tween 80, and anti-TNFα was diluted with DPBS and prepared once per 3 days, stored at 4° C. to keep the stability.

### Results

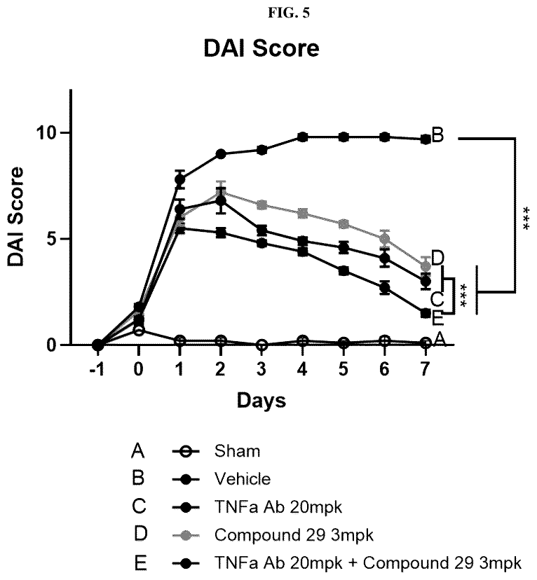

As seen in FIG. 5, the mice group that received TNFα Ab (Line C) showed a therapeutic effect with a decrease of DAI score over the following days. The mice group that received Compound 29 (Line D) showed a decrease in DAI score. The mice group that received the combination of TNFα Ab and Compound 29 (Line E) showed a larger decrease in DAI score over the following days. Sham (Line A) and Vehicle (Line B) were used as controls.

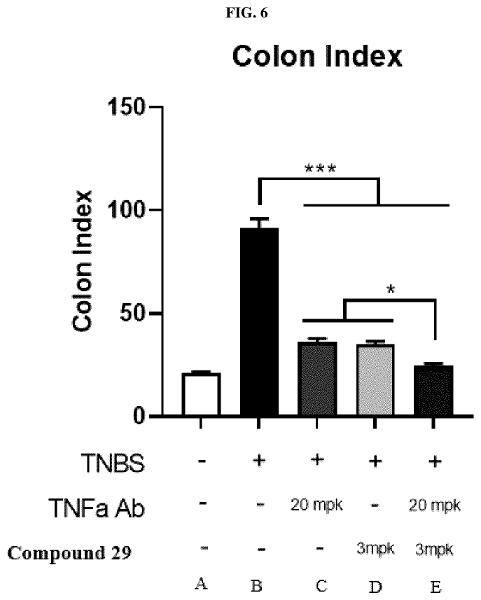

As seen in FIG. 6, the mice group that received TNFα Ab (Bar C) had colon index around 40. The mice group that received Compound 29 (Bar D) had colon index also around 40. The mice group that received the combination of TNFαAb and Compound 29 (Bar E) had colon index around 30. Sham (Bar A) and Vehicle (Bar B) were used as controls.

What is claimed is:

1. A method of treating an inflammatory epithelial disease in a subject in need thereof, the method comprising the administering to the subject:
(a) a compound of Formula (I), or a pharmaceutically acceptable salt, or stereoisomer thereof: 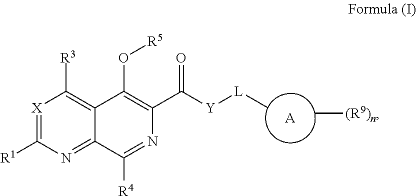
wherein:
R¹ is monocyclic heterocycloalkyl optionally and independently substituted with one or more R^{1a};
each R^{1a} is independently halogen, —CN, —NO₂, —OH, —OR^{a}, —OC(═O)R⁸, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{1a} on the same atom are taken together to form an oxo;
X is N or CR²;
R² is hydrogen, fluoro, chloro, bromo, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R³ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁴ is hydrogen, halogen, —CN, —NO₂, —OH, —OR^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
R⁵ is hydrogen, C₁-C₆alkyl, C₁-C₆haloalkyl, C₁-C₆hydroxyalkyl, C₁-C₆aminoalkyl, or C₁-C₆heteroalkyl;
Y is —O—, —S—, or —NR⁶—;
R⁶ is hydrogen, C₁-C₆alkyl, C₁-C₆haloalkyl, C₁-C₆hydroxyalkyl, C₁-C₆aminoalkyl, or C₁-C₆heteroalkyl;
L is —(CR⁷R⁸)ₚ—;
each R⁷ and R⁸ are independently hydrogen, C₁-C₆alkyl, C₁-C₆haloalkyl, C₁-C₆hydroxyalkyl, C₁-C₆aminoalkyl, or C₁-C₆heteroalkyl;
or R⁷ and R⁸ on the same carbon are taken together to form a cycloalkyl or heterocycloalkyl; each optionally substituted with one or more R^{7a};
each R^{7a} is independently halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl;
p is 0-4;
Ring A is cycloalkyl, heterocycloalkyl, aryl, or heteroaryl;
each R⁹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{9a} on the same atom are taken together to form an oxo;
n is 0-4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; or
two R on the same atom are taken together to form an oxo;
(b) an additional agent or a pharmaceutically acceptable salt thereof;
wherein the combined amount of (a) and (b) is therapeutically effective for the treating of the inflammatory epithelial disease.

2. The method of claim 1, wherein the compound of Formula (I) is a compound of Table 1, or a pharmaceutically acceptable salt, or stereoisomer thereof.

3. The method of claim 1 or 2, wherein the compound of Formula (I) is 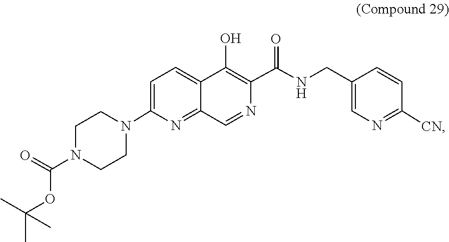 [SMILES: CC(C)(C)OC(=O)N1CCN(c2ccc3c(O)c(C(=O)NCc4ccc(C#N)nc4)ncc3n2)CC1]
or a pharmaceutically acceptable salt thereof, or stereoisomer thereof.

4. The method of any one of claims 1-3, wherein the inflammatory epithelial disease is a disease affecting the respiratory tract, mucosa, skin, gastrointestinal tract, lining of major organs and endocrine glands, vascular tissue, or any combination thereof.

5. The method of any one of claims 1-4, wherein the inflammatory epithelial disease is inflammatory bowel disease.

6. The method of any one of claims 1-4, wherein the inflammatory epithelial disease is ulcerative colitis, Crohn's disease, collagenous colitis, lymphocytic colitis, ischemic colitis, diversion colitis, Behcet's syndrome, or indeterminate colitis.

7. The method of any one of claims 1-6, wherein the additional agent is a 5-aminosalicylates (5-ASAs), anti-inflammatory agent, an ASK1 inhibitor, an alpha-fetoproteinmodulator, an adenosine A3 receptor antagonist, an adrenomedullin ligand, an AKT1 gene inhibitor, an anti-CD28 inhibitor, an antibiotic, an antifungal, an ATPase inhibitor, a beta adrenoceptor antagonist, a BTK inhibitor, a beta-glucuronidase inhibitor, a bradykinin receptor modulator, a calcineurin inhibitor, a chaperonin binding immunoglobulin protein, a calcium channel inhibitor, a cathepsin S inhibitor, a CCR3 chemokine antagonist, a CD40 ligand receptor antagonist, a chemokine CXC ligand inhibitor, a CHST15 gene inhibitor, a collagen modulator, a CSF-1 antagonist, a cyclooxygenase inhibitor, a cytochrome P450 3A4 inhibitor, a carbohydrate metabolism modulator, a CCR9 chemokine antagonist, a CD233 modulator, a CD29 modulator, a CD3 antagonist, a CD4 antagonist, a CD40 ligand inhibitor, a CD40 gene inhibitor, a CX3CR1 chemokine modulator, a COT protein kinase inhibitor, an eotaxin ligand inhibitor, an EP4 prostanoid receptor agonist, an erythropoietin receptor agonist, an ecobiotic, an FIF0 ATP synthase modulator, a farnesoid X receptor (FXR and NR1H4) agonist or modulator, a fecal microbiota transplantation (FMT), a fractalkine ligand inhibitor, a free fatty acid receptor 2 antagonist, a GATA 3 transcription factor inhibitor, a glucagon-like peptide 2 agonist, a glucocorticoid receptor modulator, glucocorticoid receptor agonist, a guanylate cyclase receptor agonist, a histone deacetylase inhibitor, a histone deacetylase-6 inhibitor, an HLA class II antigen modulator, an immunosuppressant, an IL-12 antagonist, an IL-13 antagonist, an IL-23 antagonist, an IL-6 antagonist, an IL-6 receptor modulator, an IL-7 receptor modulator, an IL-7 antagonist, an IL-8 antagonist, an integrin alpha-4/beta-1 antagonist, an integrin alpha-4/beta-7 antagonist, an integrin alpha-E antagonist, an integrin antagonist, an integrin beta-7 antagonist, an interleukin ligand inhibitor, an interleukin-2 ligand, an interleukin receptor 17A antagonist, an interleukin-1 beta ligand, an interleukin-1 beta ligand modulator, an IRAK4 inhibitor, an ICAM1 gene inhibitor, an IL-1 beta ligand modulator, an IL-18 antagonist, an IL-22 agonist, an IL-23A inhibitor, an interleukin 1 like receptor 2 inhibitor, a JAK inhibitor, a JAK tyrosine kinase inhibitor, a Jak1 tyrosine kinase inhibitor, a Jak3 tyrosine kinase inhibitor, an LanC like protein 2 modulator, a lipoxygenase modulator, a lactoferrin stimulator, a leukocyte elastate inhibitor, a leukocyte proteinase-3 inhibitor, a MAdCAM inhibitor, a matrix metalloprotease inhibitor, a melanocortin agonist, a melanocortin MC1 agonist, a metalloprotease-9 inhibitor, a melanin concentrating hormone (MCH-1) antagonist, a microbiome modulator, a natriuretic peptide receptor C agonist, a neuregulin-4 ligand, an NKG2 D activating NK receptor antagonist, an NLRP3 inhibitor, a neuregulin-4 ligand, a nuclear factor kappa B inhibitor, an opioid receptor antagonist, an opioid receptor delta antagonist, an oxidoreductase inhibitor, an OX40 ligand inhibitor, a Pellino homolog 1 inhibitor, a P2X7 purinoceptor agonist, a PDE 4 inhibitor, a phagocytosis stimulating peptide modulator, a potassium channel inhibitor, a PPAR alpha agonist, a PPAR delta agonist, a PPAR gamma agonist, a protein fimH inhibitor, a P-selectin glycoprotein ligand-1 inhibitor, a Ret tyrosine kinase receptor inhibitor, an RNA polymerase inhibitor, a RIP-1 kinase inhibitor, a RIP-2 kinase inhibitor, a tissue transglutaminase inhibitor, a sphingosine-1-phosphate phosphatase-1 stimulator, a sphingosine-1-phosphate phosphatase modulator, a sphingosine-1-phosphate receptor-1 agonist, a sphingosine-1-phosphate receptor-1 antagonist, a sphingosine-1-phosphate receptor-1 modulator, a sphingosine-1-phosphate receptor-5 modulator, a STAT3 gene inhibitor, a stem cell antigen-1 inhibitor, a superoxide dismutase modulator, a superoxide dismutase stimulator, an SYK inhibitor, a TGF beta 1 ligand inhibitor, a thymulin agonist, a TLR antagonist, a TLR agonist, a TNF alpha ligand inhibitor, a TNF antagonist, a tumor necrosis factor superfamily member 14 modulator, a type II TNF receptor modulator, a Tpl 2 inhibitor, a TLR-3 antagonist, a TLR-4 antagonist, a TLR-8 inhibitor, a TLR-9 agonist, a TNF ligand inhibitor, a TNF alpha ligand modulator, a TPL-2 inhibitor, a tumor necrosis factor superfamily member 15 inhibitor, a Tyk2 tyrosine kinase inhibitor, a type I IL-1 receptor antagonist, a TrkA receptor antagonist, a vanilloid VR1 agonist, a zonulin inhibitor, or a pharmaceutically acceptable salt thereof.

8. The method of claim 7, wherein the 5-ASAs is balsalazide, sulphasalazine, mesalazine, olsalazine, mesalamine, or a pharmaceutically acceptable salt thereof.

9. The method of claim 7, wherein the anti-inflammatory agent is a corticosteroid, a non-steroidal anti-inflammatory drug (NSAID), non-specific cyclooxygenase enzyme inhibitor, or COX-2 specific cyclooxygenase enzyme inhibitor, or a pharmaceutically acceptable salt thereof.

10. The method of claim 9, wherein the corticosteroid is cortisone, dexamethasone, methylprednisolone, prednisolone, prednisolone sodium phosphate, prednisone, or a pharmaceutically acceptable salt thereof.

11. The method of claim 9, wherein the NSAID is ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, hydroxychloroquine, or a pharmaceutically acceptable salt thereof.

12. The method of claim 9, wherein the COX-2 specific cyclooxygenase enzyme inhibitor is celecoxib, valdecoxib, lumiracoxib, etoricoxib, rofecoxib, or a pharmaceutically acceptable salt thereof.

13. The method of claim 7, wherein the anti-inflammatory agent is gold sodium thiomalate, auranofin, or a pharmaceutically acceptable salt thereof.

14. The method of claim 7, wherein the immunosuppressant is mercaptopurine, methotrexate, leflunomide, cyclosporine, tacrolimus, azathioprine, mycophenolate mofetil, or a pharmaceutically acceptable salt thereof.

15. The method of claim 14, wherein the immunosuppressant is cyclosporine or a pharmaceutically acceptable salt thereof.

16. The method of claim 7, wherein the ASK1 inhibitor is GS-4997 or a pharmaceutically acceptable salt thereof.

17. The method of claim 7, wherein the beta adrenoceptor antagonist is NM-001 or a pharmaceutically acceptable salt thereof.

18. The method of claim 7, wherein the BTK inhibitor is GS-4059 or a pharmaceutically acceptable salt thereof.

19. The method of claim 7, wherein the calcineurin inhibitor is tacrolimus or ciclosporin or a pharmaceutically acceptable salt thereof.

20. The method of claim 7, wherein the CD40 ligand receptor antagonist is FFP-104, BI-655064, or a pharmaceutically acceptable salt thereof.

21. The method of claim 7, wherein the cathepsin S inhibitor is VBY-129 or a pharmaceutically acceptable salt thereof.

22. The method of claim 7, wherein the chemokine CXC ligand inhibitor is LY-3041658 or a pharmaceutically acceptable salt thereof.

23. The method of claim 7, wherein the CHST15 gene inhibitor is STNM-01 or a pharmaceutically acceptable salt thereof.

24. The method of claim 7, wherein the collagen modulator is ECCS-50 (DCCT-10) or a pharmaceutically acceptable salt thereof.

25. The method of claim 7, wherein the CSF-1 antagonist is JNJ-40346527 (PRV-6527), SNDX-6352, or a pharmaceutically acceptable salt thereof.

26. The method of claim 7, wherein the eotaxin ligand inhibitor is bertilimumab or a pharmaceutically acceptable salt thereof.

27. The method of claim 7, wherein the EP4 prostanoid receptor agonist is KAG-308 or a pharmaceutically acceptable salt thereof.

28. The method of claim 7, wherein the fractalkine ligand inhibitor is quetmolimab (E-6011) or a pharmaceutically acceptable salt thereof.

29. The method of claim 7, wherein the free fatty acid receptor 2 antagonist is GLPG-0974 or a pharmaceutically acceptable salt thereof.

30. The method of claim 7, wherein the GATA 3 transcription factor inhibitor is SB-012 or a pharmaceutically acceptable salt thereof.

31. The method of claim 7, wherein the glucagon-like peptide 2 agonist is teduglutide, apraglutide, or a pharmaceutically acceptable salt thereof.

32. The method of claim 7, wherein the guanylate cyclase receptor agonist is dolcanatide or a pharmaceutically acceptable salt thereof.

33. The method of claim 7, wherein the histone deacetylase inhibitor is givinostat or a pharmaceutically acceptable salt thereof.

34. The method of claim 7, wherein the HLA class II antigen modulator is HLA class II protein modulator.

35. The method of claim 7, wherein the IL-12 antagonist is ustekinumab (IL12/IL23), MEDI2070, or a pharmaceutically acceptable salt thereof.

36. The method of claim 7, wherein the IL-13 antagonist is tralokinumab or a pharmaceutically acceptable salt thereof.

37. The method of claim 7, wherein the IL-23 antagonist is tildrakizumab, risankizumab (BI-655066), mirikizumab (LY-3074828), brazikumab (AMG-139), PTG-200, or a pharmaceutically acceptable salt thereof.

38. The method of claim 7, wherein the IL-6 antagonist is olokizumab or a pharmaceutically acceptable salt thereof.

39. The method of claim 7, wherein the IL-6 receptor modulator is olamkicept or a pharmaceutically acceptable salt thereof.

40. The method of claim 7, wherein the IL-7 receptor antagonist is OSE-127 or a pharmaceutically acceptable salt thereof.

41. The method of claim 7, wherein the IL-8 receptor antagonist is clotrimazole or a pharmaceutically acceptable salt thereof.

42. The method of claim 7, wherein the integrin alpha-4/beta-1 antagonist is natalizumab or a pharmaceutically acceptable salt thereof.

43. The method of claim 7, wherein the integrin alpha-4/beta-7 antagonist is etrolizumab (a4b7/aEb7), vedolizumab, carotegast methyl, TRK-170 (a4b7/a4b1), PN-10943, PTG-100, or a pharmaceutically acceptable salt thereof.

44. The method of claim 7, wherein the integrin antagonist is E-6007 or a pharmaceutically acceptable salt thereof.

45. The method of claim 7, wherein the interleukin ligand inhibitor is bimekizumab (IL-17A/IL-17F) or a pharmaceutically acceptable salt thereof.

46. The method of claim 7, wherein the interleukin receptor 17A antagonist is brodalumab or a pharmaceutically acceptable salt thereof.

47. The method of claim 7, wherein the interleukin-1 beta ligand is K(D)PT or a pharmaceutically acceptable salt thereof.

48. The method of claim 7, wherein the JAK tyrosine kinase inhibitor is tofacitinib, peficitinib, TD-3504, TD-1473, or a pharmaceutically acceptable salt thereof.

49. The method of claim 7, wherein the Jak3 tyrosine kinase inhibitor is PF-06651600 or a pharmaceutically acceptable salt thereof.

50. The method of claim 7, wherein the LanC like protein 2 modulator is BT-11 or a pharmaceutically acceptable salt thereof.

51. The method of claim 7, wherein the MAdCAM inhibitor is SHP-647 (PF-547659) or a pharmaceutically acceptable salt thereof.

52. The method of claim 7, wherein the melanocortin MC1 receptor agonist is ASP-3291, PL-8177, or a pharmaceutically acceptable salt thereof.

53. The method of claim 7, wherein the natriuretic peptide receptor C agonist is plecanatide or a pharmaceutically acceptable salt thereof.

54. The method of claim 7, wherein the NKG2 D activating NK receptor antagonist is JNJ-4500 or a pharmaceutically acceptable salt thereof.

55. The method of claim 7, wherein the opioid receptor antagonist is naltrexone, IRT-103, or a pharmaceutically acceptable salt thereof.

56. The method of claim 7, wherein the oxidoreductase inhibitor is olsalazine or a pharmaceutically acceptable salt thereof.

57. The method of claim 7, wherein the P2X7 purinoceptor modulator is SGM-1019 or a pharmaceutically acceptable salt thereof.

58. The method of claim 7, wherein the PDE 4 inhibitor is apremilast or a pharmaceutically acceptable salt thereof.

59. The method of claim 7, wherein the PPAR alpha agonist or PPAR delta agonist is elafibranor (GFT-1007) or a pharmaceutically acceptable salt thereof.

60. The method of claim 7, wherein the PPAR gamma agonist is GED-0507-34-Levo or a pharmaceutically acceptable salt thereof.

61. The method of claim 7, wherein the protein fimH inhibitor is sibofimloc (EB-8018) or a pharmaceutically acceptable salt thereof.

62. The method of claim 7, wherein the P-selectin glycoprotein ligand-1 inhibitor is SEL-K2, AbGn-168H, neihulizumab, or a pharmaceutically acceptable salt thereof.

63. The method of claim 7, wherein the sphingosine-1-phosphate phosphatase 1 stimulator is etrasimod or a pharmaceutically acceptable salt thereof.

64. The method of claim 7, wherein the sphingosine-1-phosphate receptor-1 agonist is ozanimod, mocravimod (KRP-203), BMS-986166, or a pharmaceutically acceptable salt thereof.

65. The method of claim 7, wherein the sphingosine-1-phosphate receptor-5 agonist is ozanimod or a pharmaceutically acceptable salt thereof.

66. The method of claim 7, wherein the sphingosine-1-phosphate receptor-1 antagonist is amiselimod (MT-1303) or a pharmaceutically acceptable salt thereof.

67. The method of claim 7, wherein the sphingosine-1-phosphate receptor-1 modulator is OPL-002 or a pharmaceutically acceptable salt thereof.

68. The method of claim 7, wherein the stem cell antigen-1 inhibitor is ampion (DMI-9523) or a pharmaceutically acceptable salt thereof.

69. The method of claim 7, wherein the superoxide dismutase modulator is midismase or a pharmaceutically acceptable salt thereof.

70. The method of claim 7, wherein the SYK inhibitor is GS-9876 or a pharmaceutically acceptable salt thereof.

71. The method of claim 7, wherein the TNF alpha ligand inhibitor is adalimumab, certolizumab pegol, infliximab, golimumab, DLX-105, Debio-0512, HMPL-004, CYT-020-TNFQb, Hemay-007, V-565, anti-TNFα antibody, or a pharmaceutically acceptable salt thereof.

72. The method of claim 71, wherein the TNF alpha ligand inhibitor is anti-TNFα antibody or a pharmaceutically acceptable salt thereof.

73. The method of claim 7, wherein the tumor necrosis factor superfamily member 14 modulator is AEVI-002 or a pharmaceutically acceptable salt thereof.

74. The method of claim 7, wherein the TNF antagonist is AVX-470, tulinercept, etanercept, or a pharmaceutically acceptable salt thereof.

75. The method of claim 7, wherein the zonulin inhibitor is larazotide acetate or a pharmaceutically acceptable salt thereof.

76. The method of claim 7, wherein the CD40 ligand inhibitor is SAR-441344, letolizumab, or a pharmaceutically acceptable salt thereof.

77. The method of claim 7, wherein the adenosine A3 receptor antagonist is PBF-677 or a pharmaceutically acceptable salt thereof.

78. The method of claim 7, wherein the adrenomedullin ligand is adrenomedullin or a pharmaceutically acceptable salt thereof.

79. The method of claim 7, wherein the antibiotic is ciprofloxacin, clarithromycin, metronidazole, vancomycin, rifamycin, rifaximin, tosufloxacin, or a pharmaceutically acceptable salt thereof.

80. The method of claim 7, wherein the alpha-fetoprotein modulator is ACT-101 or a pharmaceutically acceptable salt thereof.

81. The method of claim 7, wherein the carbohydrate metabolism modulator is ASD-003 or a pharmaceutically acceptable salt thereof.

82. The method of claim 7, wherein the CCR9 chemokine antagonist is CCX-507 or a pharmaceutically acceptable salt thereof.

83. The method of claim 7, wherein the CD233 modulator is GSK-2831781 or a pharmaceutically acceptable salt thereof.

84. The method of claim 7, wherein the CD29 modulator is PF-06687234 or a pharmaceutically acceptable salt thereof.

85. The method of claim 7, wherein the CD3 antagonist is NI-0401 or a pharmaceutically acceptable salt thereof.

86. The method of claim 7, wherein the CX3CR1 chemokine modulator is E-6130 or a pharmaceutically acceptable salt thereof.

87. The method of claim 7, wherein the ecobiotic is SER-287 or a pharmaceutically acceptable salt thereof.

88. The method of claim 7, wherein the F1F0 ATP synthase modulator is LYC-30937 EC or a pharmaceutically acceptable salt thereof.

89. The method of claim 7, wherein the glucocorticoid receptor modulator is ABBV-3373 or a pharmaceutically acceptable salt thereof.

90. The method of claim 7, wherein the TNF ligand inhibitor is ABBV-3373 or a pharmaceutically acceptable salt thereof.

91. The method of claim 7, wherein the ICAM1 gene inhibitor is alicaforsen or a pharmaceutically acceptable salt thereof.

92. The method of claim 7, wherein the IL-18 antagonist is GSK-1070806 or a pharmaceutically acceptable salt thereof.

93. The method of claim 7, wherein the IL-22 agonist is RG-7880 or a pharmaceutically acceptable salt thereof.

94. The method of claim 7, wherein the IL-23A inhibitor is guselkumab or a pharmaceutically acceptable salt thereof.

95. The method of claim 7, wherein the interleukin 1 like receptor 2 inhibitor is BI-655130 or a pharmaceutically acceptable salt thereof.

96. The method of claim 7, wherein the lactoferrin stimulator is recombinant human lactoferrin (VEN-100) or a pharmaceutically acceptable salt thereof.

97. The method of claim 7, wherein the leukocyte elastase inhibitor is tiprelestat or a pharmaceutically acceptable salt thereof.

98. The method of claim 7, wherein the leukocyte proteinase-3 inhibitor is tiprelestat or a pharmaceutically acceptable salt thereof.

99. The method of claim 7, wherein the melanin concentrating hormone (MCH-1) antagonist is CSTI-100 or a pharmaceutically acceptable salt thereof.

100. The method of claim 7, wherein the metalloprotease-9 inhibitor is GS-5745 or a pharmaceutically acceptable salt thereof.

101. The method of claim 7, wherein the NLRP3 inhibitor is dapansutrile, BMS-986299, SB-414, MCC-950, IFM-514, JT-194, PELA-167, NBC-6, or a pharmaceutically acceptable salt thereof.

102. The method of claim 7, wherein the farnesoid X receptor (FXR and NR1H4) agonist or modulator is AGN-242266, cilofexortromethamine (GS-9674), EDP-305, EYP-001, GNF-5120, MET-409, nidufexor (LMB-763), obeticholic acid, TERN-101, tropifexor, or a pharmaceutically acceptable salt thereof.

103. The method of claim 7, wherein the nuclear factor kappa B inhibitor is thetanix or a pharmaceutically acceptable salt thereof.

104. The method of claim 7, wherein the OX40 ligand inhibitor is KHK-4083 or a pharmaceutically acceptable salt thereof.

105. The method of claim 7, wherein the Pellino homolog 1 inhibitor is BBT-401 or a pharmaceutically acceptable salt thereof.

106. The method of claim 7, wherein the Ret tyrosine kinase receptor inhibitor is GSK-3179106 or a pharmaceutically acceptable salt thereof.

107. The method of claim 7, wherein the RIP-1 kinase inhibitor is GSK-2982772 or a pharmaceutically acceptable salt thereof.

108. The method of claim 7, wherein the RIP-2 kinase inhibitor is GSK-2983559 or a pharmaceutically acceptable salt thereof.

109. The method of claim 7, wherein the tissue transglutaminase inhibitor is zampilimab or a pharmaceutically acceptable salt thereof.

110. The method of claim 7, wherein the TLR-3 antagonist is PRV-300 or a pharmaceutically acceptable salt thereof.

111. The method of claim 7, wherein the TLR-4 antagonist is JKB-122 or a pharmaceutically acceptable salt thereof.

112. The method of claim 7, wherein the TLR-8 inhibitor is E-6887, IMO-4200, IMO-8400, IMO-9200, MCT-465, MEDI-9197, motolimod, resiquimod, VTX-1463, VTX-763, or a pharmaceutically acceptable salt thereof.

113. The method of claim 7, wherein the TLR-9 agonist is cobitolimod, IMO-2055, IMO-2125, lefitolimod, litenimod, MGN-1601, PUL-042, or a pharmaceutically acceptable salt thereof.

114. The method of claim 7, wherein the TPL-2 inhibitor is GS-4875 or a pharmaceutically acceptable salt thereof.

115. The method of claim 7, wherein the tumor necrosis factor superfamily member 15 inhibitor is PF-06480605, PRA023, or a pharmaceutically acceptable salt thereof.

116. The method of claim 7, wherein the Tyk2 tyrosine kinase inhibitor is PF-06826647, BMS-986165, or a pharmaceutically acceptable salt thereof.

117. The method of claim 7, wherein the Type I IL-1 receptor antagonist is anakinra or a pharmaceutically acceptable salt thereof.

118. The method of claim 7, wherein the anti-CD28 inhibitor is JNJ-3133, abatacept, or a pharmaceutically acceptable salt thereof.

119. The method of claim 7, wherein the CD4 antagonist is IT-1208 or a pharmaceutically acceptable salt thereof.

120. The method of claim 7, wherein the CD40 gene inhibitor is NJA-730 or a pharmaceutically acceptable salt thereof.

121. The method of claim 7, wherein the chaperonin binding immunoglobulin protein is IRL-201805 or a pharmaceutically acceptable salt thereof.

122. The method of claim 7, wherein the COT protein kinase inhibitor is GS-4875 or a pharmaceutically acceptable salt thereof.

123. The method of claim 7, wherein the glucocorticoid receptor agonist is budesonide, beclomethasone dipropionate, dexamethasone sodium phosphate, or a pharmaceutically acceptable salt thereof.

124. The method of claim 7, wherein the histone deacetylase-6 inhibitor is CKD-506 or a pharmaceutically acceptable salt thereof.

125. The method of claim 7, wherein the neuregulin-4ligand is NRG-4 or a pharmaceutically acceptable salt thereof.

126. The method of claim 7, wherein the microbiome modulator is ABI-M201 or a pharmaceutically acceptable salt thereof.

127. The method of claim 7, wherein the TrkA receptor antagonist is SNA-125 or a pharmaceutically acceptable salt thereof.

128. The method of claim 7, wherein the JAK inhibitor is AT9283, AZD1480, baricitinib, BMS-911543, fedratinib, filgotinib (GLPG0634), gandotinib (LY2784544), INCB039110, lestaurtinib, momelotinib (CYT0387), NS-018, pacritinib (SB1518), peficitinib (ASP015K), ruxolitinib, tofacitinib, XL019, upadacitinib (ABT-494), filgotinib, GLPG-0555, SHR-0302, brepocitinib (PF-06700841), or a pharmaceutically acceptable salt thereof.

129. The method of any one of claims 1-5, wherein the additional agent comprises obefazimod (ABX-4640), adalimumab, alicaforsen, ALLO-ASC-CD, AMG-966, anakinra, apremilast, Alequel, AMG-139, amiselimod, ASD-003, ASP-3291, AX-1505, BBT-401, balsalazide, beclomethasone dipropionate, BI-655130, BMS-986184, budesonide, CEQ-508, certolizumab, ChAdOx2-HAV, dexamethasone sodium phosphate, DNVX-078, etanercept, cibinetide, Clostridium butyricum, ETX-201, golimumab, GS-4997, GS-9876, GS-4875, GS-4059, infliximab, mesalazine, HLD-400, LYC-30937 EC, IONIS-JB11-2.5Rx, JNJ-64304500, JNJ-4447, naltrexone, natalizumab, neihulizumab, olsalazine, PH-46-A, propionyl-L-camitine, PTG-100, remestemcel-L, tacrolimus, teduglutide, tofacitinib, ASP-1002, ustekinumab, vedolizumab, AVX-470, INN-108, SGM-1019, PF-06480605, PF-06651600, PF-06687234, RBX-8225, SER-287, Thetanix, TOP-1288, VBY-129, 99mTc-annexin V-128, bertilimumab, DLX-105, dolcanatide, FFP-104, filgotinib, foralumab, GED-0507-34-Levo, givinostat, GLPG-0974, iberogast, JNJ-40346527, K(D)PT, KAG-308, KHK-4083, KRP-203, larazotide acetate, LY-3074828, midismase, olokizumab, OvaSave, P-28-GST, PF-547659, prednisolone, QBECO, RBX-2660, RG-7835, JKB-122, SB-012, STNM-01, Debio-0512, TRK-170, zucapsaicin, ABT-494, Ampion, BI-655066, carotegast methyl, cobitolimod, elafibranor, etrolizumab, GS-5745, HMPL-004, LP-02, ozanimod, peficitinib, quetmolimab (E-6011), RHB-104, rifaximin, tildrakizumab, tralokinumab, brodalumab, laquinimod, plecanatide, vidofludimus, AZD-058, or a pharmaceutically acceptable salt thereof.
